(19) United States
(12) Patent Application Publication
(10) Pub. No.: US 2024/0050389 A1
(43) Pub. Date: Feb. 15, 2024

(54) TREATMENT OF IDIOPATHIC PULMONARY FIBROSIS

(71) Applicant: Imperial College Innovations Limited, London (GB)

(72) Inventors: Adam Byrne, IV, London (GB); Toby Maher, Hertfordshire (GB); Clare Lloyd, London (GB); Pia Patricia Ogger, London (GB)

(21) Appl. No.: 18/245,157

(22) PCT Filed: Sep. 15, 2020
(86) PCT No.: PCT/GB20/52218

§ 371 Date: Mar. 14, 2023

(51) Int. Cl. A61K 31/194; A61K 35/15; A61P 11/00; G01N 33/50

(52) U.S. Cl. CPC A61K 31/194; A61K 35/15; A61P 11/00; G01N 33/5044

(57) ABSTRACT

The invention generally relates to products for use in the treatment and/or prevention of tissue fibrosis, and to methods of identifying agents which inhibit tissue fibrosis. More specifically, the invention relates to agents that inhibit succinate dehydrogenase, and monocyte-recruited macrophage populations, for use in the treatment and/or prevention of tissue fibrosis.

### FIELD OF THE INVENTION

The invention generally relates to products for use in the treatment and/or prevention of tissue fibrosis, and to methods of identifying agents which inhibit tissue fibrosis. More specifically, the invention relates to agents that inhibit succinate dehydrogenase, and monocyte-recruited macrophage populations, for use in the treatment and/or prevention of tissue fibrosis.

### BACKGROUND OF THE INVENTION

Idiopathic pulmonary fibrosis (IPF) is a chronic debilitating lung disease, characterized by the deposition of excessive extracellular matrix in the lung parenchyma. Existing pharmacological options are limited and with an increasing worldwide incidence and a median survival of 3 years from diagnosis, there is an urgent requirement to understand pathological mechanisms involved and to provide effective treatments.

A growing body of evidence supports a role for airway macrophages (AMs) in regulating pathogenic mechanisms underlying IPF. AMs are crucial in contributing to pulmonary defence, repair, surfactant processing and inflammatory responses. Moreover, AMs are strategically positioned at the interface between the airways and the environment and are found in the alveoli and airways, secreting numerous pro-fibrotic soluble mediators, chemokines, and matrix metalloproteases. Macrophages demonstrate remarkable plasticity and are capable of acquiring phenotypes which can both drive or resolve fibro-proliferative responses to injury. For example, AMs have been shown to be involved in the regulation of the extracellular matrix via secretion of matrix metalloproteases (MMPs) or by direct uptake of collagen.

Macrophage activation is tightly linked to cellular metabolism. Inflammatory activation of macrophages results in impaired mitochondrial respiration and tricarboxylic acid (TCA) cycle disruption, resulting in the accumulation of endogenous metabolites capable of adopting immunomodulatory roles. One such bioactive metabolite is itaconate. In macrophages, synthesis of itaconate is catalyzed by cis-aconitate decarboxylase (CAD), encoded by aconitate decarboxylase 1 (ACOD1), which mediates the decarboxylation of cis-aconitate to itaconate. Itaconate is one of the most highly induced metabolites in activated bone marrow-derived macrophages and can suppress the expression of pro-inflammatory cytokines. Furthermore, itaconate has been shown to control macrophage effector functions via competitive inhibition of succinate dehydrogenase (SDH) mediated oxidation of succinate and furthermore, drives an anti-inflammatory program via the KEAP-1-NRF2 axis. Therefore, itaconate appears to be a crucial regulator of macrophage phenotype and function. However, its functional significance in specialized tissue resident macrophages during chronic respiratory disease such as IPF remains unknown.

### SUMMARY OF THE INVENTION

The inventors have previously linked AM phenotype to disease outcome in IPF, since increased numbers of AMs lacking the transferrin receptor CD71 are associated with worsened disease. The inventors have now demonstrated that the ACOD1/itaconate axis is an endogenous pulmonary regulatory pathway, which limits fibrosis. Further, the inventors have sown for the first time that itaconate and cis-aconitate decarboxylase have therapeutic potential as targets in IPF and other diseases where fibrosis plays a role, particularly chronic respiratory diseases with a fibrotic component. In particular, the present inventors have now demonstrated for the first time that the ACOD1/itaconate axis is altered in the human lung during IPF, itaconate is an anti-fibrotic factor in the murine lung and that it impairs human fibroblast activity. The inventors have also shown that in patients with IPF, there is decreased expression of ACOD1 in AMs and reduced levels of airway itaconate, compared to healthy controls. Acod1 deficiency in mice leads to more severe disease pathology, which is further exacerbated by adoptive transfer of Acod1−/−, but not WT monocyte-recruited AMs. Addition of exogenous itaconate to cultures of human lung fibroblasts limits proliferation and wound healing and furthermore, inhaled itaconate (particularly oropharyngeal inhalation) ameliorates lung fibrosis in mice.

Accordingly, the invention provides an agent which inhibits succinate dehydrogenase for use in the treatment or prevention of tissue fibrosis.

The agent which inhibits succinate dehydrogenase may directly inhibit succinate dehydrogenase, wherein optionally the agent is selected from a small molecule compound, a nucleic acid, an antibody or antigen-binding fragment thereof, or an aptamer.

The agent which inhibits succinate dehydrogenase may indirectly inhibit succinate dehydrogenase, wherein optionally the agent increases the expression and/or activity of aconitate decarboxylase 1 (ACOD1), wherein optionally the agent is a nucleic acid, a protein, or a small molecule,.

The small molecule compound which directly inhibits succinate dehydrogenase may be itaconate or a derivative or analogue thereof, optionally in the form of a pharmaceutically acceptable salt.

The agent for use in the treatment or prevention of tissue fibrosis may be administered by: inhalation; intraperitoneal, subcutaneous, and/or intramuscular injection; infusion; and/or orally, preferably wherein the agent is administered by oropharyngeal inhalation and/or nasal inhalation.

The agent for use in the treatment or prevention of tissue fibrosis may be delivered in a drug delivery system, wherein optionally said drug delivery system: a) specifically targets phagocytes; and/or b) is a liposome-based drug delivery system.

The itaconate, derivative, analogue or pharmaceutically acceptable salt thereof may be administered at a dose of about 0.1 mg/kg to about 10 mg/kg.

The itaconate, derivative, analogue or pharmaceutically acceptable salt thereof is administered once per week to about four times per day, preferably about once per day.

The invention also provides a population of monocyte-recruited macrophages (Mo-Ms) for use in the treatment or prevention of tissue fibrosis.

The Mo-Ms may express Acod1, and optionally have a quiescent metabolic phenotype.

The population of Mo-Ms may be administered directly to an individual to be treated. The population of Mo-Ms may be recruited following administration of a composition which stimulates targeting of Mo-Ms to a tissue to be treated.

The Mo-Ms may be autologous Mo-Ms or allogenic Mo-Ms.

According to the present invention, the treatment or prevention may modify the metabolic and/or fibrotic phenotype of tissue-resident macrophages (Tr-Ms), preferably wherein the treatment or prevention increases the metabolic phenotype and/or reduces the fibrotic phenotype of the Tr-M.

The treatment or prevention may increase the proportion of CD11b⁺/MHCII⁺ Tr-Ms resident in the tissue.

The treatment or prevention may modify the metabolic and/or fibrotic phenotype of fibroblasts within the tissue, preferably wherein the treatment or prevention reduces the metabolic and/or fibrotic phenotype of the fibroblasts.

The treatment or prevention may: reduce the oxygen consumption rate, maximal respiration and/or spare respiratory capacity of fibroblasts; reduce proliferation of fibroblasts; and/or reduce the wound healing capacity of fibroblasts.

The treatment or prevention may result in: an improvement in the fibrosis of the tissue; a decrease in tissue collagen expression, preferably Col3α1, Col1α1 and/or Col4α1; a decrease in tissue fibronectin (Fn1) expression; a decrease in IL-1β expression in fibroblasts obtained from the tissue; and/or a decrease in hydroxyproline levels.

According to the invention, the fibrosis may be pulmonary fibrosis, liver fibrosis, kidney fibrosis, intestinal fibrosis, cardiac fibrosis, myelofibrosis and/or skin fibrosis.

The pulmonary fibrosis is any form of chronic fibrosing interstitial lung disease including idiopathic pulmonary fibrosis.

The treatment or prevention according to the invention may result in: a) an improvement in lung function, preferably an increase in forced vital capacity, an increase in total lung capacity and/or an increase in the transfer capacity of the lung for the uptake of carbon monoxide, as measured by gas transfer (TLco) test; b) a reduction in the decline of forced vital capacity; c) preservation or improvement of exercise capacity; d) a reduction in the progression of fibrosis as quantified by high resolution computed tomography; e) preservation or improvement of quality of life; and/or (f) improved survival.

The agent or population of Mo-Ms for use according to the invention may be for use in an individual having reduced levels of itaconate in a sample of the tissue to be treated, and wherein the tissue fibrosis is pulmonary fibrosis, the sample is optionally a bronchoalveolar lavage (BAL) sample.

The agent or population of Mo-Ms may be for use in an individual having Tr-Ms with reduced ACOD1 expression.

The agent or population of Mo-Ms may be for use in combination with another therapeutic.

The invention also provides a method for identifying a compound which inhibits fibrosis progression, comprising the steps of: a) culturing cells in vitro; b) adding a test compound to the cultured cells; and c) determining a change in the metabolic phenotype of the cells in response to the test compound; wherein the change in metabolic phenotype of the cells is a reduction or increase in the metabolic phenotype of the cells; wherein preferably the cells are fibroblasts or Tr-Ms.

In accordance with a method of the invention, a reduction or increase in the metabolic phenotype of the cells may be: a) a reduction or increase in the oxygen consumption rate of the cells; b) a reduction or increase in the maximal respiration of the cells; and/or c) a reduction or increase in the spare respiratory capacity of the cells.

The method of the invention may further comprise a step of determining a reduction in the fibrotic phenotype of the cells in response to the test compound.

### BRIEF DESCRIPTION OF THE DRAWINGS

FIG. 1: The ACOD1/itaconate axis is decreased in IPF and Acod1−/− mice have worsened phenotype upon bleomycin exposure. (A) Gene expression analysis of ACOD1 in CD206+ sorted AMs from human control (n=10) and IPF (n=27) donors. Actb was used as housekeeping gene. (B) Targeted GC-MS analysis of itaconate in bronchoalveolar lavage (BAL) of human control (n=10) and IPF (n=47) donors, normalised to total protein (ng/μg protein). (C) Schematic of dosing regimen. WT or Acod1−/− mice were dosed oropharyngeal with 0.05U bleomycin or PBS control at day 0 and harvested at day 7, day 21 or day 42 post bleomycin. (D) Gene expression analysis of Acod1 in lung homogenates of PBS or Bleo dosed mice at day 7, day 21 and day 42 post bleomycin administration; n=3-8 per group, pooled from two independent experiments. (E) Targeted GC-MS analysis of itaconate in BAL of PBS or Bleo dosed mice at day 7, day 21 and day 42 post bleomycin administration; n=3-8 per group, pooled from two independent experiments. (F-H) Total BAL cells (F), numbers of BAL AMs (G) and BAL neutrophils (H) in PBS or Bleo dosed WT and Acod1−/− mice at day 42; n=3-8 per group, pooled from two independent experiments. (I) Resistance, elastance and compliance at baseline measured by FlexiVent in PBS or Bleo dosed WT and Acod1−/− mice at day 42 (PBS n=4-7, Bleo n=10 -12), pooled from two independent experiments and representative of n=3 individual experiments. Data presented as mean±S.D. Statistical significance tested by Mann-Whitney U test or One-Way ANOVA+Sidak's multiple comparison test , *P<0.05, **P<0.01, ***P<0.005, ****P<0.001.

FIG. 2: Acod1−/− mice have worsened fibrotic phenotype at late time point. (A) Gene expression analysis of Col1α1 Col3α1, Col4α1 and Fn1 in lung homogenate of PBS or Bleo dosed WT and Acod1−/− mice at day 21 (n=3-8 per group). Actb was used as housekeeping gene. Pooled from two independent experiments. (B) Fold change hydroxyproline increase in bleomycin compared to PBS in WT and Acod1−/− mice at day 42 post bleomycin (n=4-5 per group), representative of three experiments. (C-D) Ashcroft score (C) and representative images (D) of lung slices of PBS or Bleo dosed mice at day 42 post bleomycin stained with Sirius Red, scored blinded by 3-5 individuals. (E) MFI of MitoSOX red superoxide stain in lungs of PBS or Bleo WT and Acod1−/− mice at day 42 post Bleo (n=7-12 per group), pooled from two independent experiments and representative of n=3 individual experiments. Data presented as mean±S.D. Statistical significance tested by Mann-Whitney U test or One-Way ANOVA+Sidak's multiple comparison test, *P<0.05, **P<0.01.

FIG. 3: Itaconate controls tissue resident AM metabolism. (A) Analysis of the oxygen consumption rate (OCR) of PBS tissue resident-AM (Tr-AM) (n=3), Bleo Tr-AM (n=4) and Bleo monocyte recruited-AM (Mo-AM, n=5) during mitochondrial stress test, assessed after injection of Oligomycin, FCCP and Rotenone/Antimycin A; representative of three independent experiments. (B) Energy map of indicating overall energy state of PBS Tr-AM, Bleo Tr-AM and Bleo Mo-AM; four energy states are shown: quiescent, energetic, aerobic and glycolytic. Same n numbers as in A. (C) Gene expression analysis of Acod1 in BAL PBS Tr-AM, Bleo Tr-AM and Bleo Mo-AM at day 1, day 7 and day 21 (n=4-7 per group) post bleomycin. Actb was used as housekeeping control. Pooled from three independent experiments. (D) Analysis of the OCR of Bleo WT Mo-AM (n=5) and Acod1−/− Mo-AM (n=5) during mitochondrial stress test, assessed as in A; data from two experiments pooled. (E) Maximal respiration during mitochondrial stress test (D), defined as the maximal oxygen consumption rate after addition of FCCP. (F) Spare respiratory capacity (SRC) during mitochondrial stress test (D), defined as subtraction of basal from maximal OCR. (G) Basal extracellular acidification rate (SCAR) as surrogate for glycolysis during mitochondrial stress test (D). (H) Analysis of the OCR of Bleo WT Tr-AM (n=4) and Acod1−/− Tr-AM (n=4) during mitochondrial stress test, assessed as in A; data from two experiments pooled. (I) Maximal respiration during mitochondrial stress test (G), calculated as in (E). (J) SRC during mitochondrial stress test (G), defined as in (F). (K) Basal extracellular acidification rate (SCAR) as surrogate for glycolysis during mitochondrial stress test (H). Tr-AM and Mo-AM were sorted at day 7 post bleomycin. Data presented as mean±S.D. Significance tested by One-Way ANOVA+Sidak's multiple comparison test, *P<0.05, **P<0.01, ***P<0.001. Each data point represents 2-3 mice pooled.

FIG. 4: Acod1-deficient tissue resident AMs are more pro-fibrotic post bleomycin. (A-B) Volcano plots showing differentially expressed genes in WT vs Acod1−/− Tr-AM (A) and Mo-AM (B), 7 days post bleomycin exposure (n=3-4 per group). Genes significantly (p<0.05) up-regulated in WT vs Acod1−/− highlighted in red, while genes significantly downregulated are shown in blue. (C—D) Heat map representation of murine fibrosis gene array of FACS sorted Mo-AM (C) and Tr-AM (D) from WT and Acod1−/− mice. Data shown as log10 of ΔΔCT WT vs Acod1−/−. (E) Representative images of FACS sorted Tr-AM and Mo-AM WT and Acod1−/− mice after cytospin and Diff-Quick staining. Significance tested by Two-tailed T-test *P<0.05.

FIG. 5: Adoptive transfer of WT Mo-AMs improves pulmonary fibrosis and rescues tissue resident AM phenotype in Acod1−/− mice post bleomycin. (A) Schematic of dosing regimen and adoptive transfer. WT or Acod1−/− mice were dosed oropharyngeal with 0.05U bleomycin at day 0, Mo-AMs were FACS sorted at day 7 post bleomycin and transferred into Acod1−/− mice via the oropharyngeal route. Mice were then harvested after further 14 days, at day 21 post initial bleomycin exposure. (B-C) Ashcroft score (D) and representative images (E) of lung slices of Acod1−/− mice adoptively transferred with WT or Acod1−/− Mo-AMs; day 21 post bleomycin stained with Sirius Red, scored blinded by 3-5 individuals. (D) Gene expression analysis of Co1α1, Col3α1, Col4α1 and Fn1 in lung homogenate of Acod1−/− mice adoptively transferred with WT or Acod1−/− Mo-AMs (n=3-6 per group); day 21 post bleomycin, Actb was used as housekeeping gene. (E-H) Fraction of CD11b+/MHC II+ and CD11b-/MHC II- Tr-AM (E-F) and Mo-AM (G-H) in BAL of Acod1−/− mice adoptively transferred with WT or Acod1−/− Mo-AMs; day 21 post bleomycin. Data presented as mean±S.D. Significance tested by Mann Whitney U test, *P<0.05.

FIG. 6: Exogenous itaconate limits human lung fibroblast wound healing. (A) Analysis of the OCR of healthy or IPF primary human lung fibroblasts stimulated for 24 h with either RPMI medium (con) or itaconate (IA) during mitochondrial stress test, assessed after injection of Oligomycin, FCCP and Rotenone/Antimycin A (all groups n=3). (B) Energy map of (A) showing four energy states during mitochondrial stress test: quiescent, energetic, aerobic and glycolytic. Same n numbers as in A. (C) Maximal respiration and spare respiratory capacity (SRC) during mitochondrial stress test (A). Maximal respiration defined as the maximal oxygen consumption rate after addition of FCCP; SRC defined as subtraction of basal from maximal OCR. (D) Proliferation rate of healthy (n=3) human primary lung fibroblasts stimulated with 10 mM itaconate or vehicle control measured using the JULI Stage system. (E) Wound healing capacity of healthy (n=3) human primary lung fibroblasts stimulated with 10 mM itaconate or vehicle control measured using the JuLI Stage system. Two-tailed, unpaired t-test of area under the curve. (F) Gene expression analysis of FN1 and IL-1β in healthy human primary lung fibroblasts stimulated for 24 h with 10 mM itaconate or vehicle control. IA=itaconate. Data presented as mean±S.D. Significance was tested by One Way ANOVA+Sidak's multiple comparison test (A-C), Mann Whitney U test of arear under the curve (D-E) or one-sample t-test against value of 1.0 (F). *P<0.05, **P<0.01.

FIG. 7: Inhaled itaconate is anti-fibrotic. (A) Schematic of dosing regime using 8-10 week old C57BI/6 mice. 0.05U Bleomycin or PBS control and 0.25 mg/kg itaconate or PBS control was administered oropharyngeal at indicated time points and mice were harvested at day 21 post bleomycin. (B-C) Ashcroft score and representative images (C) of lung slices stained with Sirius Red, scored blinded by 3-5 individuals. (D) Gene expression analysis of Col1α1, Col3α1, Col4α1 and Fn1 in lung homogenate; Actb was used as housekeeping control. Pooled from two independent experiments. (E) Resistance, elastance and compliance at baseline measured by FlexiVent in PBS and bleomycin dosed mice treated with 0.25 mg/kg itaconate or vehicle control. Pooled from two independent experiments. Data presented as mean±S.D.; n=7-11 per group. Significance was tested by One-Way ANOVA+Sidak's multiple comparison test *P<0.05.

FIG. 8: MACS sorting of human CD206+ BAL AM. (A) Representative FACS plots showing gating strategy to assess purity of human CD45+, CD206+ BAL AM population before and after magnetic activated cell sorting (MACS). (B) Purity of human CD45+, CD206+ BAL AM population confirmed by FACS, before and after MACS (n=29). (C) Linear regression analysis between relative gene expression of ACOD1 vs ACTB and age in healthy controls (n=10) and IPF patients (n=27). (D) Linear regression analysis between itaconate measured in BAL supernatant by targeted GC-MS, normalised to total protein (ng/μg protein) and age in healthy controls (n=10) and IPF patients (n=47). Data presented as mean±S.D.

FIG. 9: Gating strategies of murine flow cytometry. (A) Representative FACS plots showing gating strategy to determine the following immune cell populations in murine BAL and lung: eosinophils, monocytes, airway macrophages (AM), including monocyte-recruited AMs (SigFint), and tissue-resident AMs (SigFhigh), neutrophils, dendritic cells (DCs), T cells, including Th1-T-cells, Th2-T-cells and Th-17-T-cells. (B) Representative FACS plots showing gating strategy to determine expression of mitoSOX dye for superoxide in murine BAL CD45+ population.

FIG. 10: Lung function and immune cell profile after bleomycin treatment in Acod1−/− mice. (A) Resistance, elastance and compliance at baseline measured by FlexiVent in PBS and bleomycin dosed WT and Acod1−/− mice at day 7 post bleomycin (all groups n=6). (B) Total BAL cells in PBS and bleomycin dosed WT and Acod1−/− mice at day 7 post bleomycin (all groups n=6). (C) Resistance, elastance and compliance at baseline measured by FlexiVent in PBS and bleomycin dosed WT and Acod1−/− mice at day 21 post bleomycin; (n=5-10, two experiments pooled). (D) Total BAL cells in PBS and bleomycin dosed WT and Acod1−/− mice at day 21 post bleomycin; (n=5 — 10, two experiments pooled). (E) Total BAL AMs cells in PBS and bleomycin dosed WT and Acod1−/− mice at day 7 (n=6 all groups) and day 21 (n=5 -10) post bleomycin. (F) Total BAL neutrophils in PBS and bleomycin dosed WT and Acod1−/− mice at day 7 (n=6 all groups) and day 21 (n=5-10) post bleomycin. (G-H) Total T-cells and NK-cells in BAL of PBS and bleomycin dosed WT and Acod1−/− mice at day 7, day 21 and day 42 (n=3-6 per group) post bleomycin. Representative of two experiments. Data presented as mean±S.D.; Significance was tested by One Way ANOVA +Sidak's multiple comparison test *P<0.05, **P<0.01, ***P<0.001.

FIG. 11: Fibrotic phenotype in Acod1−/− mice at day 7 and day 21 post bleomycin. (A-B) Gene expression analysis of Col1α1, Col3α1, Col4α1, Fn1 in lung homogenate of PBS and bleomycin dosed WT and Acod1−/− mice at day 7 (A) and day 42 (B) (n=3-6). Actb was used as housekeeping gene. Pooled from two independent experiments. (C) Ashcroft score based on Sirius red staining in lung slices harvested at day 21 post bleomycin (n=11-16 per group; three experiments pooled). Data presented as mean±S.D. Significance was tested by One Way ANOVA+Sidak's multiple comparison test.

FIG. 12: Baseline lung function and immune cell recruitment in WT and Acod1−/− mice. (A-C) Resistance (A), elastance (B) and compliance (C) at baseline measured by FlexiVent in PBS dosed WT and Acod1−/− mice at day 7, day 21 and day 42. (D-F) Total BAL cells (D), numbers of BAL AMs (E) and BAL neutrophils (F) in PBS dosed WT and Acod1−/− mice at day 7, day 21 and day 42. Day 7 both groups n=6, day 21 WT n=7, Acod1−/− n=10; day 42 WT n=7, Acod1−/− n=4; representative of 2 — 3 experiments per time point. Data presented as mean±S.D. Significance was tested by Mann-Whitney-U test per time point.

FIG. 13: Itaconate controls tissue resident AM metabolism. (A) Schematic of dosing regimen using 8-10 week old C57131/6 mice. 0.05U Bleomycin and 0.05 μM PKH26 Celltracker was administered oropharyngeal at indicated time points. (B) Representative FACS plots showing gating strategy to assess PKH-26 Celltracker in Tr-AM and Mo-AM populations (see gating strategy FIG. 9) in murine BAL after PBS or bleomycin treatment. (C) Tr-AM or Mo-AM as % of total AM in BAL of PBS or Bleomycin dosed WT mice at day 7, day 21 and day 42 post bleomycin exposure (n=6-12 per group). (D) Additional read-outs of seahorse mitochondrial stress test (FIG. 3A) of PBS Tr-AM (n=3), Bleo Tr-AM (n=4) and Bleo Mo-AM (n=5), representative of three independent experiments. (E) Representative graph of MitoTracker Green staining in WT and Acod1−/− PBS treated mice in the live, CD45+ fraction and quantification of mean fluorescent intensity (MFI) of WT (n=4) and Acod1−/− (n=5) PBS treated mice. Data presented as mean ±S.D. Significance was tested by ordinary one-way ANOVA with Sidak's multiple comparison test (D) or Mann Whitney U test (F), **P<0.01, ***P<0.001, ****P<0.0005.

FIG. 14: AMs in the adult human lung after transplant express ACOD1. (A) Acod1 expressing AM (CD68) in BAL after male donor (RPS4Y1) to female recipient (XIST) lung transplant. (B) UMAP of Acod1 expressing AMs and monocyte derived macrophages (MDM). (C) Pseudo time analysis of AM and MDM in BAL after male donor to female recipient lung transplant. (D) Pseudo-time analysis of expression of Acod1, KEAP1 and NRF2-target genes NQO1, TALDO1 and HMOX1 in AM and MDM after male donor to female recipient lung transplant.

FIG. 15: Pro-fibrotic genes are increased in Mo-AM compared to Tr-AM after bleomycin exposure (A) Heat map representation of murine fibrosis gene array of sorted Tr-AM and Mo-AM from WT and Acod1−/− 7 days post bleomycin exposure (n=4 for all groups). Data shown as log10 of ΔΔCT WT vs Acod1−/− (B) Volcano plot highlighting differentially expressed genes in WT Mo-AM vs Tr-AM at day 7 post bleomycin exposure. Genes up-regulated are shown in red and those down-regulated in blue. (C) Table highlighting fold change of fibrosis related genes of samples in (A).

FIG. 16: BAL cell composition and collagen gene expression post adoptive transfer. (A-C) Total BAL cells (A), numbers of BAL AMs (B) and BAL neutrophils (C) in PBS or Bleo dosed WT and Acod1−/− mice at day 42; WT-AT n=3, KO-AT n=6. (D) Representative FACS plots of CD11b+/MHC II+ and CD11b-/MHC II-Tr-AM and Mo-AM in BAL of Acod1−/− mice after adoptive transfer of WT or Acod1−/− Mo-AMs; day 21 post bleomycin. WT-AT=adoptive transfer of WT Mo-AMs into Acod1−/− mice, KO-AT=adoptive transfer of Acod1−/− Mo-AM into Acod1−/− mice. Data presented as mean±S.D.; Significance was tested by Mann-Whitney U test.

FIG. 17: Exogenous itaconate limits human lung fibroblast wound healing. (A) Proliferation rate of IPF (n=3) human primary lung fibroblasts stimulated with 10 mM itaconate or vehicle control measured using the JULI Stage system. (B) Wound healing capacity of healthy (n=3) human primary lung fibroblasts stimulated with 10 mM itaconate or vehicle control measured using the JuLI Stage system; (all groups n=4). Two-tailed, unpaired t-test of area under the curve. (C) Representative images of fibroblast wound healing during stimulation with 10 mM itaconate or vehicle control, acquired using the JULI stage system. Statistical significance tested by Mann-Whitney U test of area under the curve, **P<0.01.

FIG. 18: Exogenous itaconate is anti-fibrotic. (A-C) Total BAL count, percentage of eosinophils (B) and Percentage of neutrophils (C) of PBS or itaconate dosed mice (0.25-10 mg/kg, oropharyngeal), 24 h post administration (n=4 per group). Data presented as mean±S.D.; Significance was tested by ordinary one-way ANOVA with Sidak's multiple comparison test, **P<0.01.

### DETAILED DESCRIPTION OF THE INVENTION

### Definitions

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure. The meaning and scope of the terms should be clear; however, in the event of any latent ambiguity, definitions provided herein take precedent over any dictionary or extrinsic definition. It should be understood that this invention is not limited to the particular methodology, protocols, and reagents, etc., described herein and as such can vary.

This disclosure is not limited by the exemplary methods and materials disclosed herein, and any methods and materials similar or equivalent to those described herein can be used in the practice or testing of embodiments of this disclosure. The terminology used herein is for the purpose of describing particular embodiments only, and is not intended to limit the scope of the present invention, which is defined solely by the claims.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure.

As used herein, the term “capable of” when used with a verb, encompasses or means the action of the corresponding verb. For example, “capable of interacting” also means interacting, “capable of cleaving” also means cleaves, “capable of binding” also means binds and “capable of specifically targeting . . . ” also means specifically targets.

Other definitions of terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

Numeric ranges are inclusive of the numbers defining the range. Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

As used herein, the articles “a” and “an” may refer to one or to more than one (e.g. to at least one) of the grammatical object of the article. Further, unless otherwise required by context, singular terms shall include pluralities and plural terms shall include the singular. In this application, the use of “or” means “and/or” unless stated otherwise. Furthermore, the use of the term “including”, as well as other forms, such as “includes” and “included”, is not limiting.

“About” may generally mean an acceptable degree of error for the quantity measured given the nature or precision of the measurements. Exemplary degrees of error are within 20 percent (%), typically, within 10%, and more typically, within 5% of a given value or range of values. Preferably, the term “about” shall be understood herein as plus or minus (±) 5%, preferably ±4%, ±3%, ±2%, ±1%, ±0.5%, ±0.1%, of the numerical value of the number with which it is being used.

The term “consisting of” refers to compositions, methods, and respective components thereof as described herein, which are exclusive of any element not recited in that description of the invention.

As used herein the term “consisting essentially of” refers to those elements required for a given invention. The term permits the presence of elements that do not materially affect the basic and novel or functional characteristic(s) of that invention (i.e. inactive or non-immunogenic ingredients).

Embodiments described herein as “comprising” one or more features may also be considered as disclosure of the corresponding embodiments “consisting of” and/or “consisting essentially of” such features.

Concentrations, amounts, volumes, percentages and other numerical values may be presented herein in a range format. It is also to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

Amino acids are referred to herein using the name of the amino acid, the three-letter abbreviation or the single letter abbreviation.

As used herein, the terms “protein” and “polypeptide” are used interchangeably herein to designate a series of amino acid residues, connected to each other by peptide bonds between the alpha-amino and carboxyl groups of adjacent residues. The terms “protein”, and “polypeptide” refer to a polymer of amino acids, including modified amino acids (e.g., phosphorylated, glycated, glycosylated, etc.) and amino acid analogues, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogues of the foregoing.

Minor variations in the amino acid sequences of proteins of the invention are contemplated as being encompassed by the present invention, providing that the variations in the amino acid sequence(s) maintain at least 60%, at least 70%, more preferably at least 80%, at least 85%, at least 90%, at least 95%, and most preferably at least 97% or at least 99% sequence identity to the proteins of the invention or an immunogenic fragment thereof as defined anywhere herein. The term homology is used herein to mean identity. As such, the sequence of a variant or analogue sequence of a protein of the invention may differ on the basis of substitution (typically conservative substitution) deletion or insertion.

Proteins of the invention may include variants in which amino acid residues from one species are substituted for the corresponding residue in another species, either at the conserved or non-conserved positions. Variants of protein molecules disclosed herein may be produced and used in the present invention. Following the lead of computational chemistry in applying multivariate data analysis techniques to the structure/property-activity relationships [see for example, Wold, et al. Multivariate data analysis in chemistry. Chemometrics-Mathematics and Statistics in Chemistry (Ed.: B. Kowalski); D. Reidel Publishing Company, Dordrecht, Holland, 1984 (ISBN 90-277-1846-6] quantitative activity-property relationships of proteins can be derived using well-known mathematical techniques, such as statistical regression, pattern recognition and classification [see for example Norman et al. Applied Regression Analysis. Wiley-Interscience; 3rd edition (April 1998) ISBN: 0471170828; Kandel, Abraham et al. Computer-Assisted Reasoning in Cluster Analysis. Prentice Hall PTR, (May 11, 1995), ISBN: 0133418847; Krzanowski, Wojtek. Principles of Multivariate Analysis: A User's Perspective (Oxford Statistical Science Series, No 22 (Paper)). Oxford University Press; (December 2000), ISBN: 0198507089; Witten, Ian H. et al Data Mining: Practical Machine Learning Tools and Techniques with Java Implementations. Morgan Kaufmann; (Oct. 11, 1999), ISBN:1558605525; Denison David G. T. (Editor) et al Bayesian Methods for Nonlinear Classification and Regression (Wiley Series in Probability and Statistics). John Wiley & Sons; (July 2002), ISBN: 0471490369; Ghose, Arup K. et al. Combinatorial Library Design and Evaluation Principles, Software, Tools, and Applications in Drug Discovery. ISBN: 0-8247-0487-8]. The properties of proteins can be derived from empirical and theoretical models (for example, analysis of likely contact residues or calculated physicochemical property) of proteins sequence, functional and three-dimensional structures and these properties can be considered individually and in combination.

Amino acids are referred to herein using the name of the amino acid, the three-letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Amino acid residues at non-conserved positions may be substituted with conservative or non-conservative residues. In particular, conservative amino acid replacements are contemplated.

A “conservative amino acid substitution” is one in which the amino acid residue is replaced with an amino acid residue having a similar side chain. Families of amino acid residues having similar side chains have been defined in the art, including basic side chains (e.g., lysine, arginine, or histidine), acidic side chains (e.g., aspartic acid or glutamic acid), uncharged polar side chains (e.g., glycine, asparagine, glutamine, serine, threonine, tyrosine, or cysteine), nonpolar side chains (e.g., alanine, valine, leucine, isoleucine, proline, phenylalanine, methionine, or tryptophan), beta-branched side chains (e.g., threonine, valine, isoleucine) and aromatic side chains (e.g., tyrosine, phenylalanine, tryptophan, or histidine). Thus, if an amino acid in a polypeptide is replaced with another amino acid from the same side chain family, the amino acid substitution is considered to be conservative. The inclusion of conservatively modified variants in a protein of the invention does not exclude other forms of variant, for example polymorphic variants, interspecies homologs, and alleles.

“Non-conservative amino acid substitutions” include those in which (i) a residue having an electropositive side chain (e.g., Arg, His or Lys) is substituted for, or by, an electronegative residue (e.g., Glu or Asp), (ii) a hydrophilic residue (e.g., Ser or Thr) is substituted for, or by, a hydrophobic residue (e.g., Ala, Leu, Ile, Phe or Val), (iii) a cysteine or proline is substituted for, or by, any other residue, or (iv) a residue having a bulky hydrophobic or aromatic side chain (e.g., Val, His, Ile or Trp) is substituted for, or by, one having a smaller side chain (e.g., Ala or Ser) or no side chain (e.g., Gly).

“Insertions” or “deletions” are typically in the range of about 1, 2, or 3 amino acids. The variation allowed may be experimentally determined by systematically introducing insertions or deletions of amino acids in a protein using recombinant DNA techniques and assaying the resulting recombinant variants for activity. This does not require more than routine experiments for a skilled person.

A “fragment” of a polypeptide comprises at least 50%, at least 60%, at least 70%, at least 80%, at least 90%, at least 95%, at least 97% or more of the original polypeptide.

The proteins of the invention, or immunogenic fragments thereof, include both intact and modified forms of the proteins disclosed herein. For example, a protein of the invention or immunogenic fragment thereof can be functionally linked (e.g. by chemical coupling, genetic fusion, noncovalent association, or otherwise) to one or more other molecular entities, such as a pharmaceutical agent, a detection agent, and/or a protein or peptide that can mediate association of a binding molecule disclosed herein with another molecule (e.g. a streptavidin core region or a polyhistidine tag) Non-limiting examples of detection agents include: enzymes, such as alkaline phosphatase, glucose-6-phosphate dehydrogenase (“G6PDH”), alpha-D-galactosidase, glucose oxydase, glucose amylase, carbonic anhydrase, acetylcholinesterase, lysozyme, malate dehydrogenase and peroxidase, e.g., horseradish peroxidase; dyes; fluorescent labels or fluorescers, such as fluorescein and its derivatives, fluorochrome, rhodamine compounds and derivatives, GFP (GFP for “Green Fluorescent Protein”), dansyl, umbelliferone, phycoerythrin, phycocyanin, allophycocyanin, o-phthaldehyde, and fluorescamine; fluorophores such as lanthanide cryptates and chelates, e.g., Europium etc., (Perkin Elmer and Cis Biointernational); chemoluminescent labels or chemiluminescers, such as isoluminol, luminol and the dioxetanes; bio-luminescent labels, such as luciferase and luciferin; sensitizers; coenzymes; enzyme substrates; radiolabels, including but not limited to, bromine⁷⁷, carbon¹⁴, cobalt⁵⁷, fluorine⁸, gallium⁶⁷, gallium⁶⁸, hydrogen³(tritium), indium¹¹¹, indium^{113m}, iodine^{123m}, iodine¹²⁵, iodine¹²⁶, iodine¹³¹, iodin¹³³, mercury^{107,} mercury²⁰³, phosphorous³², rhenium^{99m}, rhenium¹⁰¹, rhenium¹⁰⁵, ruthenium⁹⁵, ruthenium⁹⁷, ruthenium¹⁰³, ruthenium¹⁰⁵, scandium⁴⁷, selenium⁷⁵, sulphur³⁵, technetium⁹⁹, technetium^{99m}, tellurium^{121m}, tellurium^{122m}, tellurium^{125m}, thulium¹⁶⁵, thulium¹⁶⁷, thulium¹⁶⁸ and yttrium¹⁹⁹; particles, such as latex or carbon particles, metal sol, crystallite, liposomes, cells, etc., which may be further labelled with a dye, catalyst or other detectable group; molecules such as biotin, digoxygenin or 5-bromodeoxyuridine; toxin moieties, such as for example a toxin moiety selected from a group of Pseudomonas exotoxin (PE or a cytotoxic fragment or mutant thereof), Diptheria toxin or a cytotoxic fragment or mutant thereof, a Botulinum toxin A, B, C, D, E or F, ricin or a cytotoxic fragment thereof e.g. ricin A, abrin or a cytotoxic fragment thereof, saporin or a cytotoxic fragment thereof, pokeweed antiviral toxin or a cytotoxic fragment thereof and bryodin 1 or a cytotoxic fragment thereof.

The proteins of the invention or immunogenic fragments thereof also include derivatives that are modified (e.g., by the covalent attachment of any type of molecule to the protein) such that covalent attachment does not prevent the protein from binding to antibodies specific for said protein, or otherwise impair the biological activity of the protein. Examples of suitable derivatives include, but are not limited to fucosylated proteins, glycosylated proteins, acetylated proteins, PEGylated proteins, phosphorylated proteins, and amidated proteins.

A typical antibody comprises at least two “light chains” (LC) and two “heavy chains” (HC). The light chains and heavy chains of such antibodies are polypeptides consisting of several domains. Each heavy chain comprises a heavy chain variable region (abbreviated herein as “VH”) and a heavy chain constant region (abbreviated herein as “CH”). The heavy chain constant region comprises the heavy chain constant domains CH1, CH2 and CH3 (antibody classes IgA, IgD, and IgG) and optionally the heavy chain constant domain CH4 (antibody classes IgE and IgM). Each light chain comprises a light chain variable domain (abbreviated herein as “VL”) and a light chain constant domain (abbreviated herein as “CL”). The variable regions VH and VL can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The “constant domains” of the heavy chain and of the light chain are not involved directly in binding of an antibody to a target, but exhibit various effector functions.

Binding between an antibody and its target antigen or epitope is mediated by the Complementarity Determining Regions (CDRs). The CDRs are regions of high sequence variability, located within the variable region of the antibody heavy chain and light chain, where they form the antigen-binding site. The CDRs are the main determinants of antigen specificity. Typically, the antibody heavy chain and light chain each comprise three CDRs which are arranged non-consecutively. The antibody heavy and light chain CDR3 regions play a particularly important role in the binding specificity/affinity of the antibodies according to the invention and therefore provide a further aspect of the invention.

Thus, the term “antigen binding fragment” as used herein incudes any naturally-occurring or artificially-constructed configuration of an antigen-binding polypeptide comprising one, two or three light chain CDRs, and/or one, two or three heavy chain CDRs, wherein the polypeptide is capable of binding to the antigen.

The sequence of a CDR may be identified by reference to any number system known in the art, for example, the Kabat system (Kabat, E. A., et al., Sequences of Proteins of Immunological Interest, 5th ed., Public Health Service, National Institutes of Health, Bethesda, MD (1991); the Chothia system (Chothia &, Lesk, “Canonical Structures for the Hypervariable Regions of Immunoglobulins,” J. Mol. Biol. 196, 901-917 (1987)); or the IMGT system (Lefranc et al., “IMGT Unique Numbering for Immunoglobulin and Cell Receptor Variable Domains and Ig superfamily V-like domains,” Dev. Comp. Immunol. 27,55-77 (2003)).

For heavy chain constant region amino acid positions discussed in the invention, numbering is according to the EU index first described in Edelman, G. M., et al., Proc. Natl. Acad. Sci. USA 63 (1969) 78-85). The EU numbering of Edelman is also set forth in Kabat et al. (1991) (supra.). Thus, the terms “EU index as set forth in Kabat”, “EU Index”. “EU index of Kabat” or “EU numbering” in the context of the heavy chain refers to the residue numbering system based on the human IgG1 EU antibody of Edelman et al. as set forth in Kabat et al. (1991). The numbering system used for the light chain constant region amino acid sequence is similarly set forth in Kabat et al. (supra.). Thus, as used herein, “numbered according to Kabat” refers to the Kabat numbering system set forth in Kabat et al. (supra.).

The antibodies of the invention or antigen-binding fragments thereof are preferably monoclonal antibodies. More preferably, the antibodies of the invention or antigen-binding fragments thereof are isolated monoclonal antibodies.

The term “humanized antibody” refers to antibodies in which the framework or “complementarity determining regions” (CDRs) have been modified to comprise the CDR of an immunoglobulin of different specificity as compared to that of the parent immunoglobulin. For example, a murine CDR may be grafted into the framework region of a human antibody to prepare the “humanized antibody.” See, e.g., Riechmann, L., et al., Nature 332 (1988) 323-327; and Neuberger, M. S., et al., Nature 314 (1985) 268-270. In some embodiments, “humanized antibodies” are those in which the constant region has been additionally modified or changed from that of the original antibody to generate the properties of the antibodies according to the invention, especially in regard to Clq binding and/or Fc receptor (FcR) binding.

The term “chimeric antibody” refers to an antibody comprising a variable region, i.e., binding region, from one source or species and at least a portion of a constant region derived from a different source or species, usually prepared by recombinant DNA techniques. Chimeric antibodies comprising a murine variable region and a human constant region are preferred. Other preferred forms of “chimeric antibodies” encompassed by the present invention are those in which the constant region has been modified or changed from that of the original antibody to generate the properties of the antibodies according to the invention, especially in regard to Clq binding and/or Fc receptor (FcR) binding. Such chimeric antibodies are also referred to as “class-switched antibodies”. Chimeric antibodies are the product of expressed immunoglobulin genes comprising DNA segments encoding immunoglobulin variable regions and DNA segments encoding immunoglobulin constant regions. Methods for producing chimeric antibodies involving conventional recombinant DNA and gene transfection techniques are well known in the art. See, e.g., Morrison, S. L., et al., Proc. Natl. Acad. Sci. USA 81 (1984) 6851-6855; U.S. Pat. Nos. 5,202,238 and 5,204,244.

The terms “Fc region”, “Fc part” and “Fc” are used interchangeably herein and refer to the portion of a native immunoglobulin that is formed by two Fc chains. Each “Fc chain” comprises a constant domain CH2 and a constant domain CH3. Each Fc chain may also comprise a hinge region. A native Fc region is homodimeric. In some embodiments, the Fc region may be heterodimeric because it may contain modifications to enforce Fc heterodimerization.

There are five major classes of heavy chain constant region, classified as IgA, IgG, IgD, IgE and IgM, each with characteristic effector functions designated by isotype. For example, IgG is separated into four subclasses known as IgG1, IgG2, IgG3, and IgG4. Ig molecules interact with multiple classes of cellular receptors. For example, IgG molecules interact with three classes of Fcy receptors (FcγR) specific for the IgG class of antibody, namely FcγRI, FcγRII, and FcγIII. The important sequences for the binding of IgG to the FcγR receptors have been reported to be located in the CH2 and CH3 domains.

The antibodies of the invention or antigen-binding fragments thereof may be any isotype, i.e. IgA, IgD, IgE, IgG and IgM, and synthetic multimers of the four-chain immunoglobulin (Ig) structure.

The terms “Fab fragment” and “Fab” are used interchangeably herein and contain a single light chain (e.g. a constant domain CL and a VL) and a single heavy chain (e.g. the constant domain CH1 and a VH). The heavy chain of a Fab fragment is not capable of forming a disulfide bond with another heavy chain.

A “Fab′ fragment” contains a single light chain and a single heavy chain but in addition to the CH1 and the VH, a “Fab′ fragment” contains the region of the heavy chain between the CH1 and CH2 domains that is required for the formation of an inter-chain disulfide bond. Thus, two “Fab′ fragments” can associate via the formation of a disulphide bond to form a F(ab′)2 molecule.

A “F(ab′)2 fragment” contains two light chains and two heavy chains. Each chain includes a portion of the constant region necessary for the formation of an inter-chain disulfide bond between two heavy chains.

An “Fv fragment” contains only the variable regions of the heavy and light chain. It contains no constant regions.

A “single-domain antibody” is an antibody fragment containing a single antibody domain unit (e.g., VH or VL).

A “single-chain Fv” (“scFv”) is antibody fragment containing the VH and VL domain of an antibody, linked together to form a single chain. A polypeptide linker is commonly used to connect the VH and VL domains of the scFv.

A “tandem scFv”, also known as a TandAb®, is a single-chain Fv molecule formed by covalent bonding of two scFvs in a tandem orientation with a flexible peptide linker.

A “bi-specific T cell engager” (BiTE®) is a fusion protein consisting of two single-chain variable fragments (scFvs) on a single peptide chain. One of the scFvs binds to T cells via the CD3 receptor, and the other to a tumour cell antigen.

A “diabody” is a small bivalent and bispecific antibody fragment comprising a heavy (VH) chain variable domain connected to a light chain variable domain (VL) on the same polypeptide chain (VH-VL) connected by a peptide linker that is too short to allow pairing between the two domains on the same chain (Kipriyanov, Int. J. Cancer 77 (1998), 763-772). This forces pairing with the complementary domains of another chain and promotes the assembly of a dimeric molecule with two functional antigen binding sites.

The antibodies of the invention or antigen-binding fragments thereof also include derivatives that are modified (e.g., by the covalent attachment of any type of molecule to the antibody) such that covalent attachment does not prevent the antibody from binding to its epitope, or otherwise impair the biological activity of the antibody. Examples of suitable derivatives include, but are not limited to fucosylated antibodies, glycosylated antibodies, acetylated antibodies, PEGylated antibodies, phosphorylated antibodies, and amidated antibodies.

Further embodiments are multispecific antibodies (bispecific, trispecific etc.) and other conjugates, e.g. with cytotoxic small molecules.

As used herein, the terms “polynucleotides”, “nucleic acid” and “nucleic acid sequence” refers to any molecule, preferably a polymeric molecule, incorporating units of ribonucleic acid, deoxyribonucleic acid or an analogue thereof. The nucleic acid can be either single-stranded or double-stranded. A single-stranded nucleic acid can be one nucleic acid strand of a denatured double-stranded DNA Alternatively, it can be a single-stranded nucleic acid not derived from any double-stranded DNA. In one aspect, the nucleic acid can be DNA. In another aspect, the nucleic acid can be RNA Suitable nucleic acid molecules are DNA, including genomic DNA or cDNA. Other suitable nucleic acid molecules are RNA, including siRNA, shRNA, and antisense oligonucleotides.

An exemplary, but non-limiting amino acid sequence of SDHA may comprise or consist of UniProt Accession No. P31040 (version 2 of the sequence, entry last modified 12 Aug. 2020, provided herein as SEQ ID NO: 1). The corresponding nucleic acid sequence is SEQ ID NO: 2.

An exemplary, but non-limiting amino acid sequence of SDHB may comprise or consist of UniProt Accession No. P21912 (version 3 of the sequence, entry last modified 12 Aug. 2020, provided herein as SEQ ID NO: 3). The corresponding nucleic acid sequence is SEQ ID NO: 4.

An exemplary, but non-limiting amino acid sequence of SDHC may comprise or consist of UniProt Accession No. Q99643 (version 1 of the sequence, entry last modified 12 Aug. 2020, provided herein as SEQ ID NO: 5). The corresponding nucleic acid sequence is SEQ ID NO: 6.

An exemplary, but non-limiting amino acid sequence of SDHD may comprise or consist of UniProt Accession No. 014521 (version 1 of the sequence, entry last modified 12 Aug. 2020, provided herein as SEQ ID NO: 7). The corresponding nucleic acid sequence is SEQ ID NO: 8.

An exemplary, but non-limiting amino acid sequence of ACOD1 may comprise or consist of UniProt Accession No. A6NK06 (version 1 of the sequence, entry last modified 12 Aug. 2020, provided herein as SEQ ID NO: 9). The corresponding nucleic acid sequence is SEQ ID NO: 10.

The terms “decrease”, “reduced”, “reduction”, or “inhibit” are all used herein to mean a decrease by a statistically significant amount. The terms “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99% , or more. As used herein, “reduction” or “inhibition” encompasses a complete inhibition or reduction as compared to a reference level. “Complete inhibition” is a 100% inhibition (i.e. abrogation_as compared to a reference level. A decrease can be preferably down to a level accepted as within the range of normal for an individual without a given disorder.

The terms “increased”, “increase”, “enhance”, or “activate” are all used herein to mean an increase by a statically significant amount. The terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

References herein to the level of a particular molecule (e.g. itaconate or any of the other agents described herein) encompass the actual amount of the molecule, such as the mass, molar amount, concentration or molarity of the molecule. Preferably in the context of the invention, references to the level of a particular molecule refer to the concentration of the molecule.

The level of a molecule may be determined in any appropriate physiological compartment. Preferred physiological compartments include bronchoalveolar lavage (BAL), plasma, whole blood and/or serum. The level of a molecule may be determined from any appropriate sample from an individual, e.g. a BAL sample plasma sample, a blood sample and/or a serum sample. Other non-limiting examples of samples which may be tested are tissue or fluid samples urine and biopsy samples. Thus, by way of non-limiting example, the invention may reference the level (e.g. concentration) of a molecule (e.g. itaconate) in the BAL and/or plasma of an individual. The level of a molecule pre-treatment with an agent of the invention may be interchangeably referred to as the “baseline”.

The level of a molecule (e.g. itaconate, collagen, fibrinogen and/or hydroxyproline) may be compared with any appropriate control. For example, a control may be obtained from a healthy individual or an individual without (clinically relevant) fibrosis in the tissue to be treated according to the invention. Alternatively, the control may be obtained from the same individual prior to treatment, or from a different individual with (clinically relevant) fibrosis in the same tissue type as to be treated, but wherein the different individual has not been treated with the agent, vector, composition, drug delivery system or Mo-M population of the invention.

The level of a molecule (e.g. itaconate, collagen, fibrinogen and/or hydroxyproline) after treatment with an agent of the invention may be compared with the level of the molecule in the individual pre-treatment with the agent. Thus, the invention may be concerned with the relative level of the molecule (e.g. itaconate, collagen, fibrinogen and/or hydroxyproline) pre- and post-treatment. The level of a molecule pre-treatment (e.g. itaconate, collagen, fibrinogen and/or hydroxyproline) may be used to identify an individual as suitable for treatment according to the invention. Other parameters may also be used, either alone or in combination with the level of a moiecule as described above, to identify an individual as suitable for treatment according to the invention. Suitable parameters to identify an individual as suitable for treatment according to the invention are known to the skilled person. Typically, the presence and/or amount of a biomarker is used to identify an individual as suitable for treatment. The biomarker may, for example, be a circuiating protein biomarker. The circulating biomarker may be a epithelil cell damage marker (e.g. cytokeratin 19 fragment (CYFRA 21-1) or carbohydrate antigen 125 (CA125)), a marker of collagen turnover (e.g. procollagen type I N-terminal propeptide (PINP); procollagen type I C-terminal propeptide (PICP); carboxyl-terminal peptide of procollagen type I (PIP); and carboxyl-terminal telopeptide of collagen type I (CITP)), or a marker ofmacrophage function (e.g. YKI40, CCL18 and PAI1). Imaging biomarkers may also be used to identify an individual as suitable for treatment according to the invention The imaging biomarkers may be identified, for example, through analysis of computerised tomography (CT) images.

The level of a molecule may be measured directly or indirectly, and may be determined using any appropriate technique. Suitable standard techniques are known in the art, for example Western blotting and enzyme-linked immunosorbent assays (ELISAs).

An individual can be one who has been previously diagnosed with or identified as suffering from or having a condition in need of treatment or one or more complications related to such a condition, and optionally, have already undergone treatment for a condition as defined herein or the one or more complications related to said condition. Alternatively, an individual can also be one who has not been previously diagnosed as having a condition as defined herein or one or more complications related to said condition. For example, an individual can be one who exhibits one or more risk factors for a condition, or one or more complications related to said condition or a subject who does not exhibit risk factors.

An “individual in need” of treatment for a particular condition can be an individual having that condition, diagnosed as having that condition, or at risk of developing that condition.

The terms “subject”, “individual” and “patient” are used interchangeably herein to refer to a mammalian individual. An “individual” may be any mammal. Generally, the individual may be human; in other words, in one embodiment, the “individual” is a human. A “individual” may be an adult, juvenile or infant. An “individual” may be male or female.

The term “pharmaceutically acceptable” as used herein means approved by a regulatory agency of the Federal or a state government, or listed in the U.S. Pharmacopeia, European Pharmacopeia or other generally recognized pharmacopeia

An “analogue” of a chemical structure, as the term is used herein, refers to a chemical structure that preserves substantial similarity with the parent structure, although it may not be readily derived synthetically from the parent structure. A related chemical structure that is readily derived synthetically from a parent chemical structure is referred to as a “derivative.”

A “hydrate” is a compound that exists in a composition with water molecules. The composition can include water in stoichiometric quantities, such as a monohydrate or a dihydrate, or can include water in random amounts. As the term is used herein a “hydrate” refers to a solid form, i.e., a compound in water solution, while it may be hydrated, is not a hydrate as the term is used herein.

A “solvate” is a similar composition except that a solvent other that water replaces the water. For example, methanol or ethanol can form an “alcoholate”, which can again be stoichiometric or non-stoichiometric. As the term is used herein a “solvate” refers to a solid form, i.e., a compound in solution in a solvent, while it may be solvated, is not a solvate as the term is used herein.

A “prodrug” as is well known in the art is a substance that can be administered to an individual where the substance is converted in vivo by the action of biochemicals within the patient's body, such as enzymes, to the active pharmaceutical ingredient. Examples of prodrugs include esters of carboxylic acid groups, which can be hydrolyzed by endogenous esterases as are found in the bloodstream of humans and other mammals. Conventional procedures for the selection and preparation of suitable prodrug derivatives are described, for example, in “Design of Prodrugs”, ed. H. Bundgaard, Elsevier, 1985.

A “salt” as is well known in the art includes an organic compound such as a carboxylic acid, a sulfonic acid, or an amine, in ionic form, in combination with a counterion. For example, acids in their anionic form can form salts with cations such as metal cations, for example sodium, potassium, and the like; with ammonium salts such as NH4+ or the cations of various amines, including tetraalkyl ammonium salts such as tetramethylammonium, or other cations such as trimethylsulfonium, and the like. A “pharmaceutically acceptable” or “pharmacologically acceptable” salt is a salt formed from an ion that has been approved for human consumption and is generally non-toxic, such as a chloride salt or a sodium salt. A “zwitterion” is an internal salt such as can be formed in a molecule that has at least two ionisable groups, one forming an anion and the other a cation, which serve to balance each other. For example, amino acids such as glycine can exist in a zwitterionic form. A “zwitterion” is a salt within the meaning herein. The compounds of the present invention may take the form of salts. The term “salts” embraces addition salts of free acids or free bases which are compounds of the invention. Salts can be “pharmaceutically-acceptable salts.” The term “pharmaceutically-acceptable salt” refers to salts which possess toxicity profiles within a range that affords utility in pharmaceutical applications.

Pharmaceutically unacceptable salts may nonetheless possess properties such as high crystallinity, which have utility in the practice of the present invention, such as for example utility in process of synthesis, purification or formulation of compounds of the invention.

Suitable pharmaceutically-acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid. Examples of pharmaceutically unacceptable acid addition salts include, for example, perchlorates and tetrafiuoroborates.

Suitable pharmaceutically acceptable base addition salts of compounds of the invention include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N-dibenzylethylenediamine, chloroprocaine, choline, diethanolamine, ethylenediamine, meglumine (N-methylglucamine) and procaine. Examples of pharmaceutically unacceptable base addition salts include lithium salts and cyanate salts. Although pharmaceutically unacceptable salts are not generally useful as medicaments, such salts may be useful, for example as intermediates in the synthesis of Formula (I) compounds, for example in their purification by recrystallization. All of these salts may be prepared by conventional means from the corresponding compound according to Formula (I) by reacting, for example, the appropriate acid or base with the compound according to Formula (I). The term “pharmaceutically acceptable salts” refers to nontoxic inorganic or organic acid and/or base addition salts, see, for example, Lit et al., Salt Selection for Basic Drugs (1986), Int J. Pharm., 33, 201-217, incorporated by reference herein.

The publications discussed herein are provided solely for their disclosure prior to the filing date of the present application. Nothing herein is to be construed as an admission that such publications constitute prior art to the claims appended hereto.

### Inhibition of Succinate Dehydrogenase

Succinate dehydrogenase (SDH) is a heterotetrameric enzyme complex that catalyses the sixth step of the tricarboxylic acid cycle (TCA), i.e. the dehydrogenation of succinate to fumarate. This reaction, through the generation of electrons, couples the TCA with the electron transfer chain. The four subunits are encoded by four genes SDHA, SDHB, SDHC and SDHD. SDHA encodes the main catalytic subunit, a flavoprotein (Fp) containing oxidoreductase.

The terms “inhibit SDH”, “inhibition of SDH” and “SDH inhibitor” as used herein relate to inhibition of the catalytic activity of SDH, and can be used interchangeably with the terms “inhibit SDHA”, “inhibition of SDHA” and “SDHA inhibitor”.

The present invention provides agents which inhibit SDH for use in the treatment or prevention of tissue fibrosis. Such agents and associated vectors, compositions and drug delivery systems are described herein. Also described are monocyte-recruited macrophage (Mo-M) populations which use the same underlying mechanisms and can also be used in the treatment or prevention of tissue fibrosis.

The invention relates to both direct and indirect inhibition of SDH. Unless explicitly stated, references herein to inhibition of SDH encompass both direct and indirect inhibition of SDH. In some preferred embodiments, the invention relates to direct inhibition of SDH.

“Direct inhibition of SDH” as used herein means inhibition of the expression and/or activity of SDH directly, i.e. without any intermediary step. By way of non-limiting example, direct inhibition of SDH may elicited by competitive or non-competitive inhibitors of the SDH enzyme or by inhibition of a gene or genes encoding the subunits of the SDH enzyme.

“Indirect inhibition of SDH” as used herein means inhibition of the expression and/or activity of SDH indirectly, i.e. through the modulation or delivery of genes/enzymes upstream of SDH and/or through the generation or delivery of intermediaries which directly inhibit SDH. Indirect inhibition may be elicited by upregulating the expression of an enzyme which generates an endogenous direct inhibitor of SDH. By way of non-limiting example, indirect inhibition of SDH may involve increasing the expression and/or activity of aconitate decarboxylase 1 (ACOD1). ACOD1 encodes cis-aconitate decarboxylase (CAD), which catalyses the decarboxylation of cis-aconitate to itaconate. The itaconate produced inhibits SDH. The degree of indirect inhibition may be as defined above.

Expression may be quantified in terms of gene and/or protein expression, and may be compared with the expression of a control (e.g. housekeeping gene or protein). As a non-limiting example, in the context of SDH expression, the actual amount of an SDH gene, mRNA transcript and/or protein, such as the mass, molar amount, concentration or molarity of an SDH gene, mRNA transcript and/or protein, or the number of mRNA molecules per cell in a sample obtained from an individual treated according to the invention and the control may be assessed, and compared with the corresponding value from the control. Alternatively, the expression of an SDH gene and/or protein in a sample obtained from an individual treated according to the invention may be compared with that of the control without quantifying the mass, molar amount, concentration or molarity of the one or more gene and/or protein.

Typically, the control is an equivalent sample in which no inhibition of SDH expression has been effected. As a non-limiting example, in the case where an individual is treated with an agent that inhibits SDH expression, a suitable control would be a different individual to which the compound has not been administered or the same individual prior to administration of the compound. Conventional methods for the assessment of gene and/or protein expression are well known in the art and include RT-qPCR, ELISA, DNA microarray, RNA Seq, serial analysis of gene expression (SAGE) and western blotting.

SDH activity may be quantified in terms of the enzyme's consumption of substrate or production of product, and may be compared with the activity of a control (i.e. recombinant enzyme of known concentration). Conventional methods for the assessment of SDH activity are known in the art and include colorimetric and fluorometric assays.

In the context of the present invention, when referring to (direct or indirect) inhibition of SDH expression and/or activity, the degree of inhibition may be as defined above. Typically, inhibition of SDH resulting in a decrease in SDH activity and/or expression of at least about 5%, at least about 10%, preferably at least about 20%, at least about 25%, at least about 30%, at least about 50%, at least about 75%, up to complete inhibition or abrogation of SDH activity and/or expression.

### Agents Which Inhibit SDH

An agent which inhibits SDH according to the invention may directly or indirectly inhibit SDH as described herein.

An agent which inhibits SDH according to the invention may selectively inhibit SDH. This is typically the case for agents which directly inhibit SDH. For such agents which directly inhibit SDH, selectivity may mean that the agent binds selectively (also referred to interchangeably herein as specifically) with SDH. By “binds selectively”, it will be understood that said agent binds to SDH, with no significant cross-reactivity to any other molecule. Cross-reactivity may be assessed by any suitable method. By way of non-limiting example, cross-reactivity of an agent which inhibits SDH with a molecule other than SDH may be considered significant if the agent binds to the other molecule at least 5%, 10%, 15%, 20%, 25%, 30%, 35%, 40%, 45%, 50%, 55%, 60%, 65%, 70%, 75%, 80%, 85%, 90% or 100% as strongly as it binds to SDH. An agent that directly inhibits SDH and that binds selectively to SDH may bind to another molecule at less than 90%, 85%, 80%, 75%, 70%, 65%, 60%, 55%, 50%, 45%, 40%, 35%, 30%, 25% or 20% the strength that it binds to SDH. Preferably, the agent binds to the other molecule at less than 20%, less than 15%, less than 10% or less than 5%, less than 2% or less than 1% the strength that it binds to SDH.

Any suitable agent which inhibits SDH may be used according to the present invention. Non-limiting examples of suitable agents include small molecules, antibodies and antigen-binding fragments thereof, peptides and peptidomimetics, nucleic acids and aptamers, as described herein.

An agent which directly inhibits SDH may be selected from a small molecule, a nucleic acid (for example, an siRNA, shRNA, or antisense oligonucleotide), antibody or antigen-binding fragment, or an aptamer. Preferably an agent which directly inhibits SDH is a small molecule.

An agent which indirectly inhibits SDH may be selected from a small molecule, a nucleic acid (for example, an siRNA, shRNA, or antisense oligonucleotide), antibody or antigen-binding fragment, or an aptamer.

Preferably an agent that indirectly inhibits SDH increases the expression and/or activity of ACOD1. In the context of the present invention, when referring to increasing the expression and/or activity of ACOD1, the degree of increase may be as defined above. Typically, increasing the expression and/or activity of ACOD1 refers to an increase in ACOD1 expression and/or activity of at least about 50%, or at least about 60%, or at least about 70%, or at least about 80%, or at least about 90%, at least about 100% or more. The disclosure herein regarding determining and/or quantifying the expression and or activity of SDH can also be applied in the context of ACOD1. By way of non-limiting example, ACOD1 activity may be quantified in terms of the enzyme's consumption of substrate or production of product, and may be compared with the activity of a control (i.e. recombinant enzyme of known concentration). Conventional methods for the assessment of ACOD1 activity are known in the art and include colorimetric and fluorometric assays.

An agent of the invention may result in an increase in the level of itaconate within the tissue to be treated. In this context, the level of itaconate encompasses, the actual amount of itaconate, such as the mass, molar amount, concentration or molarity of itaconate (for a set sample size or in individual cells of said sample). Typically, the level of itaconate is determined in a sample obtained from an individual treated according to the invention and the control may be assessed quantitatively, and compared with the corresponding value from the control. Alternatively, the level of itaconate in a sample obtained from an individual treated according to the invention may be compared qualitatively with that of the control i.e. without quantifying the mass, molar amount, concentration or molarity of itaconate.

A combination of agents may be used to inhibit SDH. By way of non-limiting examples, a combination of agents may comprise: a direct inhibitor of SDH and an indirect inhibitor of SDH; at least two direct inhibitors of SDH; or at least two indirect inhibitors of SDH.

### Small Molecules

Small molecules may be used as agents which inhibit SDH as described herein. In some embodiments, small molecule agents which inhibit SDH are preferred.

As defined herein, small molecules are low molecular weight compounds, typically organic compounds. Typically, a small molecule has a maximum molecular weight of 900 g/mol, allowing for rapid diffusion across cell membranes. In some embodiments, the maximum molecular weight of a small molecule is 500 g/mol. Typically, a small molecule has a size in the order of 1nm.

Standard techniques are known in the art for the production of small molecules, which can then readily be tested for the ability to inhibit SDH as described herein.

Examples of small molecule agents which (directly and/or indirectly) inhibit SDH include itaconate (described in more detail herein) dimethyl malonate, malonate and oxaloacetate.

It will be understood that when small molecule agents of the present invention contain one or more chiral centres, the compounds may exist in, and may be isolated as pure enantiomeric or diastereomeric forms or as racemic mixtures. The present invention therefore includes any possible enantiomers, diastereomers, racemates or mixtures thereof of small molecule agents of the invention.

Small molecule agents of the present invention may have rotameric forms, or may not have rotational activity. Rotameric forms include slow rotating forms and fast rotating forms. In some preferred embodiments, fast rotating forms of the small molecule agents of the present invention are preferred.

A small molecule agent or a salt thereof may exhibit the phenomenon of tautomerism whereby two chemical compounds that are capable of facile interconversion by exchanging a hydrogen atom between two atoms, to either of which it forms a covalent bond. Since the tautomeric compounds exist in mobile equilibrium with each other they may be regarded as different isomeric forms of the same compound. The invention encompasses any tautomeric form of a small molecule agent, and is not to be limited merely to any one tautomeric form. Thus, small molecule agents according to the invention encompass tautomers (including keto-enol and amide-imidic acid forms).

Small molecule agents may be used in the form of pro-drugs which convert into active small molecule agents in the body, analogues or derivates, as well as in salt, hydrate and solvate forms, as defined in the Definitions section herein.

### Itaconate

The present inventors are the first to discover the anti-fibrotic effects of itaconate. Accordingly, in some preferred embodiments of the invention, the small molecule agent which inhibits SDH is itaconate or a derivative or analogue thereof. The itaconate, or derivative or analogue thereof, may also be in the form of a pharmaceutically acceptable salt.

The term “itaconate” as used herein refers to 2-methylidenebutanedioic acid as a well as derivatives or analogues thereof. Various synonyms of itaconate are known to the skilled person including, itaconic acid, 2-methylenesuccinic acid, 2-propene-1,2-dicarboxylic acid, methylenebutanedioic acid, methylenesuccinic acid, and propylenedicarboxylic acid, which are all encompassed by the term “itaconate”. Itaconate has been assigned Chemical Abstracts Service registry number (CAS No.) 97-65-4.

Typically, a derivative or analogue of itaconate, or a pharmaceutically acceptable salt thereof is one that exhibits similar functional properties to itaconate. Preferably, a derivative or analogue of itaconate, or a pharmaceutically acceptable salt thereof inhibits SDH. A derivative or analogue of itaconate, or a pharmaceutically acceptable salt thereof, may exhibit improved SDH inhibitory activity when compared to itaconate, or may exhibit at least 50% (e.g. at least 60%, 70%, 80% or 90%) of the SDH inhibitory activity of itaconate.

References herein to itaconate include, derivatives, analogues, hydrates, solvates, prodrug and salt forms as described herein unless explicitly states to the contrary.

### Aptamers

Aptamers are generally nucleic acid molecules that bind a specific target molecule. Aptamers can be engineered completely in vitro, are readily produced by chemical synthesis, possess desirable storage properties, and elicit little or no immunogenicity in therapeutic applications. These characteristics make them particularly useful in pharmaceutical and therapeutic utilities.

As used herein, “aptamer” refers in general to a single or double stranded oligonucleotide or a mixture of such oligonucleotides, wherein the oligonucleotide or mixture is capable of binding specifically to a target. Oligonucleotide aptamers will be discussed here, but the skilled reader will appreciate that other aptamers having equivalent binding characteristics can also be used, such as peptide aptamers.

In general, aptamers may comprise oligonucleotides that are at least 5, at least 10 or at least 15 nucleotides in length. Aptamers may comprise sequences that are up to 40, up to 60 or up to 100 or more nucleotides in length. For example, aptamers may be from 5 to 100 nucleotides, from 10 to 40 nucleotides, or from 15 to 40 nucleotides in length. Where possible, aptamers of shorter length are preferred as these will often lead to less interference by other molecules or materials.

Aptamers may be generated using routine methods such as the Systematic Evolution of Ligands by Exponential enrichment (SELEX) procedure. SELEX is a method for the in vitro evolution of nucleic acid molecules with highly specific binding to target molecules. It is described in, for example, U.S. Pat. Nos. 5,654,151, 5,503,978, 5,567,588 and WO 96/38579.

The SELEX method involves the selection of nucleic acid aptamers and in particular single stranded nucleic acids capable of binding to a desired target, from a collection of oligonucleotides. A collection of single- stranded nucleic acids (e.g., DNA, RNA, or variants thereof) is contacted with a target, under conditions favourable for binding, those nucleic acids which are bound to targets in the mixture are separated from those which do not bind, the nucleic acid-target complexes are dissociated, those nucleic acids which had bound to the target are amplified to yield a collection or library which is enriched in nucleic acids having the desired binding activity, and then this series of steps is repeated as necessary to produce a library of nucleic acids (aptamers) having specific binding affinity for the relevant target.

### Peptides and Peptidomimetics

In addition, the invention encompasses the use of peptide and peptidomimetic agents to inhibit SDH. For example, the use of peptides, stapled peptides, peptoids and peptidomimetics that would directly or indirectly inhibit SDH is embraced by the present invention.

Peptidomimetics are compounds which mimic a natural peptide or protein with the ability to interact with the biological target and produce the same biological effect. Peptidomimetics may have advantages over peptides in terms of stability and bioavailability associated with a natural peptide. Peptidomimetics can have main- or side-chain modifications of the parent peptide designed for biological function. Examples of classes of peptidomimetics include, but are not limited to, peptoids and β-peptides, as well as peptides incorporating D-amino acids.

Methods for producing synthetic peptides and peptidomimetics (such as peptoids) are known in the art, as are the sequences of SDH and its ligands. Thus, it would be routine for one of skill in the art to produce suitable synthetic peptides and peptidomimetics which directly or indirectly inhibit SDH using known techniques and based on the known SDH and SDH ligand sequences.

### Antibodies and Antigen-Binding Fragments Thereof

An agent which inhibits SDH may be an antibody, or an antigen binding fragment thereof as defined herein. An antibody according to the invention may be polyclonal or monoclonal, preferably monoclonal.

An antibody of the invention and antigen-binding fragments thereof may be derived from any species by recombinant means. For example, the antibodies or antigen-binding fragments may be mouse, rat, goat, horse, swine, bovine, chicken, rabbit, camelid, donkey, human, or chimeric versions thereof. For use in administration to humans, non-human derived antibodies or antigen-binding fragments may be genetically or structurally altered to be less antigenic upon administration to the human patient.

Especially preferred are human or humanized antibodies, especially as recombinant human or humanized antibodies as defined herein.

An antibody of the invention and antigen-binding fragments thereof disclosed herein can be further modified using conventional techniques known in the art, for example, by using amino acid deletion(s), insertion(s), substitution(s), addition(s), and/or recombination(s) and/or any other modification(s) known in the art, either alone or in combination. Methods for introducing such modifications in the DNA sequence underlying the amino acid sequence of an immunoglobulin chain arc well known to the person skilled in the art.

The antibodies of the invention or antigen-binding fragments thereof may have any antibody format. In some embodiments, the antibody has the “conventional” format described above. Alternatively, the antibody can be in some embodiments a Fab fragment. The antibody according to the invention can also be a Fab′, an Fv, an scFv, an Fd, a V NAR domain, an IgNAR, an intrabody, an IgG CH2, a minibody, a single-domain antibody, an Fcab, an scFv-Fc, F(ab′)2, a di-scFv, a bi-specific T-cell engager (BiTE®), a F(ab′)3, a tetrabody, a triabody, a diabody, a DVD-Ig, an (scFv)2, or a mAb2.

### Nucleic Acids

The agents which inhibit SDH according to the invention may be nucleic acids as defined herein.

A nucleic acid agent of the invention may inhibit SDH expression. Such nucleic acids include “antisense nucleic acids”, by which is meant an RNA or DNA molecule that binds to another RNA or DNA (target RNA, DNA), whether an SDH RNA or DNA as defined herein (e.g. in the case of direct SDH inhibition), or a non-SDH RNA or DNA (e.g. in the case of indirect inhibition). Non-limiting examples of antisense nucleic acids include, for example, antisense RNA or DNA molecules, interference RNA (RNAi), micro RNA, decoy RNA molecules, siRNA, enzymatic RNA, therapeutic editing RNA and agonist and antagonist RNA, antisense oligomeric compounds, antisense oligonucleotides, external guide sequence (EGS) oligonucleotides, alternate splicers, primers, probes, and other oligomeric compounds that hybridize to at least a portion of the target nucleic acid (such as the SDHA gene). As such, these nucleic acids may be introduced in the form of single-stranded, double- stranded, partially single-stranded, or circular oligomeric compounds.

A nucleic acid agent of the invention may increase ACOD1 expression. By way of non-limiting example, a nucleic acid of the invention may comprise a nucleic acid sequence encoding for ACOD1 as defined herein. Typically, the nucleic acid sequence encoding for ACOD1 is operably linked to a promoter capable to expressing said nucleic acid sequence. Examples of inducible and non-inducible promoters are known in the art. Thus, it would be routine for one of skill in the art to select a suitable promoter.

A nucleic acid of the invention may increase ACOD1 expression through modulation of an upstream transcriptional program. By way of non-limiting example, a nucleic acid of the invention may increase ACOD1 expression through modulation of a ZBP1- and/or RIPK-dependent transcriptional pathways. The modulation may be to up-regulate a ZBP1- and/or RIPK-dependent transcriptional pathway.

### Vectors and Plasmids

The present invention provides a vector that expresses an agent of the invention. In other words, an agent of the invention may be provided by means of a vector.

The vector may be a viral vector. Such a viral vector may be an adenovirus (of a human serotype such as AdHu5, a simian serotype such as ChAd63, ChAdOXI or ChAdOX2, or another form), an adeno-associated viral (AAV) vector or poxvirus vector (such as a modified vaccinia Ankara (MVA)). ChAdOXI and ChAdOX2 are disclosed in WO2012/172277. ChAdOX2 is a BAC -derived and E4 modified AdC68-based viral vector.

Viral vectors are usually non-replicating or replication impaired vectors, which means that the viral vector cannot replicate to any significant extent in normal cells (e.g. normal human cells), as measured by conventional means—e.g. via measuring DNA synthesis and/or viral titre. Non-replicating or replication impaired vectors may have become so naturally (i.e. they have been isolated as such from nature) or artificially (e.g. by breeding in vitro or by genetic manipulation). There will generally be at least one cell-type in which the replication-impaired viral vector can be grown—for example, modified vaccinia Ankara (MVA) can be grown in CEF cells. In one embodiment, the vector is selected from a human or simian adenovirus, AAV or a poxvirus vector.

Typically, the viral vector is incapable of causing a significant infection in an animal individual, typically in a mammalian individual such as a human or other primate.

The invention further provides a DNA vector that expresses an agent of the invention, such as a plasmid-based DNA vaccine. In one embodiment the DNA vector is capable of expression in a mammalian cell expression system, such as an immunised cell.

The vector may be an RNA vector, such as a self-amplifying RNA vaccine (Geall, A. J. et al., Proc Natl Acad Sci USA 2012; 109(36) pp. 14604-9).

The present invention also provides virus-like particles (VLP) and/or fusion proteins comprising an agent of the invention, as described herein. References herein to vectors of the invention may apply equally to VLP and/or fusion proteins of the invention.

### Drug Delivery Systems

An agent, vector or composition of the invention may be delivered by means of a drug delivery system. Drug delivery systems may be used to increase delivery of an agent, vector or composition of the invention; increase uptake of an agent, vector or composition of the invention by a target cell or tissue; and/or to increase the efficacy of an agent, vector or composition of the invention.

Any appropriate drug delivery system may be used to deliver an agent, vector or composition of the invention. Conventional drug delivery systems are known in the art. By way of non-limiting example, appropriate drug delivery systems include liposomes, immunoliposomes, nanoparticles and conjugates. Thus, it would be routine for one of skill in the art to select a suitable drug delivery system. Liposome drug delivery systems are referred to interchangeably herein as liposome-based drug delivery systems.

The skilled person would understand that the choice of drug delivery system may depend on the particular indication and/or tissue to be treated.

As discussed herein, phagocytic cells, such as different populations of macrophages are particularly relevant in the context of fibrosis. Therefore, drug delivery systems (e.g. liposomes or nanoparticles) specifically adapted for phagocytic cells may be used according to the invention. For example, drug delivery systems which specifically or preferentially target phagocytic cells may be used according to the invention. By way of non-limiting example phosphatidyl choline: cholesterol liposomes are a preferred drug delivery system of the invention. Any suitable ratio of phosphatidyl choline: cholesterol may be used in a liposome of the invention, however, liposomes with a 70:30 molar ratio percentage of phosphatidyl choline: cholesterol are particularly preferred. Such liposome drug delivery systems may further be conjugated to antibodies, or antigen binding fragments thereof, which target phagocytic cell-specific cell surface markers. Liposome drug delivery systems may also be glycosyslated, preferably, mannosylated. Drug delivery systems (e.g. liposomes or nanoparticles) may be suited for delivery to phagocytic cells based on their size distribution and/or surface charge, preferably both. Typically, the drug delivery systems will have an average size of between 1 to 5 μm, preferably 1.5 to 2 μm.

### Therapeutic Indications

The agents, vectors, compositions and drug delivery systems as described herein are useful in the treatment of tissue fibrosis.

Fibrosis is a pathological mechanism which occurs in numerous organs and diseases. Fibrosis results from abnormal tissue repair and is associated with persistent and/or severe tissue damage and cellular stress. Failure to adequately contain or eliminate factors triggering fibrosis can exacerbate inflammation and chronic wound-healing responses, resulting in continued tissue damage and inadequate regeneration and, ultimately, fibrosis.

Although fibrosis and inflammation can occur simultaneously, the mechanisms underlying the two processes are distinct. For example, in idiopathic pulmonary fibrosis (IPF) inflammation is often mild and patchy, and clinical trials using anti-inflammatory drugs to treat IPC failed to treat outcomes and instead have been found to increase mortality.

Although differing in aetiology and causative mechanisms, fibrotic diseases all have abnormal and exaggerated accumulation of extracellular matrix (ECM) components, mainly fibrillar collagens. The resulting fibrosis disturbs the normal architecture of affected organs, which ultimately leads to their dysfunction and failure. Due to the common mechanism underlying fibrosis in numerous tissues and diseases, the agents, compositions and drug delivery systems of the invention are useful in treating tissue fibrosis in a range of diseases and tissue types.

Accordingly, the present invention relates to the treatment of tissue fibrosis and diseases and disorders associated with tissue fibrosis. Non-limiting examples of tissue fibrosis include pulmonary fibrosis, liver fibrosis, kidney fibrosis, intestinal fibrosis, cardiac fibrosis, myelofibrosis and/or skin fibrosis. Preferably the invention relates to the treatment of pulmonary (lung) fibrosis. Non-limiting examples of diseases and disorders associated with tissue fibrosis include chronic fibrosing interstitial lung disease, connective tissue diseases (for example, scleroderma), and hepatic cirrhosis. Preferably the invention relates to the treatment of chronic fibrosing interstitial lung disease, even more preferably to the treatment of IPF.

### Monocyte-Recruited Macrophages

As shown in the Examples herein, the inventors have demonstrated that monocyte derived macrophages are recruited to sites of tissue fibrosis, and that these monocyte-recruited macrophages (Mo-Ms) are less metabolically active (quiescent) than tissue-resident macrophages (Tr-Ms), which are highly oxidative. The inventors have surprisingly shown that Mo-Ms highly express ACOD1, and so produce high levels of itaconate, facilitating SDH inhibition. Recruitment of Mo-Ms, e.g. monocyte-recruited airway macrophages (Mo-AMs) in the case of pulmonary fibrosis, therefore delivers itaconate to the site of tissue fibrosis, which then inhibits SDH an provides one or more treatment outcome as described herein, including changing the metabolic and/or fibrotic phenotype of Tr-Ms (such as Tr-AMs). Furthermore, the inventors have unexpectedly shown that transfer of Mo-Ms into the lungs of a murine model of IPF rescues the fibrotic phenotype. The inventors have also shown that culture of AMs from IPF patients with itaconate inhibits pro-fibrotic and pro-inflammatory pathways. Therefore, inhibition of SDH according to the invention, whether by delivery of an agent, or a vector, composition or drug delivery system comprising said agent, or by delivery of a Mo-M population has therapeutic potential for tissue fibrosis.

Accordingly, the invention also provides a population of Mo-Ms for use in the treatment or prevent of tissue fibrosis.

Mo-Ms are macrophages which originate from monocytes circulating in the blood. One of ordinary skill in the art would readily be able to differentiate between Mo-Ms and Tr-Ms. By way of non-limiting example, Mo-Ms can be distinguished from Tr-Ms by their marker expression profile. For example, Mo-Ms express (i.e. are positive for) one or more of CD14, CD163, CD206, HLA-Dr, CD71, fatty acid binding protein-4 (FABP4) and Ficolin-1. Therefore, one or more of these markers, or a combination thereof, may be used to identify Mo-Ms and hence distinguish between Mo-Ms and Tr-Ms. A marker expression profile of CD14, CD163, CD206, HLA-Dr, CD71, FABP4 and Ficolin-1 may be used to identify Mo-Ms.

Mo-Ms for use according to the present invention typically express ACOD1. This is in contrast to Tr-Ms within a tissue where fibrosis is occurring, which typically do not express ACOD1, or express ACOD1 at low levels. Typically, Mo-Ms have an ACOD1 expression level at least 0.25 fold, at least 0.5 fold, at least 0.75 fold, at least 1 fold, at least 1.5 fold, or at least 2 fold higher than the ACOD1 expression of Tr-Ms. Preferably, Mo-Ms have an ACOD1 expression level at least 1.5 fold higher than the ACOD1 expression of Tr-Ms.

Mo-Ms for use according to the present invention typically have low metabolic activity, or are quiescent. This may be described interchangeably herein as having a quiescent metabolic phenotype. This is in contrast to Tr-Ms within a tissue where fibrosis is occurring, are typically highly metabolically active. High levels of metabolic activity are associated with high oxygen consumption rates (OCR), whereas low levels of metabolic activity (quiescence) is associated with low OCR. Metabolic activity may also be defined in terms of a cells extracellular acidification rate (SCAR), glucose consumption, GLUT (glucose transporter) expression, isocitrate dehydrogenase (IDH2) expression, SDH expression and/or malate dehydrogenase (MDH) expression

The Mo-Ms for use according to the present invention may be autologous (i.e. derived from the subject to be treated), or allogenic (i.e. from another individual of the same species).

The Mo-Ms may be administered directly to an individual to be treated. Typically, direct administration is by intravenous infusion. Typically, about 1×10⁶ to about 5×10⁹ Mo-Ms are delivered per administration, for example, about 1×10⁶, 5×10⁶, 1×10⁷, 5×10⁷, 1×10⁸, 5×10⁸, 1×10⁹, or 5×10⁹ Mo-Ms per administration.

Alternatively, the population of Mo-Ms may be recruited following administration of an agent, compound or composition which stimulates targeting of Mo-Ms to a tissue to be treated. Typically, the Mo-Ms are recruited from the individual's own circulating Mo-Ms. Agents capable of recruiting circulating Mo-Ms are known to those skilled in the art. Typically, a cytokine is used to recruit circulating Mo-Ms. By way of non-limiting example, recombinant chemokine ligand 2 (CCL2; also known as monocyte chemoattractant protein 1) may be used to recruit circulating Mo-Ms.

### Treatment Outcomes

“Treatment” according to the present invention may be defined as providing a treatment outcome as defined below. These definitions may apply to therapeutic and prophylactic treatments as described herein.

Treatment may modify the metabolic and/or fibrotic phenotype of Tr-Ms. In particular, “treatment” may be defined as increasing the metabolic phenotype and/or decreasing the fibrotic phenotype of Tr-Ms. An increase in the metabolic phenotype of Tr-Ms may be defined as an increase in the proportion of CD11⁺/MHCII⁺ Tr-Ms and/or a decrease in the proportion of CD11b⁻/MHCII⁻ Tr-Ms. For example, treatment may: (i) increase the proportion of CD11b+/M HCII⁺ Tr-Ms by at least 10%, at least 15%, at least 20% or more; and/or (ii) decrease the proportion of CD11b⁻/MHCII⁻ Tr-Ms by at least 2%, at least 3%, at least 4%, at least 5% or more.

Alternatively or in addition, treatment may modify the metabolic and/or fibrotic phenotype of fibroblasts within the tissue to be treated. In particular, “treatment” may be defined as decreasing the metabolic and/or fibrotic phenotype of fibroblasts within the tissue to be treated. A decrease in the metabolic phenotype of fibroblasts may be defined as a decrease in the OCR, maximal respiration rate and/or spare respirator capacity of the fibroblasts. A decrease in the fibrotic phenotype of fibroblasts may be defined as a decrease in the fibroblast proliferation rate and/or a decrease in the wound healing capacity of the fibroblasts. A decrease in the fibrotic phenotype of fibroblasts may also be defined in terms of the expression of fibrotic markers. By way of non-limiting example, a decrease in the fibrotic phenotype of the fibroblasts may be defined as a decrease in matrix metalloproteinase, TGFβ1 and/or CD71 expression.

Treatment may modify the metabolic and/or fibrotic phenotype of Tr-Ms and fibroblasts. In particular, “treatment” may be defined as: (i) increasing the metabolic phenotype of Tr-Ms; (ii) decreasing the fibrotic phenotype of Tr-Ms; (iii) decreasing the metabolic phenotype of fibroblasts; and or (iv) decreasing the fibrotic phenotype of fibroblasts; within the tissue to be treated. Any combination of (i)-(iv) is encompassed by the present invention. Preferably treatment encompasses all of (i)-(iv).

Treatment according to the invention may result in: (a) an improvement in the fibrosis of the tissue; (b) a decrease in tissue collagen expression, preferably Col3α1, Col1α1 and/or Col4α1; (c) a decrease in tissue fibronectin (Fn1) expression; (d) a decrease in IL-1β expression in fibroblasts obtained from the tissue; and/or (e) a decrease in hydroxyproline levels. Any combination of (a)-(e) is encompassed by the present invention. Preferably treatment encompasses all of (a)-(e).

By way of non-limiting example, when the tissue fibrosis to be treated according to the invention is pulmonary fibrosis, treatment may result in: (a) an improvement in lung function; (b) a reduction in the decline of forced vital capacity; (c) preservation or improvement of exercise capacity; (d) a reduction in the progression of fibrosis as quantified by high resolution computed tomography; (e) preservation or improvement of quality of life; and/or (f) an improvement in survival. Any combination of (a)-(f) is encompassed by the present invention. Preferably treatment encompasses all of (a)-(f).

An improvement in lung function may be defined as one or more of (i) an increase in force vital capacity (FVC); (ii) an increase in total lung capacity; and/or (iii) and increase in the transfer capacity of the lung for the uptake of carbon monoxide, as measured by a gas transfer (TLco) test; or (iv) any combination thereof. Any combination of (i)-(iii) is encompassed by the present invention. Preferably treatment encompasses all of (i)-(iii).

Treatment according to the present invention may result in any combination of the treatment outcomes as described herein.

### Therapy

The invention provides an agent (as described herein), vectors and compositions comprising said agent, drug delivery systems for delivering said agent and Mo-M populations for use in the treatment or prevention of tissue fibrosis. Said agent inhibits SDH, and this inhibition may be direct or indirect. Preferably agents which indirectly inhibit SDH do so by increasing the expression and/or activity of ACOD1 as described herein.

The term “treat” or “treating” as used herein encompasses prophylactic treatment (e.g. to prevent onset of tissue fibrosis) as well as corrective treatment (treatment of an individual already suffering from tissue fibrosis). Preferably, the term “treat” or “treating” as used herein means corrective treatment. The term “treat” or “treating” encompasses treating both tissue fibrosis, symptoms thereof and diseases/disorder associated therewith. In some embodiments the term “treat” or “treating” refers to a symptom of tissue fibrosis.

The “treatment” may be defined as providing a treatment outcome as defined herein. For example, the “treatment” may modify: (i) the metabolic and/or fibrotic phenotype of Tr-Ms; and/or (ii) the metabolic and/or fibrotic phenotype of fibroblasts within the tissue to be treated; as described herein. In particular, “treatment” may be defined as: (i) increasing the metabolic phenotype and/or decreasing the fibrotic phenotype of Tr-Ms; and/or (ii) decreasing the metabolic and/or fibrotic phenotype of fibroblasts within the tissue to be treated; as described herein.

An agent, vector, composition, drug delivery system or Mo-M population of the invention may be used in the treatment of an individual having Tr-Ms with reduced ACOD1 expression. An individual may be screened for the ACOD1 expression of their Tr-Ms prior to treatment (e.g. using a sample or biopsy of the tissue to be treated), and may be selected for treatment based on the level of expression of the Tr-Ms. In the case of an individual to be treated for pulmonary fibrosis, the tissue sample used to test for Tr-M ACOD1 expression levels may be a bronchoalveolar lavage (BAL) sample. Typically, the level of ACOD1 expression in Tr-Ms comprised in sample (e.g. a BAL sample) obtained from an individual to be treated is reduced compared to the level in a control sample. The control sample may be from an individual that does not have tissue fibrosis (e.g. if BAL samples are used, pulmonary fibrosis).

An agent, vector, composition, drug delivery system or Mo-M population of the invention may be used in the treatment of an individual having reduced levels of itaconate within the tissue undergoing fibrosis. An individual may be screened for the level of itaconate in the tissue to be treated prior to treatment (e.g. using a sample or biopsy of the tissue to be treated), and may be selected for treatment based on the level of itaconate in the tissue. In the case of an individual to be treated for pulmonary fibrosis, the tissue sample used to test for the level may be a BAL sample. Typically, the level of itaconate in a BAL sample obtained from an individual to be treated is reduced compared to the level in a control BAL sample (e.g. from an individual that does not have pulmonary fibrosis).

A “therapeutically effective amount” is any amount of an agent, vector, composition, drug delivery system or Mo-M population of the invention which, when administered alone or in combination to a patient for treating tissue fibrosis (or preventing further tissue fibrosis) or a symptom thereof or a disease associated therewith is sufficient to provide such treatment of the tissue fibrosis, or symptom thereof, or associated disease. A “prophylactically effective amount” is any amount of an agent, vector, composition, drug delivery system or Mo-M population of the invention that, when administered alone or in combination to an individual inhibits or delays the onset or reoccurrence of tissue fibrosis, or a symptom thereof or disease associated therewith). In some embodiments, the prophylactically effective amount prevents the onset or reoccurrence of tissue fibrosis entirely. “Inhibiting” the onset means either lessening the likelihood of tissue fibrosis onset (or symptom thereof or disease associated therewith) or preventing the onset entirely.

The terms “subject”, “individual” and “patient” are used interchangeably herein to refer to a mammalian individual. Generally, the individual may be human; in other words, in one embodiment, the “individual” is a human. The individual may not have been previously diagnosed as having tissue fibrosis (or symptom thereof or disease associated therewith). Alternatively, the individual may have been previously diagnosed as having tissue fibrosis (or symptom thereof or disease associated therewith). The individual may also be one who exhibits disease risk factors, or one who is asymptomatic for tissue fibrosis (or symptom thereof or disease associated therewith). The individual may also be one who is suffering from or is at risk of developing tissue fibrosis (or symptom thereof or disease associated therewith).

Administration of an agent of the invention (e.g. itaconate), vector, composition, drug delivery system or Mo-M population of the invention may be by any appropriate route. Non-limiting examples of conventional routes include inhalation; intraperitoneal, intravenous, intra-arterial, subcutaneous, and/or intramuscular injection; infusion; rectal, vaginal, topical and oral administration. The most appropriate administration route may be selected on the site of the fibrosis to be treated and/or prevented. By way of example, where the fibrosis to be treated is pulmonary fibrosis, such as IPF, the agent (e.g. itaconate), vector, composition, drug delivery system or Mo-M population of the invention may be administered by inhalation. In some preferred embodiments, the agent (e.g. itaconate), vector, composition, drug delivery system or Mo-M population of the invention is administered by inhalation, preferably oropharyngeal inhalation and/or nasal inhalation. In a particularly preferred embodiment, itaconate is administered by oropharyngeal inhalation.

It will be appreciated by one of skill in the art that the appropriate dosage of an agent (e.g. itaconate), vector, composition, drug delivery system or Mo-M population of the invention, can vary from individual to individual. Determining the optimal dosage will generally involve the balancing of the level of therapeutic benefit against any risk or deleterious side effects. The selected dosage level will depend on a variety of factors including, the route of administration, the severity of the individual's/patient's fibrosis, and the species, sex, age, weight, condition, general health, and prior medical history of the individual/patient. Advantageously, the present inventors have identified suitable dosages of an agent of the invention, particularly itaconate, which provide the anti-fibrotic effects claimed. Typically, the agent, particularly itaconate, is administered at a dose of about 0.1 to 20 mg/kg. Preferably, the agent, particularly itaconate, is administered at a dose of about 0.1 to 10 mg/kg, even more preferably at dose of about 5 to 10 mg/kg. In a particularly preferred embodiment, itaconate is administered at a dose of about 5 to 10 mg/kg by oropharyngeal administration.

The frequency of dosing selected may also be dependent on a range of factors. The skilled person will be able to select the most suitable dosing regimen appropriate for the individual. Typically, an agent (e.g. itaconate), vector, composition, drug delivery system or Mo-M population is administered between about once every three months to about four times per day. For example, the agent (e.g. itaconate), vector, composition or drug delivery system may be administered once every three months, once per month, twice per month, once per week, twice per week, 3 times per week, 4 times per week, 5 times per week, 6 times per week, once a day, twice a day, 3 times per day, 4 times per day or more. Preferably, the agent (e.g. itaconate), vector, composition or drug delivery system is administered about once per day.

An agent, vector, composition, drug delivery system or Mo-M population of the invention may have a treatment outcome as defined herein within 8-52 weeks (preferably within 36 weeks, more preferably within 24 weeks, even more preferably within 12 weeks) from baseline. Preferably, administration of the agent, vector, composition, drug delivery system or Mo-M population of the invention may provide a treatment outcome within 36 weeks, more preferably within 24 weeks, even more preferably within 12 weeks.

The treatment outcome may be sustained (e.g. maintained) subsequent to and/or during treatment for several weeks or months or years. An agent, vector, composition, drug delivery system or Mo-M population of the invention may provide a sustained treatment outcome for at least 5, 10, 12, 16, 18, 20, 22, 24, 38, 32, 36, 40, 52, 78 or 104 weeks. For example, administration of an agent, vector, composition, drug delivery system or Mo-M population of the invention may provide a sustained treatment outcome for at least 5 weeks, at least 10 weeks, at least 20 weeks, or at least 52 weeks.

An agent, vector, composition, drug delivery system or Mo-M population of the invention may be used in combination with one or more additional active ingredient or therapeutic, such as another anti-fibrotic agent and/or an anti-inflammatory. By way of non-limiting example, the agent, vector, composition, drug delivery system or Mo-M population of the invention may be used in combination with pirfenidone and/or nintedanib. Other suitable anti-fibrotic agents which may be used in combination with an agent, vector, composition, drug delivery system or Mo-M population of the invention include pamrevlumab (an anti-connective tissue growth factor monoclonal antibody), ziritaxestat (also known as GLPG 1690, an autotaxin inhibitor), PRM-151 (a pentraxin-2 recombinant protein and GB0139 (a galectin 3 inhibitor).

The one or more additional active ingredient or therapeutic may be administered sequentially (before or after) the agent, vector, composition, drug delivery system or Mo-M population of the invention. The one or more additional active ingredient or therapeutic may be administered simultaneously with the agent, vector, composition, drug delivery system or Mo-M population of the invention.

The invention also provides method for the treatment or prevention of fibrosis comprising administering an agent, vector, composition, drug delivery system or Mo-M population of the invention which inhibits SDH.

The invention also provides an agent, vector, composition, drug delivery system or Mo-M population of the invention which inhibits SDH for use in the manufacture of a medicament for the treatment or prevention of fibrosis.

### Compositions and Formulations

The invention provides compositions, particularly pharmaceutical compositions, comprising an agent, vector, composition, drug delivery system or Mo-M population of the invention and a pharmaceutically acceptable excipient, diluent, adjuvant, immunoregulatory agent and/or antimicrobial compound.

The agent may be in the form of a pro-drug, analogue, derivate, salt, hydrate or solvate as described herein.

Compositions or formulations comprising an agent, vector, composition, drug delivery system or Mo-M population of the invention may further comprise one or more additional active ingredient or therapeutic, such as another anti-fibrotic agent and/or an anti-inflammatory as described herein. The agent, vector, composition, drug delivery system or Mo-M population of the invention and the one or more additional active ingredient or therapeutic may be provided as a kit of parts.

As described herein, administration of immunogenic compositions, therapeutic formulations, medicaments and prophylactic formulations is generally by conventional routes, with inhalation and particularly oropharyngeal inhalation, being preferred.

Formulation of an agent, vector, composition, drug delivery system or Mo-M population of the invention may therefore be adapted using routine practice to suit the preferred route of administration.

Formulations suitable for distribution as aerosols are preferred, and it would be routine for one of ordinary skill in the art to prepare such formulations.

By way of further non-limiting example, an agent, vector, composition, drug delivery system or Mo-M population of the invention, compositions or therapeutic/prophylactic formulations and/or medicaments thereof may be prepared as injectables, either as liquid solutions or suspensions. Solid forms suitable for solution in, or suspension in, liquid prior to injection may alternatively be prepared. The preparation may also be emulsified, or the peptide encapsulated in liposomes or microcapsules. The agent, vector, composition or drug delivery system of the invention may also be formulated as a dry-powder formulation.

The active immunogenic ingredients are often mixed with excipients which are pharmaceutically acceptable and compatible with the active ingredient. Suitable excipients are, for example, water, saline, dextrose, glycerol, ethanol, or the like and combinations thereof. In addition, if desired, the composition may contain minor amounts of auxiliary substances such as wetting or emulsifying agents, pH buffering agents, and/or adjuvants which enhance the effectiveness of the vaccine.

Generally, the carrier is a pharmaceutically-acceptable carrier. Non-limiting examples of pharmaceutically acceptable carriers include water, saline, and phosphate-buffered saline. In some embodiments, however, the composition is in lyophilized form, in which case it may include a stabilizer, such as BSA. In some embodiments, it may be desirable to formulate the composition with a preservative, such as thiomersal or sodium azide, to facilitate long term storage.

Examples of additional adjuvants which may be effective include but are not limited to: complete Freunds adjuvant (CFA), Incomplete Freunds adjuvant (IFA), Saponin, a purified extract fraction of Saponin such as Quil A, a derivative of Saponin such as QS-21, lipid particles based on Saponin such as ISCOM/ISCOMATRIX, E. coli heat labile toxin (LT) mutants such as LTK63 and/ or LTK72, aluminium hydroxide, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(I′-2′-dipalmitoyl-sn-glycero-3-hydroxyphosphoryl oxy)-ethylamine (CGP 19835 A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion, the MF59 formulation developed by Novartis, and the AS02, AS01, AS03 and AS04 adjuvant formulations developed by GSK Biologicals (Rixensart, Belgium).

Examples of buffering agents include, but are not limited to, sodium succinate (pH 6.5), and phosphate buffered saline (PBS; pH 6.5 and 7.5).

Additional formulations which are suitable for other modes of administration include suppositories and, in some cases, oral formulations. For suppositories, traditional binders and carriers may include, for example, polyalkylene glycols or triglycerides; such suppositories may be formed from mixtures containing the active ingredient in the range of 0.5% to 10%, preferably 1%-2%.

Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders.

### Screening Assays

The present inventors are the first to demonstrate that treating tissue fibrosis by inhibiting SDH results in quantifiable phenotypic changes in cells, particularly Tr-Ms and fibroblasts, within the tissue. Therefore, quantifying these changes in an in vitro setting has the potential to identify other agents which inhibit the progression of fibrosis, and hence have therapeutic potential for the treatment of tissue fibrosis.

Accordingly, the invention also provides a method for identifying an agent which inhibits fibrosis progression comprising the steps of: (a) culturing cells in vitro; (b) adding a test agent to the cultured cells; and (c) determining a change in the metabolic phenotype of the cells in response to the test agent. The change in metabolic phenotype of the cells is a reduction or increase in the metabolic phenotype of the cells, as defined herein.

Any type of test agent may be employed in a method of the invention. The skilled person will be familiar with the various types of test agents which may be added to cultured cells in vitro. The test agent may be any type of agent as described herein. By way of non-limiting example, the test agent may be a small molecule, a nucleic acid agent (for example, an siRNA, a plasmid, an antisense oligonucleotide or a nucleic acid aptamer), an antibody or antibody-fragment thereof, or a peptide aptamer. Vectors, compositions or drug delivery systems comprising or expressing a test agent may also be employed in a screening method of the invention. Any disclosure herein in relation to vectors, drug delivery systems and compositions applies equally and without limitation for vectors, drug delivery systems and compositions comprising or expressing test agents for use in a screening method of the invention. By way of non-limiting example, a vector may be an adeno-associated viral vector, an adenoviral vector or a lentiviral vector comprising a nucleic sequence encoding the test agent. By way of a further non-limiting example, a liposomal drug delivery system comprising a test agent may be used.

Any cell type capable of being cultured in vitro may be utilised in a screening method of the invention. Typically, the cells are primary cells (i.e. cells derived from animal tissues) or cell lines. The cells used in a method of the invention may be a cell type involved in fibrosis. By way of non-limiting example, the cells may be a stromal cell or immune cell. In view of their role in fibrosis, fibroblasts and macrophages are particularly useful in methods of the invention. Thus, the cells used may be fibroblasts or macrophages, for example, Tr-Ms or Mo-Ms. Preferably fibroblasts and/or Tr-Ms are used.

The cells may be derived from a tissue which can develop fibrosis. By way of non-limiting example, the cells may be derived from lung tissue, or from a BAL sample. The cells may be derived from an individual to be treated, i.e. from an individual with fibrosis already occurring in a tissue to be treated. The cells may be derived from a biopsy sample of an individual with fibrosis. Cells, particularly Tr-Ms and/or Mo-Ms may be isolated from peripheral blood using flow cytometry.

The metabolic phenotype assessed using a screening method of the invention may be a metabolic phenotype as described herein in the context of treatment outcomes. By way of non-limiting example, a decrease in the metabolic phenotype may be defined as a decrease in the OCR, maximal respiration rate and/or spare respirator capacity of the cells (e.g. fibroblasts). By way of non-limiting example, an increase in the metabolic phenotype may be defined as an increase in the OCR, maximal respiration rate and/or spare respirator capacity of the cells (e.g. Tr-Ms). An increase or decrease in metabolic phenotype may be an increase or decrease of SCAR, glucose consumption, GLUT expression, isocitrate dehydrogenase (IDH2) expression, SDH expression and/or malate dehydrogenase (MDH) expression.

The change in metabolic phenotype may be compared with a control. Any appropriate control may be used, and it is within the standard competency of one of ordinary skill in the art to select an appropriate control. Examples of suitable controls are described herein. For example, a control may be a population of the same cell type (preferably from the same source), wherein the control cells are cultured in the same conditions as the cells exposed to the test agent, vector, composition or drug delivery system, but wherein the control cells are not exposed to the test agent, vector, composition or drug delivery system.

A screening method of the invention may consist of the steps described herein (carried out in sequentially in the described order), or may comprise additional steps. By way of non-limiting example, the method may further comprise a step of determining a change, particularly a reduction, in the fibrotic phenotype of the cells in response to the test agent, vector, composition or drug delivery system. The fibrotic phenotype assessed using a screening method of the invention may be a fibrotic phenotype as described herein in the context of treatment outcomes. Further non-limiting example of additional steps include isolating and/or the cells after exposure to the test agent, vector, composition or drug delivery system.

### SEQUENCE HOMOLOGY

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position- Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264(4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501 -509 (1992); Gibbs sampling, see, e.g., C. E. Lawrence et al., Detecting Subtle Sequence Signals: A Gibbs Sampling Strategy for Multiple Alignment, 262(5131) Science 208-214 (1993); Align-M, see, e.g., Ivo Van Walle et al., Align-M—A New Algorithm for Multiple Alignment of Highly Divergent Sequences, 20(9) Bioinformatics:1428-1435 (2004).

Thus, percent sequence identity is determined by conventional methods. See, for example, Altschul et al., Bull. Math. Bio. 48: 603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes).

The “percent sequence identity” between two or more nucleic acid or amino acid sequences is a function of the number of identical positions shared by the sequences. Thus, % identity may be calculated as the number of identical nucleotides/amino acids divided by the total number of nucleotides/amino acids, multiplied by 100. Calculations of % sequence identity may also take into account the number of gaps, and the length of each gap that needs to be introduced to optimize alignment of two or more sequences. Sequence comparisons and the determination of percent identity between two or more sequences can be carried out using specific mathematical algorithms, such as BLAST, which will be familiar to a skilled person.

### ALIGNMENT SCORES FOR DETERMINING SEQUENCE IDENTITY

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

The percent identity is then calculated as:

$$\frac{Total number of identical matches}{\begin{matrix}[ length of the longer sequence plus the \\ number of gaps introduced into the longer \\ sequence in order to align the two sequences ]\end{matrix}} \times 100$$

Substantially homologous polypeptides are characterized as having one or more amino acid substitutions, deletions or additions. These changes are preferably of a minor nature, that is conservative amino acid substitutions (as described herein) and other substitutions that do not significantly affect the folding or activity of the polypeptide; small deletions, typically of one to about 30 amino acids; and small amino- or carboxyl-terminal extensions, such as an amino-terminal methionine residue, a small linker peptide of up to about 20-25 residues, or an affinity tag.

In addition to the 20 standard amino acids, non-standard amino acids (such as 4-hydroxyproline, 6-N-methyl lysine, 2-aminoisobutyric acid, isovaline and α-methyl serine) may be substituted for amino acid residues of the polypeptides of the present invention. A limited number of non-conservative amino acids, amino acids that are not encoded by the genetic code, and unnatural amino acids may be substituted for polypeptide amino acid residues. The polypeptides of the present invention can also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethyl homo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for amino acid residues of polypeptides of the present invention.

Essential amino acids in the polypeptides of the present invention can be identified according to procedures known in the art, such as site-directed mutagenesis or alanine-scanning mutagenesis (Cunningham and Wells, Science 244: 1081-5, 1989). Sites of biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related components (e.g. the translocation or protease components) of the polypeptides of the present invention.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

SEQUENCE INFORMATION
Human SDHA amino acid sequence
SEQ ID NO: 1
MSGVRGLSRLLSARRLALAKAWPTVLQTGTRGFHFTVDGNKRASAKVSDSISAQYPVVDHEFDAVVVGAGGAGL
RAAFGLSEAGFNTACVTKLFPTRSHTVAAQGGINAALGNMEEDNWRWHFYDTVKGSDWLGDQDAIHYMTEQA
PAAVVELENYGMPFSRTEDGKIYQRAFGGQSLKFGKGGQAHRCCCVADRTGHSLLHTLYGRSLRYDTSYFVEYFAL
DLLMENGECRGVIALCIEDGSIHRIRAKNTVVATGGYGRTYFSCTSAHTSTGDGTAMITRAGLPCQDLEFVQFHPTG
IYGAGCLITEGCRGEGGILINSQGERFMERYAPVAKDLASRDVVSRSMTLEIREGRGCGPEKDHVYLQLHHLPPEQL
ATRLPGISETAMIFAGVDVTKEPIPVLPTVHYNMGGIPTNYKGQVLRHVNGQDQIVPGLYACGEAACASVHGANR
LGANSLLDLVVFGRACALSIEESCRPGDKVPPIKPNAGEESVMNLDKLRFADGSIRTSELRLSMQKSMQNHAAVFR
VGSVLQEGCGKISKLYGDLKHLKTFDRGMVWNTDLVETLELQNLMLCALQTIYGAEARKESRGAHAREDYKVRIDE
YDYSKPIQGQQKKPFEEHWRKHTLSYVDVGTGKVTLEYRPVIDKTLNEADCATVPPAIRSY
Human SDHA nucleic acid sequence
SEQ ID NO: 2
GCAGACATGTCGGGGGTCCGGGGCCTGTCGCGGCTGCTGAGCGCTCGGCGCCTGGCGCTGGCCAAGGCGT
GGCCAACAGTGTTGCAAACAGGAACCCGAGGTTTTCACTTCACTGTTGATGGGAACAAGAGGGCATCTGC
TAAAGTTTCAGATTCCATTTCTGCTCAGTATCCAGTAGTGGATCATGAATTTGATGCAGTGGTGGTAGGC
GCTGGAGGGGCAGGCTTGCGAGCTGCATTTGGCCTTTCTGAGGCAGGGTTTAATACAGCATGTGTTACCA
AGCTGTTTCCTACCAGGTCACACACTGTTGCAGCACAGGGAGGAATCAATGCTGCTCTGGGGAACATGGA
GGAGGACAACTGGAGGTGGCATTTCTACGACACCGTGAAGGGCTCCGACTGGCTGGGGGACCAGGATGCC
ATCCACTACATGACGGAGCAGGCCCCCGCCGCCGTGGTCGAGCTAGAAAATTATGGCATGCCGTTTAGCA
GAACTGAAGATGGGAAGATTTATCAGCGTGCATTTGGTGGACAGAGCCTCAAGTTTGGAAAGGGCGGGCA
GGCCCATCGGTGCTGCTGTGTGGCTGATCGGACTGGCCACTCGCTATTGCACACCTTATATGGACGGTCT
CTGCGATATGATACCAGCTATTTTGTGGAGTATTTTGCCTTGGATCTCCTGATGGAGAACGGGGAGTGCC
GTGGTGTCATCGCACTGTGCATAGAGGACGGGTCCATCCATCGCATAAGAGCAAAGAACACTGTTGTTGC
CACAGGAGGCTACGGGCGCACCTACTTCAGCTGCACGTCTGCCCACACCAGCACTGGCGACGGCACGGCC
ATGATCACCAGGGCAGGCCTTCCTTGCCAGGACCTAGAGTTTGTTCAGTTCCACCCTACAGGCATATATG
GTGCTGGTTGTCTCATTACGGAAGGATGTCGTGGAGAGGGAGGCATTCTCATTAACAGTCAAGGCGAAAG
GTTTATGGAGCGATACGCCCCTGTCGCGAAGGACCTGGCGTCTAGAGATGTGGTGTCTCGGTCCATGACT
CTGGAGATCCGAGAAGGAAGAGGCTGTGGCCCTGAGAAAGATCACGTCTACCTGCAGCTGCACCACCTAC
CTCCAGAGCAGCTGGCCACGCGCCTGCCTGGCATTTCAGAGACAGCCATGATCTTCGCTGGCGTGGACGT
CACGAAGGAGCCGATCCCTGTCCTCCCCACCGTGCATTATAACATGGGCGGCATTCCCACCAACTACAAG
GGGCAGGTCCTGAGGCACGTGAATGGCCAGGATCAGATTGTGCCCGGCCTGTACGCCTGTGGGGAGGCCG
CCTGTGCCTCGGTACATGGTGCCAACCGCCTCGGGGCAAACTCGCTCTTGGACCTGGTTGTCTTTGGTCG
GGCATGTGCCCTGAGCATCGAAGAGTCATGCAGGCCTGGAGATAAAGTCCCTCCAATTAAACCAAACGCT
GGGGAAGAATCTGTCATGAATCTTGACAAATTGAGATTTGCTGATGGAAGCATAAGAACATCGGAACTGC
GACTCAGCATGCAGAAGTCAATGCAAAATCATGCTGCCGTGTTCCGTGTGGGAAGCGTGTTGCAAGAAGG
TTGTGGGAAAATCAGCAAGCTCTATGGAGACCTAAAGCACCTGAAGACGTTCGACCGGGGAATGGTCTGG
AACACGGACCTGGTGGAGACCCTGGAGCTGCAGAACCTGATGCTGTGTGCGCTGCAGACCATCTACGGAG
CAGAGGCACGGAAGGAGTCACGGGGCGCGCATGCCAGGGAAGACTACAAGGTGCGGATTGATGAGTACGA
TTACTCCAAGCCCATCCAGGGGCAACAGAAGAAGCCCTTTGAGGAGCACTGGAGGAAGCACACCCTGTCC
TATGTGGACGTTGGCACTGGGAAGGTCACTCTGGAATATAGACCCGTGATCGACAAAACTTTGAACGAGG
CTGACTGTGCCACCGTCCCGCCAGCCATTCGCTCCTACTGATGAGACAAGATGTGGTGATGACAGAATCA
GCTTTTGTAATTATGTATAATAGCTCATGCATGTGTCCATGTCATAACTGTCTTCATACGCTTCTGCACT
CTGGGGAAGAAGGAGTACATTGAAGGGAGATTGGCACCTAGTGGCTGGGAGCTTGCCAGGAACCCAGTGG
CCAGGGAGCGTGGCACTTACCTTTGTCCCTTGCTTCATTCTTGTGAGATGATAAAACTGGGCACAGCTCT
TAAATAAAATATAAATGAAC
Human SDHB amino acid sequence
SEQ ID NO: 3
MAAVVALSLRRRLPATTLGGACLQASRGAQTAAATAPRIKKFAIYRWDPDKAGDKPHMQTYEVDLNKCGPMVLD
ALIKIKNEVDSTLTFRRSCREGICGSCAMNINGGNTLACTRRIDTNLNKVSKIYPLPHMYVIKDLVPDLSNFYAQYKSI
EPYLKKKDESQEGKQQYLQSIEEREKLDGLYECILCACCSTSCPSYWWNGDKYLGPAVLMQAYRWMIDSRDDFTE
ERLAKLQDPFSLYRCHTIMNCTRTCPKGLNPGKAIAEIKKMMATYKEKKASV
Human SDHB nucleic acid sequence
SEQ ID NO: 4
GGCCTCCCACTTGGTTGCTCGTACGCGGCTAGTGGGTCCTCAGTGGATGTAGGCTGGGCG
CCGCGATGTTCGACGGGACACCGGCGGAGAGCGACCTCGGGGTTAAGGGGTGGGGCTGGA
CGTCAGGAGCCAAGATGGCGGCGGTGGTCGCACTCTCCTTGAGGCGCCGGTTGCCGGCCA
CAACCCTTGGCGGAGCCTGCCTGCAGGCCTCCCGAGGAGCCCAGACAGCTGCAGCCACAG
CTCCCCGTATCAAGAAATTTGCCATCTATCGATGGGACCCAGACAAGGCTGGAGACAAAC
CTCATATGCAGACTTATAAGGTTGACCTTAATAAATGTGGCCCCATGGTATTGGATGCTT
TAATCAAGATTAAGAATGAAGTTGACTCTACTTTGACCTTCCGAAGATCATGCAGAGAAG
GCATCTGTGGCTCTTGTGCAATGAACATCAATGGAGGCAACACTCTAGCTTGCACCCGAA
GGATTGACACCAACCTCAATAAGGTCTCAAAAATCTACCCTCTTCCACACATGTATGTGA
TAAAGGATCTTGTTCCCGATTTGAGCAACTTCTATGCACAGTACAAATCCATTGAGCCTT
ATTTGAAGAAGAAGGATGAATCTCAGGAAGGCAAGCAGCAGTATCTGCAGTCCATAGAAG
AGCGTGAGAAACTGGACGGGCTCTACGAGTGCATTCTCTGTGCCTGCTGTAGCACCAGCT
GCCCCAGCTACTGGTGGAACGGAGACAAATATCTGGGGCCTGCAGTTCTTATGCAGGCCT
ATCGCTGGATGATTGACTCCAGAGATGACTTCACAGAGGAGCGCCTGGCCAAGCTGCAGG
ACCCATTCTCTCTATACCGCTGCCACACCATCATGAACTGCACAAGGACCTGTCCTAAGG
GTCTGAATCCAGGGAAAGCTATTGCAGAGATCAAGAAAATGATGGCAACCTATAAGGAGA
AGAAAGCTTCAGTTTAACTGTTTCCATGCTAAACATGATTTATAACCAGCTCAGAGCTGA
ACATAATTTATATCTAATTTGAGTTCCTTTAAAGATCTTGGTTTTCCATGAATACAGCAT
GTATAATAAAAATTTTAAGA
Human SDHC amino acid sequence
SEQ ID NO: 5
MAALLLRHVGRHCLRAHFSPQLCIRNAVPLGTTAKEEMERFWNKNIGSNRPLSPHITIYSWSLPMAMSICHRGTGI
ALSAGVSLFGMSALLLPGNFESYLELVKSLCLGPALIHTAKFALVFPLMYHTWNGIRHLMWDLGKGLKIPQLYQSGV
VVLVLTVLSSMGLAAM
Human SDHC nucleic acid sequence
SEQ ID NO: 6
AGAGAACATTCCTCCTACCATCACCACACAATAGAACCTGATTTTCTGTCATTGGAATAA
GTGGATAATAGGTTCCATACTGTGGGTTTTGAGAAGGGTAAAGGTGGGGCATAAGGGTAG
AAGCGCTTTTCTCTAGAATCATGCTGAGAGGAGATATCTATTCCTTTAGGTAGAATTACT
TTNTGAGACAGGAACTGTTAATGTCCTATTTACTGAAATTCCTTTTTTTTTTTTTTGCTT
TGTCCACAGATGTGGGACCTAGGAAAAGGCCTGAAGATTCCCCAGCTATACCAGTCTGGA
GTGGTTGTCCTGGTTCTTACTGTGTTGTCCTCTATGGGGCTGGCAGCCATGTGAAGAAAG
GAGGCTCCCAGCATCATCTTCCTACACATTATTACATTCACCCATCTTTCTGTTTGTCAT
TCTTATCTCCAGCCTGGGAAAAGTTCTCCTTATTTGTTTAGATCCTTTTGTATTTTCAGA
TCTCCTTGGAGCAGTAGAGTACCTGGTAGACCATAATAGTGGAAAAGGGTCTAGTTTTCC
CCTTGTTTCTAAAGATGAGGTGGCTGCAAAAACTCCCCTTTTTTGCCCACAGCTTGCCTA
CTCTCGGCCTAGAAGCAGTTATTCTCTCTCCATATTGGGCTTTGATTTGTGCTGAGGGTC
AGCTTTTGGCTCCTTCTTCCTGAGACAGTGGAAACAATGCCAGCTCTGTGGCTTCTGCCC
TGGGGATGGGCCGGGTTGGGGGGTGGGTTGGTGAGGCTTTGGGTGCCACTGCCTGTGGGT
TGCTGGCTTAAAGGACAATTCTCTTCATTGGTGAGAGCCCAGGCCATTAACACCTACACA
GTGTTATTGAAAGAAGAGAGGTGGGGGTGGAGGGGAATTAGTCTGTCCCAGCTAGAGGGA
GATAAAGAGGGCTAGTTAGTTCTTGGAGCAGCTGCTTTTGAGGAGAAAATATATAGCTTT
GGACACGAGGAAGATCTAGAAAATTATCATTGAACATATTAATGGTTATTTCTTTTTCTT
GGATTTCCAGAAAAGCCTCTTAATTTTATGCTTTCTCATCGAAGTAATGTACCCTTTTTT
TCTGAAACTGAATTAAATACTCATTTT
Human SDHD amino acid sequence
SEQ ID NO: 7
MAVLWRLSAVCGALGGRALLLRTPVVRPAHISAFLQDRPIPEWCGVQHIHLSPSHHSGSKAASLHWTSERVVSVLL
LGLLPAAYLNPCSAMDYSLAAALTLHGHWGLGQVVTDYVHGDALQKAAKAGLLALSALTFAGLCYFNYHDVGICK
AVAMLWKL
Human SDHD nucleic acid sequence
SEQ ID NO: 8
ATGGCGGTTCTCTGGAGGCTGAGTGCCGTTTGCGGTGCCCTAGGAGGCCGAGCTCTGTTG
CTTCGAACTCCAGTGGTCAGACCTGCTCATATCTCAGCATTTCTTCAGGACCGACCTATC
CCAGAATGGTGTGGAGTGCAGCACATACACTTGTCACCGAGCCACCATTCTGGCTCCAAG
GCTGCATCTCTCCACTGGACTAGCGAGAGGGTTGTCAGTGTTTTGCTCCTGGGTCTGCTT
CCGGCTGCTTATTTGAATCCTTGCTCTGCGATGGACTATTCCCTGGCTGCAGCCCTCACT
CTTCATGGTCACTGGGGCCTTGGACAAGTTGTTACTGACTATGTTCATGGGGATGCCTTG
CAGAAAGCTGCCAAGGCAGGGCTTTTGGCACTTTCAGCTTTAACCTTTGCTGGGCTTTGC
TATTTCAACTATCACGATGTGGGCATCTGCAAAGCTGTTGCCATGCTGTGGAAGCTCTAG
Human ACOD1 amino acid sequence
SEQ ID NO: 9
MMLKSITESFATAIHGLKVGHLTDRVIQRSKRMILDTLGAGFLGTTTEVFHIASQYSKIYSSNISSTVWGQPDIRLPPT
YAAFVNGVAIHSMDFDDTWHPATHPSGAVLPVLTALAEALPRSPKFSGLDLLLAFNVGIEVQGRLLHFAKEANDM
PKRFHPPSVVGTLGSAAAASKFLGLSSTKCREALAIAVSHAGAPMANAATQTKPLHIGNAAKHGIEAAFLAMLGLQ
GNKQVLDLEAGFGAFYANYSPKVLPSIASYSWLLDQQDVAFKRFPAHLSTHWVADAAASVRKHLVAERALLPTDYI
KRIVLRIPNVQYVNRPFPVSEHEARHSFQYVACAMLLDGGITVPSFHECQINRPQVRELLSKVELEYPPDNLPSFNIL
YCEISVTLKDGATFTDRSDTFYGHWRKPLSQEDLEEKFRANASKMLSWDTVESLIKIVKNLEDLEDCSVLTTLLKGPS
PPEVASNSPACNNSITNLS
Human ACOD1 nucleic acid sequence
SEQ ID NO: 10
ATGATGCTCAAGTCTATCACAGAAAGCTTTGCCACAGCAATCCATGGCTTGAAAGTGGGACACCTGACAG
ATCGTGTTATTCAGAGGAGCAAGAGGATGATTCTAGACACTCTGGGTGCTGGGTTCCTGGGAACCACTAC
GGAAGTGTTTCACATAGCCAGCCAATATAGCAAGATCTACAGTTCCAACATATCCAGCACTGTTTGGGGT
CAGCCAGACATCAGGCTCCCGCCCACATATGCTGCTTTTGTGAACGGTGTGGCTATTCACTCCATGGATT
TTGATGACACGTGGCACCCTGCCACCCACCCTTCTGGGGCTGTCCTTCCTGTCCTCACAGCTTTAGCAGA
AGCCCTGCCAAGGAGTCCAAAGTTTTCTGGCCTTGACCTGCTGCTGGCTTTCAATGTTGGTATTGAAGTG
CAAGGCCGATTACTGCATTTCGCCAAGGAGGCCAATGACATGCCAAAGAGATTCCATCCCCCTTCCGTGG
TAGGAACGTTGGGTAGTGCTGCTGCTGCATCCAAGTTTTTAGGACTTAGCTCGACAAAGTGCCGAGAAGC
TCTGGCCATTGCTGTTTCCCATGCTGGGGCACCCATGGCCAATGCTGCCACCCAGACCAAGCCCCTCCAC
ATTGGCAATGCTGCCAAGCATGGGATAGAAGCTGCATTTTTGGCAATGTTGGGTCTCCAAGGAAACAAGC
AGGTCTTGGACTTGGAGGCAGGATTTGGGGCCTTTTATGCCAACTATTCCCCAAAAGTCCTTCCAAGCAT
AGCTTCCTACAGTTGGCTGCTGGACCAGCAGGACGTGGCCTTTAAGCGTTTTCCTGCACATTTATCTACC
CACTGGGTGGCAGACGCAGCTGCATCTGTGAGAAAGCACCTTGTAGCAGAGAGAGCCCTGCTTCCAACTG
ACTACATTAAGAGAATTGTGCTCAGGATACCAAATGTCCAGTATGTAAACAGGCCCTTTCCAGTTTCGGA
GCATGAAGCCCGTCATTCATTCCAGTATGTGGCCTGTGCCATGCTGCTTGATGGTGGCATCACTGTCCCC
TCATTCCATGAATGCCAGATCAACAGGCCACAGGTGAGAGAGCTGCTCAGTAAGGTGGAGCTGGAGTACC
CTCCGGACAACTTGCCAAGCTTCAACATACTGTACTGTGAAATAAGTGTCACCCTCAAGGATGGAGCCAC
CTTCACAGATCGCTCTGATACCTTCTATGGGCACTGGAGAAAACCACTGAGCCAGGAGGACCTAGAGGAA
AAGTTCAGAGCCAATGCCTCCAAGATGCTGTCCTGGGACACAGTGGAAAGCCTTATAAAGATAGTCAAAA
ATCTAGAAGACCTAGAAGACTGTTCTGTGTTAACTACACTTCTCAAAGGACCCTCTCCACCAGAGGTAGC
TTCAAACTCTCCAGCATGTAATAATTCTATCACAAATCTCTCC

### EXAMPLES

The invention will be further clarified by the following examples, which are intended to be purely exemplary of the invention and are in no way limiting.

### Materials and Methods

### Experimental Model and Subject Details

### Human Bronchoalveolar Lavage

Bronchoscopy of the right middle lobe was performed after informed consent as approved by an external Research Ethics Committee for ILD subjects (Ref. Nos. 10/H0720/12 and 15/SC0101) and healthy control subjects (Ref. No. 15-LO-1399) according to the Royal Brompton Hospital protocol (Royal Brompton & Harefield NHS Foundation Trust, 2016). Bronchoscopies were performed with subjects under a light sedation with midazolam in combination with local anesthesia with lidocaine. Four 60-ml aliquots of warmed sterile saline were instilled in the right middle lung lobe and aspirated by syringe and lavage aliquots collected after each instillation were pooled for each patient. Volume and BAL appearance were recorded for all samples.

### Cell Culture

Primary human lung fibroblasts were isolated from lung resections of patients undergoing lung cancer surgery or lung transplantation performed after informed consent as approved by an external Research Ethics Committee (REC 15/SC0101) according to the Royal Brompton Hospital protocol (Royal Brompton & Harefield NHS Foundation Trust, 2016) and cultured in complete Dulbecco's modified eagle medium (10% FBS, 100 U/ml penicillin/streptomycin) (Gibco, ThermoFisher) to passage four. Human bronchial epithelial cells (HBE) were obtained from bronchial brushings during the bronchoscopy after informed consent and approved by an external Research Ethics Committee for ILD subjects (Ref. Nos. 10/H0720/12 and 15/SC0101). HBEs were cultured in bronchia.1 e thelial growth (BEGM) medium (Lonza) to passage four. MACS enriched human AM were cultured in complete RPMI (10% FBS, 100U/ml penicillin/streptomycin, Gibco, ThermoFisher) for 24 hrs. Fibroblasts and AM were cultured with 10 mM itaconate in complete medium for 24hrs prior to cell lysis in RLT buffer (QIAGEN) containing 1% 2-Mercaptoethanol (Sigma Aldrich).

### Mice

Acod1^{-/-} (C57BL/6NJ-Acod1\hat{ }(em1J)/J, JAX stock number 029340) mice and littermate controls were bred on a C57BL/6 background. Unless otherwise stated, all mice were between 8 and 12 weeks of age. Mice were housed in specific-pathogen-free conditions and given food and water ad libitum. All procedures were approved by the United Kingdom Home Office and conducted in strict accordance with the Animals (Scientific Procedures) Act 1986. The Imperial College London Animal Welfare and Ethical Review Body (AWERB) approved this protocol. All surgery was performed under ketamine and sodium pentobarbital anaesthesia and all efforts were made to minimize suffering. Mice were administered either 0.05U (1U/ml solution dissolved in PBS) of bleomycin sulphate (Sigma Aldrich) or 50 μl PBS via the oropharyngeal route at day 0 and culled after 7, 21 or 42 days. For therapeutic experiments mice were administered 0.25 mg/kg (1 mM solution dissolved in PBS, 50 μl) itaconic acid (Sigma Aldrich) or PBS via the oropharyngeal route twice a week, beginning 10 days after of bleomycin administration.

### Subject Demographics

Seventy-six IPF patients and 17 control subjects were recruited. Demographic and clinicopathological features are detailed in Tables 1 and 2. Healthy volunteers had no self-reported history of lung disease, an absence of infection within the last 6 months and normal spirometry.

### Human AM Isolation

1×10⁷ BAL cells were stained with anti-CD206 (Biolegend) and Human TruStain FcX block (Biolegend) for 15 minutes at 4° C. in 0.5% FBS/2 mM EDTA in PBS prior to incubation with MACS anti-Cy7 microbeads (Miltenyi Biotec) for 15 minutes at 4° C. Cells were enriched in MACS magnetic separation column (Miltenyi Biotec) and purity was confirmed on a representative subset (n=29) by flow cytometry.

### Single Cell RNA Sequencing

Viable cryopreserved BAL cells were sorted on a BD Influx sorter (Becton Dickinson) as previously described (Byrne et al., 2020) and retained on ice. Briefly, cells at a concentration of 800-1,000 cells/μl were loaded onto 10× Genomics single cell 3′ chips along with the RT mastermix (Chromium Single Cell 3′ Library, v2, PN-120233, 10× Genomics) according to manufacturer's instructions to generate single-cell gel beads in emulsion. RT was performed using a C1000 Touch Thermal Cycler with a Deep Well Reaction Module (Bio-Rad; 55° C. for 2 h; 85° C. for 5 min; hold 4° C.). DynaBeads (MyOne Silane Beads, Thermo Fisher Scientific) and SPRIselect beads (Beckman Coulter) were used to purify and recover cDNA., which was subsequently amplified (98° C. for 3 min; 12 times −98° C. for 15 s, 67° C. for 20 s, 72° C. for 60 s); 72° C. for 60 s; hold 4° C.). Amplified cDNA was sheared to ˜200 base pairs with a Covaris S2 instrument using the manufacturer's recommended parameters. Sequencing libraries were generated with unique sample indices and sequenced on a Illumina NewtSeq 500 (NextSeq control software v2.0.2/ Real Time Analysis v2.4.11) using a 150-cycle NextSeq 500/550 High Output Reagent Kit v2 (FC-404-2002; Illumina) in stand-alone mode as follows: 98 bp (read 1), 14 bp (17 index), 8 bp (15 index), and 485 10 bp (read 2).

The Cell Ranger Single Cell Software Suite (10X Genomics, v2.0.0) was used to process the sequencing data into transcript count tables. Raw base call files were demultiplexed using the Cell Ranger mkfastq programme into sample-specific FASTQ files, which were then processed using the Cell Ranger count pipeline. Subsequent analysis was performed as described previously (Byrne et al., 2020).

### Determination of Itaconate by Gas Chromatography/Mass Spectrometry

Freeze dried BAL samples were spiked with d 3 -labelled methylmalonic acid (d3-MMA, synthesized in house) and derivatized with 30 μl methoxyamine hydrochloride (Sigma-Aldrich, 20 mg/ml in pyridine, 40° C. for 20 min) to modify any carbonyls (multi-component method). After cooling, 70 μl of N,O-bis(trimethylsilyl)trifluoroacetamide containing 1% trimethylchlorosilane (BSTFA Sigma-Aldrich) were added and the mixture incubated for 30 minutes at 60° C. to effect trimethylsilylation of the hydroxy functions. Finally, the supernatants from centrifuged reaction mixtures were transferred to injection vials. GC/MS analysis was performed on an Agilent 6890 gas chromatograph coupled to a 5973 MSD quadrupole mass spectrometer. Samples were injected in splitless mode with the inlet maintained at 280° C. Separation of the derivatives was performed on a DB-1701 capillary column 30 m×250μm×0.25 μm (Agilent Technologies) using a three-stage temperature program to optimize the separation. Mass spectral data was acquired by selected ion monitoring (SIM) of m/z 259 (quantifier) and m/z 215 (qualifier) at approx. 6 min retention time. A five-level calibration plot was constructed over the concentration range 0-16 ng/ml. Quantitation was achieved by interpolation using the regression equation of the calibration curve. All data processing and concentration calculations were performed using Agilent MassHunter (v. B.07.01) software.

### Murine Lung Function Assessment

Lung function measurements were performed using the Flexivent system (Scireq, Montreal, Canada). After induction of anaesthesia with an i.p. injection of Pentobarbitone (50 mg/Kg, Sigma, UK) and i.m. injection of Ketamine (200 mg/Kg) (Fortdodge Animal Health Ltd, Southampton, UK), mice were tracheotomised and attached to the Flexivent ventilator via a blunt-ended 19-gauge needle. Mice were ventilated using the following settings; tidal volume of 7 ml/Kg body weight, 150 breaths/minute; positive end-expiratory pressure approximately 2 cm H₂O. Standardisation of lung volume history was done by performing two deep inflations. Subsequently, measurements of dynamic resistance, dynamic elastance and dynamic compliance were determined using the snapshot-150 perturbation, a single frequency sinusoidal waveform. Resultant data was fitted using multiple linear regression to the single compartment model to determine the above parameters.

### Murine BAL and Lung Cell Recovery

In order to obtain BAL, the airways of the mice were lavaged three times with 0.4 ml of PBS via a tracheal cannula. BAL fluid was centrifuged (700 X g, 5 min, 4° C.); cells were resuspended in 0.5 ml complete media (RPMI+10% fetal calf serum [FCS], 2 mM L-glutamine, 100 Wml penicillin/streptomycin). Cells were counted and pelleted onto glass slides by cytocentrifugation (5×10 4 cells/slide). To disaggregate cells from lung tissue, one finely chopped left lobe of lung was incubated at 37° C. for 1 h in digest reagent (0.15 mg/ml collagenase type D, 25 μg/l DNase type I) in complete RPMI media. The recovered cells were filtered through a 70-μm nylon sieve, washed twice, resuspended in 1 ml complete media, and counted in a haemocytometer prior to cytocentrifugation; lung cell counts are quoted as total cell number/ml of this suspension.

### Flow Cytometry

Cells were stained with near IR fixable live/dead (ThermoFisher) for 10 minutes in PBS prior to staining for extracellular antigens in 1% FBS/2.5% HEPES/0.2% EDTA in PBS for 20 minutes at 4° C.

For assessment of mitochondrial superoxide, cells were stained with 5 uM MitoSOX Red (ThermoFisher) in PBS for 10 minutes at 37° C. Cells were then washed and fixed using IC fix kit (eBioscience). All antibodies were purchased from Biolegend. Data was acquired with Fortessa II and cell sorting on Aria III (BD Biosciences) and analysis was performed in Flowjo software, using FMO's for each antibody.

### Adoptive Transfer of FACS Sorted Mo-AMs

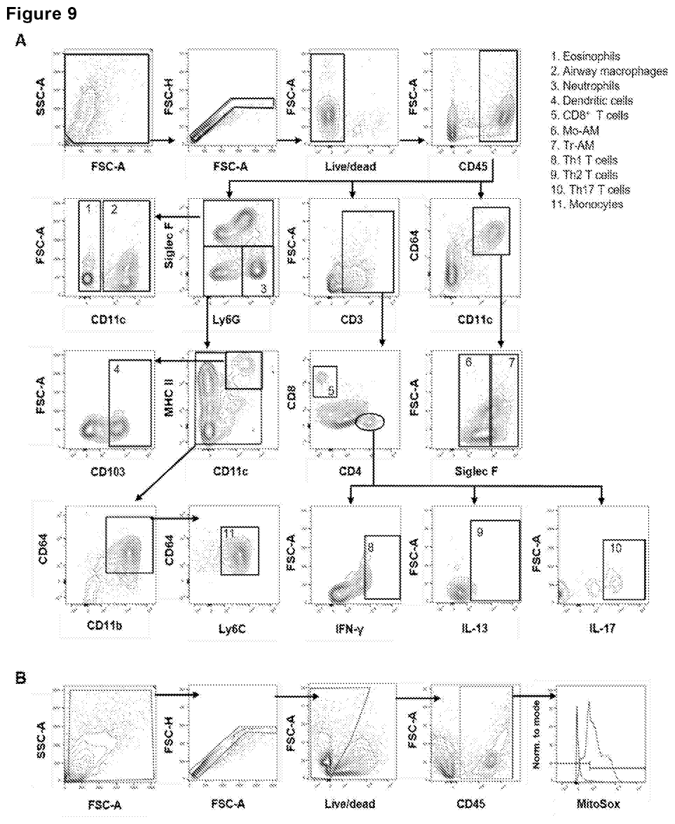

Female WT or Acod1−/− mice were dosed with 0.05U bleomycin via the oropharyngeal route and lavaged at day 7 post bleomycin to obtain monocyte-recruited AMs (Mo-AMs). Cells recovered from bronchoaleolar lavage were stained with extracellular antibodies as described above and live, CD45⁺, CD64⁺, CD11c⁺, SigF^{int} Mo-AMs were isolated by FACS sorting as shown in the gating strategy in FIG. 9. Subsequently, 50,000 WT or Acod1^{-/-} Mo-AMs were administered via the oropharyngeal route to male Acod1^{-/-} mice, which had been dosed with bleomycin 7 days prior. Mice were sacrificed at day 21 post initial bleomycin exposure.

### Hydroxyproline Assay

Hydroxyproline was measured using 10 mg of tissue from the inferior lobe of murine samples using a Hydroxyproline Assay Kit (Sigma Aldrich), as per manufacturer's instructions and fold change of bleomycin/PBS groups was calculated.

### Histology

Paraffin-embedded sections (4 μm) of lungs (apical lobe) were stained with hematoxylin/eosin (H&E) and Sirius Red. For assessment of fibrosis, the semi-quantitative Ashcroft scoring system was used as previously described (Hubner et al., 2008, Biotechniques, 44(4): 507-11). All scoring and measurements were performed by 3-5 blinded independent observers.

### JULI-Stage Real-Time Cell Recording

Primary human lung fibroblasts were seeded in 96-well plate for proliferation assay (5,000 per well) or 24-well plate for wound healing assay and serum-starved overnight prior to treatment with itaconate in complete DMEM for 48-72 hrs. For wound healing assays, a standardised scratch was applied in each well using a p10 pipette tip. Images were taken in JULI-Stage system (NanoEntek) at three to five positions per well every 30 minutes and proliferation rate or wound closure were calculated using JU LI-Stage software (NanoEntek).

### Real-Time PCR

Total RNA from the post-caval lobe was extracted using the QIAGEN RNeasy Mini Kit plus (QIAGEN) or using the QIAGEN RNeasy Micro Kit plus for total RNA from cell cultures and BAL cells. Total RNA was reverse transcribed into cDNA using the High Capacity cDNA Reverse Transcription kit (Life Technologies), or GoScript reverse transcription system (Promega) for AMs, according to manufacturer's instructions. Real-time PCR was performed using fast-qPCR mastermix (Life technologies) on a Viia-7 instrument (Applied Biosciences) with Taqman primers for murine acod1, col-1α1, col3α1, col4α1, fn1, mmp2 or human acod1, cd163, fn1, IL-16, mmp1, mmp9 using actb (Life Technologies) as housekeeping gene. For analysis of murine AM fibrosis gene expression, total RNA (0.08 μg) of FACS sorted Mo-AMs or Tr-AMs was reverse transcribed into cDNA using the RT² first-strand synthesis kit as per manufacturer's instructions (QIAGEN). Gene expression of 84 genes in murine fibrosis was assessed using fast-qPCR SYBR Green Master Mix (Qiagen, Germany) and mouse fibrosis 96-well genearray (120Z, QIAGEN) on a ViiA-7 instrument. Gene expression was analysed using the QIAGEN data analysis centre.

### Seahorse Analysis

FACS sorted murine Tr-/Mo-AM (100,000 per well) were plated in a Seahorse plate coated with Cell Tak (BD Biosciences) and analysed after resting at 37° C., 5% CO₂ overnight. Oxygen Consumption Rate (OCR) and extracellular acidification rate (SCAR) were measured in XF medium (nonbuffered RPMI containing 2 mM glutamine, 1 mM pyruvate and 10 mM glucose, pH 7.4, Agilent) using the XFp extracellular flux analyser (Agilent). OCR and SCAR were measured under basal conditions and after the sequential addition of 1.5 μM Oligomycin, 2.0 μM FCCP and 0.5 μM Rotenone/Antimycin A (mito stress test, Agilent), which enabled the calculation of spare respiratory capacity from basal and maximal respiration as a result of OxPhos.

### Quantification and Statistical Analysis

Differences between non-continuous groups were compared using the Mann-Whitney U test, one-way ANOVA with Sidak's multiple comparison test or a one-sample-t test where appropriate. Kaplan-Meier analysis was used to compare time to humane endpoint in mice. Data are presented as mean±standard error of the mean (SEM). For in vivo experiments, the number of animals (n) per group are indicated. Analysis was performed using Prism software (GraphPad Software). PCA clustering was performed using the ClustVis package for R (available through Github, https://github.com/taunometsalu/ClustVis), (Metsalu et al., 2015, Nucleic Acids Research, 43(W1): W566-70). Heat-maps were generated using Morpheus software tool (https://software.broadinstitute.org/morpheus/).

### Example 1: ACOD1/Itaconate Pathway is Altered During IPF

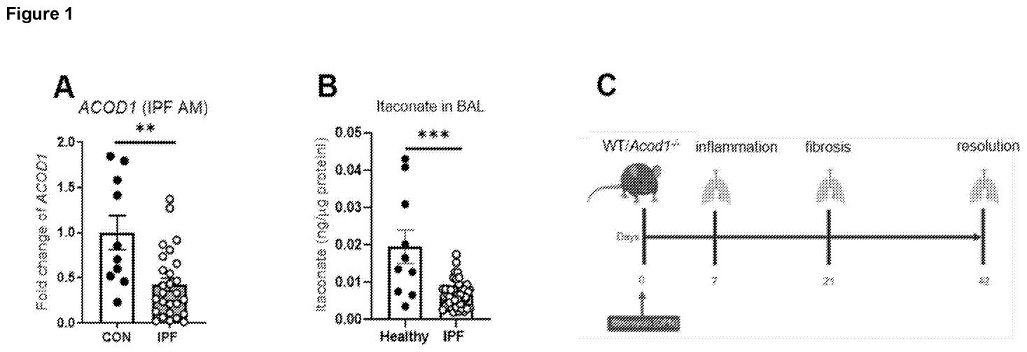

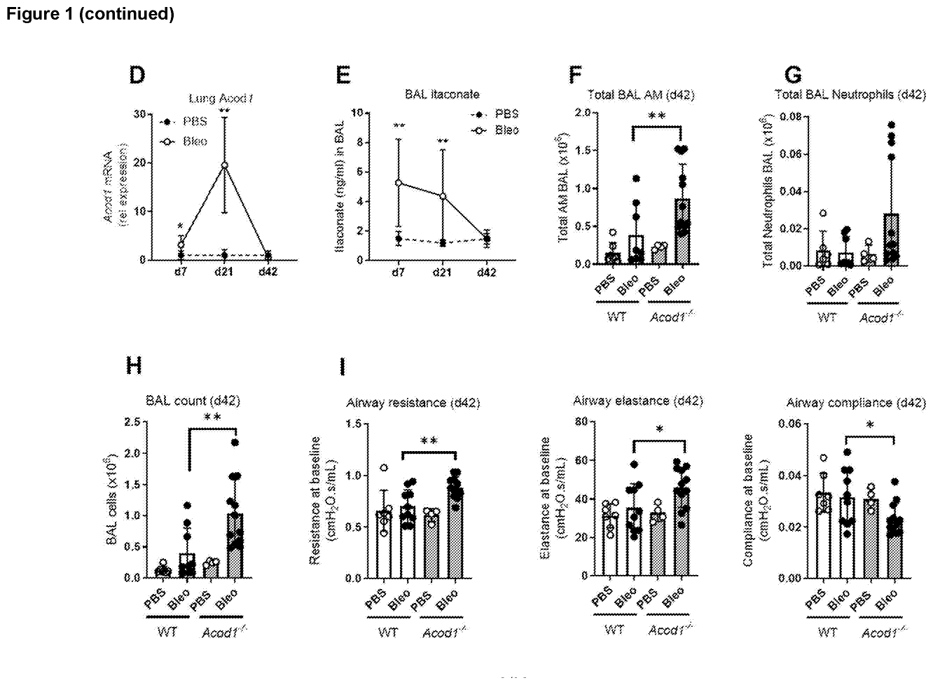

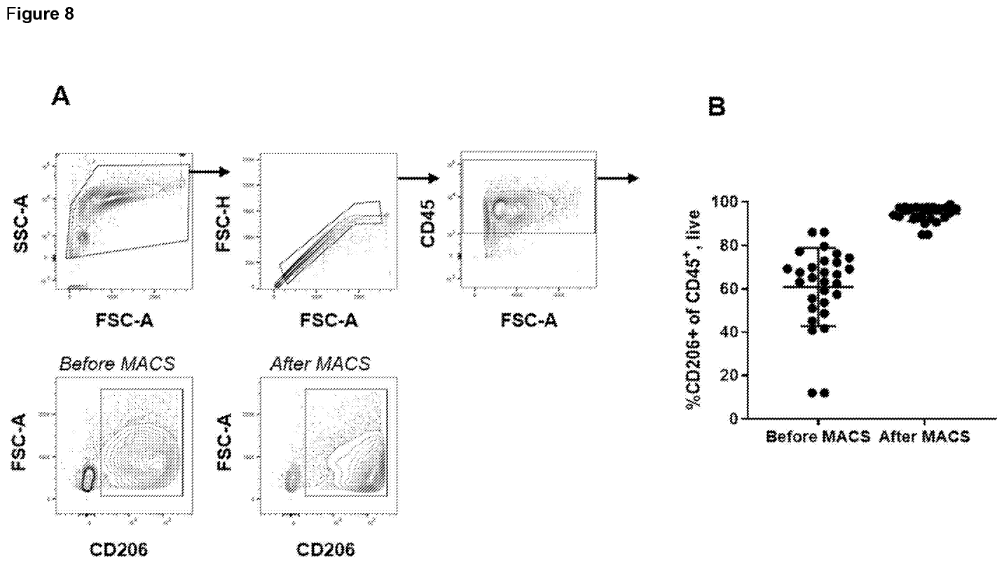

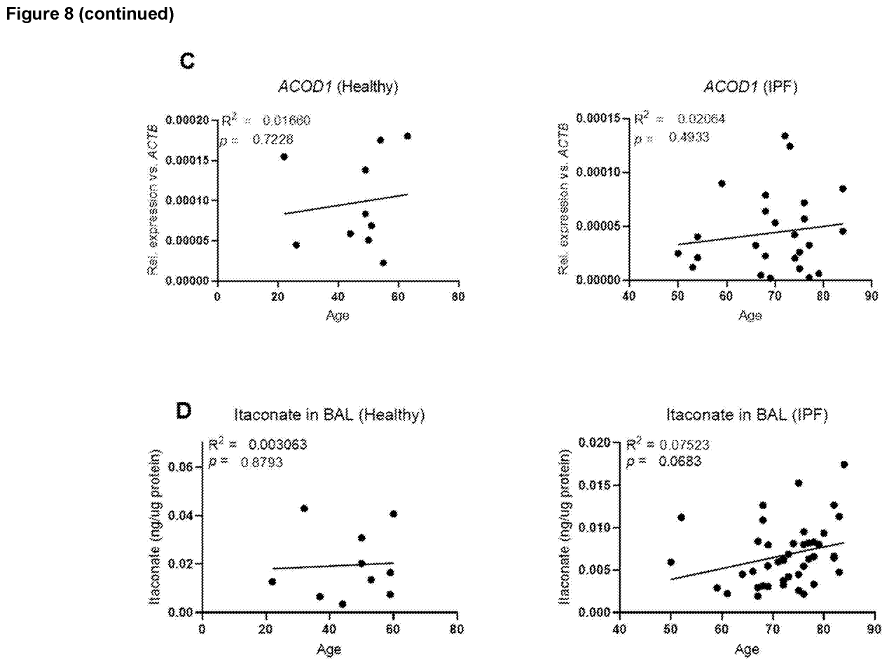

In order to determine the distribution of ACOD1 mRNA in the human lung, expression levels were assessed in primary airway macrophages (AMs), lung fibroblasts (HLF) and human bronchial epithelial cells (HBEs) from healthy volunteers and IPF patients. AMs were enriched using magnetic associated cell sorting (MACS) based on CD206 expression, as this marker has been identified as most expressed on human airway macrophages (see Tables 1 and 2 for patient demographics). ACOD1 was expressed in CD206⁺ AMs from healthy volunteers and this expression level was significantly reduced in cells from IPF, when assessed by qPCR (FIG. 1A and FIG. 8A, 8B). Furthermore, levels of BAL itaconate (normalized to total protein) were decreased in IPF patients compared to healthy controls (FIG. 1B). In contrast, we could not detect any ACOD1 transcript in HLFs or HBE's (data not shown). These results indicate that the ACOD1/itaconate axis is significantly altered in the lungs of individuals with fibrotic lung disease.

TABLE 1
Subject demographics of samples used for qPCR analysis (FIG. 1A).

| | IPF | Healthy | |
|---|---|---|---|
| | (n = 27) | (n = 10) | |
| | (Mean ± S.D.) | (Mean ± S.D.) | T-Test |
| Sex (M/F) | 21M/6F | 5M/5F | |
| Age | 69.5 ± 9.0 | 43.6 ± 11.0 | **** |
| On anti-fibrotic treatment | 41% | — | |
| Of which: | | | |
| Nintednib | 45% | | |
| Pifenidone | 55% | | |
| On steroids | — | — | |
| FEV1 | 2.3 ± 0.7 | 2.8 ± 0.8 | n.s. |
| FEV1 (predicted) | 85.4 ± 18.5 | NA | |
| FVC | 2.8 ± 0.9 | 3.5 ± 1.1 | n.s. |
| FVC (predicted | 81.8 ± 18.1 | NA | |
| DLCO (single breath) | 4.0 ± 1.7 | NA | |
| Ever smoked | 77% | 20% | |
| Of which: | | | |
| Ex-Smoker | 90% | 100% | |
| Current-smoker | 10% | | |

Table 1 shows the sex, age, drug-treatment, FEV1, FEV1% predicted, forced vital capacity (FVC), % predicted FVC and smoking status of Healthy (n=10) and IPF (n=27) samples used for qPCR of ACOD1 (FIG. 1A). Data presented as mean±S.D.

TABLE 2
Subject demographics of samples used for GC-MS analysis (FIG. 1B).

| | IPF | Healthy | |
|---|---|---|---|
| | (n = 27) | (n = 10) | |
| | (Mean ± S.D.) | (Mean ± S.D.) | T-Test |
| Sex (M/F) | 37/10 | 6/4 | |
| Age | 72.9 ± 7.7 | 46.6 ± 12.1 | **** |
| On anti-fibrotic treatment | 70% | — | |
| Of which: | | | |
| Nintednib | 36% | | |
| Pifenidone | 78% | | |
| On steroids | 8.5% | — | |
| FEV1 | 2.2 ± 0.6 | 3.0 ± 0.6 | * |
| FEV1 (predicted) | 84.6 ± 14.2 | NA | |
| FVC | 2.7 ± 0.8 | 3.7 ± 0.9 | n.s. |
| FVC (predicted | 80.6 ± 13.6 | NA | |
| DLCO (single breath) | 3.8 ± 1.4 | NA | |
| Ever smoked | 64% | 33% | |
| Of which: | | | |
| Ex-Smoker | 87% | — | |
| Current-smoker | 13% | 100% | |

Table 2 shows the sex, age, drug-treatment, FEV1, FEV1% predicted, forced vital capacity (FVC), % predicted FVC and smoking status of Healthy (n=10) and IPF (n=47) samples used for GC-MS of itaconate (FIG. 1B). Data presented as mea±S.D.

### Example 2: Acod1 Deficiency Results in Worsened Pulmonary Fibrosis in Mice

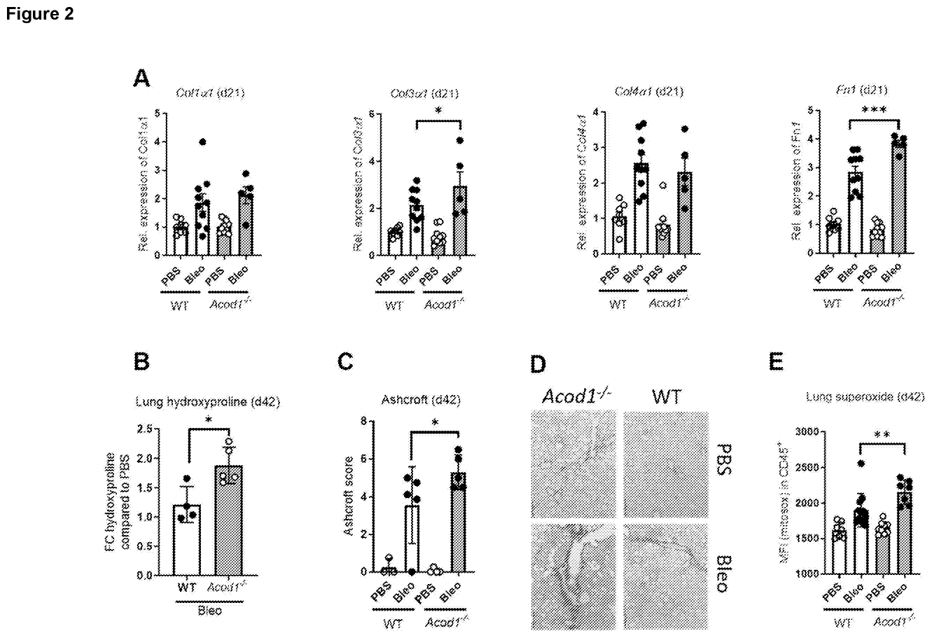

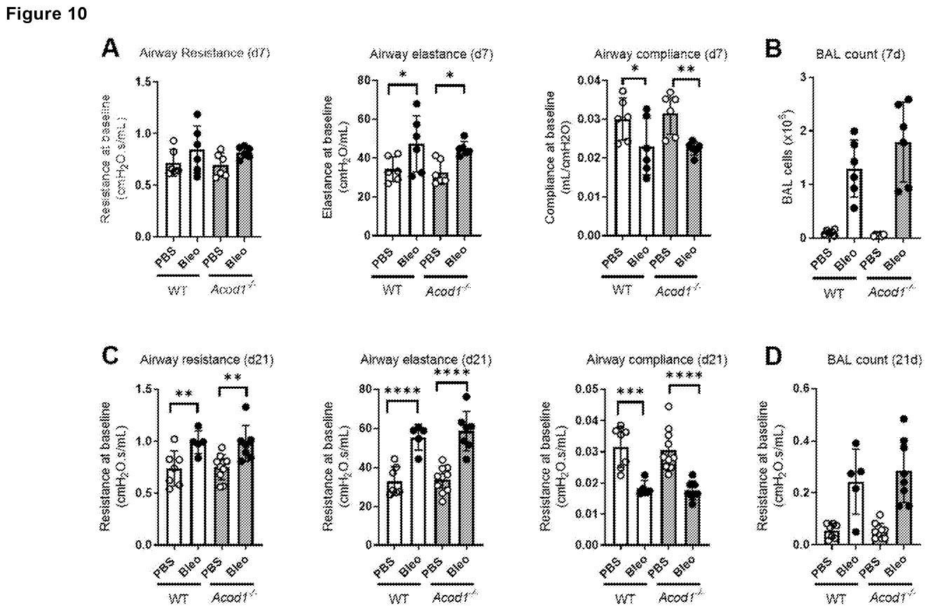

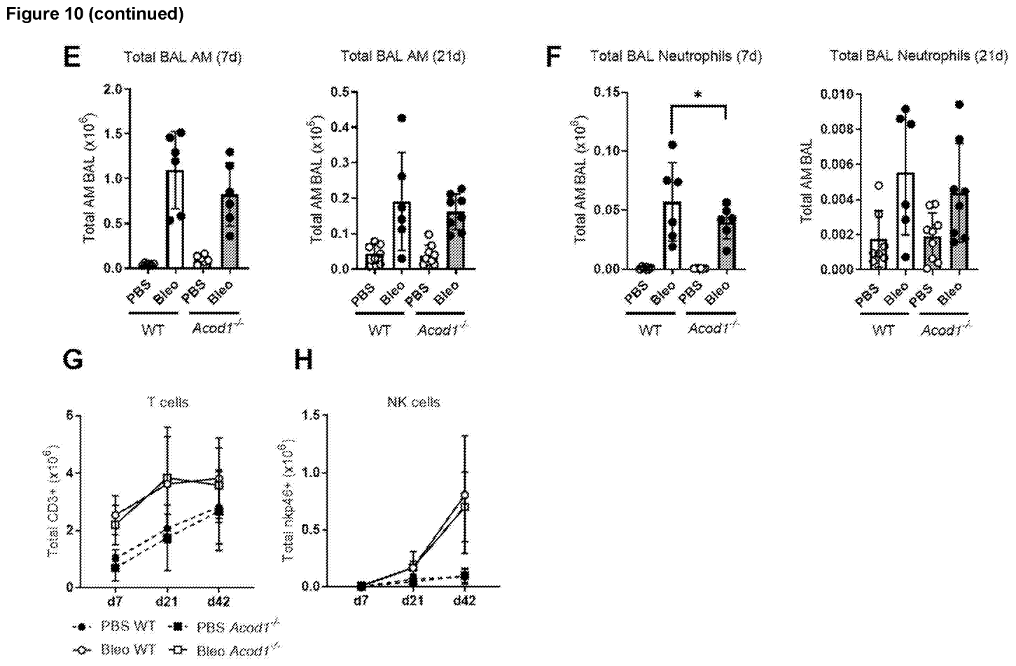

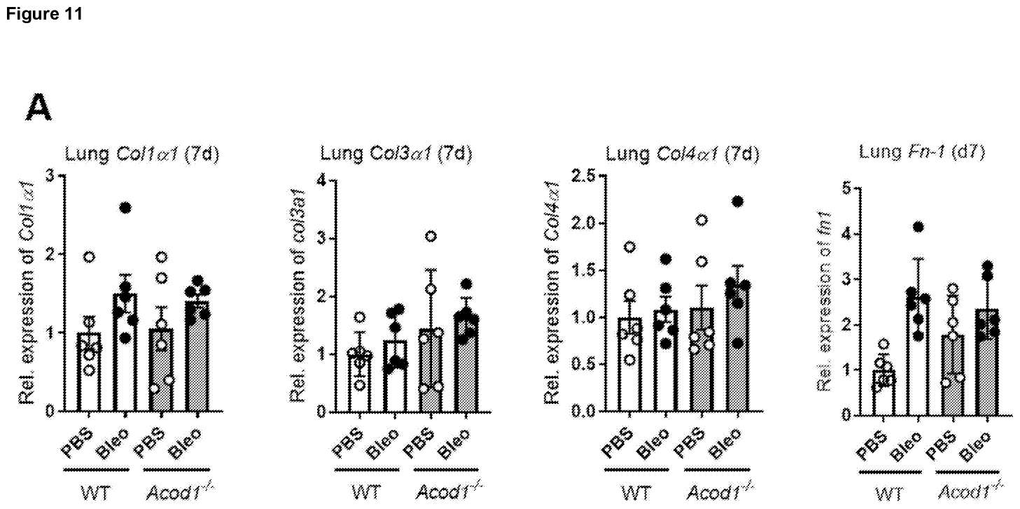

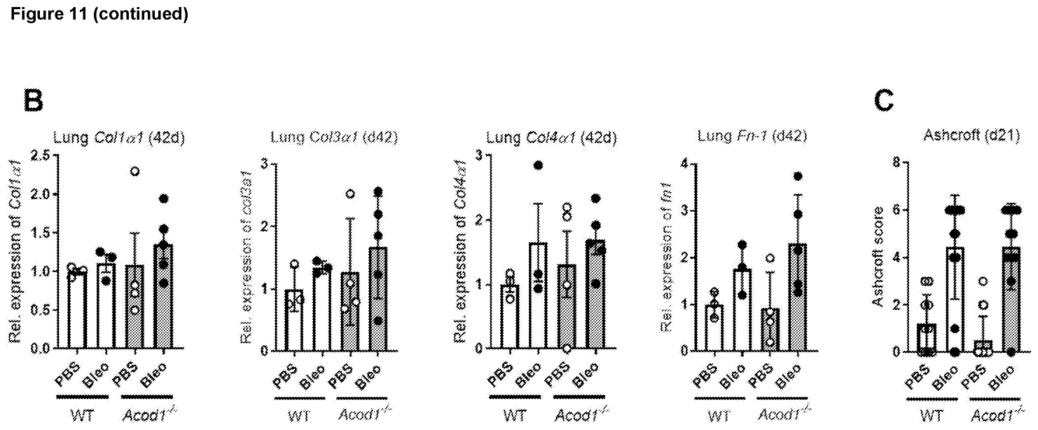

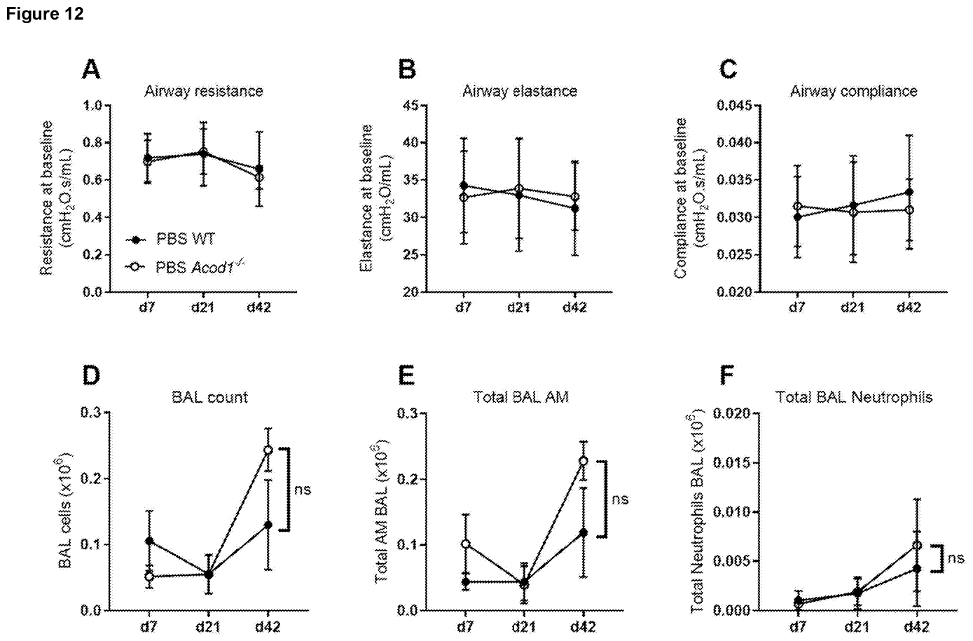

To mechanistically assess the role of Acodl/itaconate in the pathogenesis of pulmonary fibrosis, the murine bleomycin model of pulmonary fibrosis was utilised. Wild-type (WT) mice were instilled with a single dose of bleomycin via the oropharyngeal route (FIG. 1C) and expression of Acod1 in lungs assessed at inflammatory (d7), peak fibrosis (d21) and late (d42) phases of the disease. Compared to PBS controls, Acod1 expression was significantly elevated at d7 and d21 post bleomycin, returning to baseline levels at d42. Acod1 reached maximum-expression levels at d21 post bleomycin, corresponding with both inflammation and peak fibrosis (FIG. 1D). Furthermore, itaconate levels in BAL were assessed by targeted gas chromatography-mass spectrometry (GC-MS) at these different time points. Compared to PBS controls, itaconate was significantly increased at d7 and d21 post bleomycin and returned to baseline levels at d42 (FIG. 1E). In order to determine whether Acod1 played a role in the establishment or severity of fibrosis, the response of Acod1-deficient mice to bleomycin was assessed. Although WT mice showed improved fibrosis, pathology and lung function, Acod1^{-/-} mice failed to return to baseline levels at d42 and had increased BAL cell counts (FIG. 1H). In order to determine the impact of Acod1-deficiency on immune responses in the lung, multi-parameter flow cytometry using a gating strategy shown in FIG. 9A was performed. Although total numbers of AMs are much higher post bleomycin than numbers of neutrophils, both were elevated in Acod1^{-/-} compared to WT mice at d42 (FIG. 1F and G). Furthermore, Acod1^{-/-} had worsened airway resistance, elastance and compliance compared to WT controls at day 42 post bleomycin exposure (FIG. 11). However, in comparison to WT controls, Acod1-deficient mice did not show altered lung function (FIG. 10A and C), total BAL counts (FIG. 10B and D) or pathology (FIG. 11C) at d7 or d21 post-injury, suggesting that itaconate does not play a role in the initiation of fibrosis. Total AM numbers (FIG. 10E) were not altered when comparing WT and Acod1 -1 mice at d7 or d21 post challenge. A reduction in neutrophil numbers was observed, comparing WT and Acod1^{-/-} mice, at day 7 post challenge, but no alteration at d21 (FIG. 10F). Adaptive immunity did not appear to be altered in Acod1^{-/-} mice compared to WT controls, with no alteration in T- or NK-cells at any time-point (FIGS. 10G and H). Acod1^{-/-} mice showed increased expression of Collagen-(Col)360 1 and Fibronectin-1 (Fn-1) (FIG. 2A), compared to WT controls at day 21, but not at d7/42 (FIG. 11A and B). No statistically significant change in lung expression of Col1α1 or Col4α1 was observed at any time point assessed in this model (FIG. 2A and FIG. 11A-B). Ashcroft scoring of Sirius red stained lung slices indicated that pathology did not change in Acod1^{-/-} mice at day 21 post bleomycin compared to WT (FIG. 11C). Acod1^{-/-} ^{-/-} mice had enhanced pulmonary fibrosis compared to WT controls, characterized by increased lung hydroxyproline levels (FIG. 2B) and Ashcroft scores (FIG. 2C and D). Consistent with these findings, Acod1^{-/-} mice showed increased levels of superoxide in in the CD45⁺ compartment of whole lung tissue in comparison to WT (FIG. 2E, FIG. 9B), further suggesting more severe disease in Acod1^{-/-}. Furthermore, no change in lung function parameters, BAL cell count, AMs or neutrophils was detected at baseline (in PBS mice) in WT compared to Acod1^{-/-} mice (FIG. 12). Collectively, these data suggest that Acod1-deficiency results in more severe pulmonary fibrosis in response to inhaled bleomycin, in comparison to WT controls.

### Example 3: Itaconate Controls Tissue Resident AM Metabolism

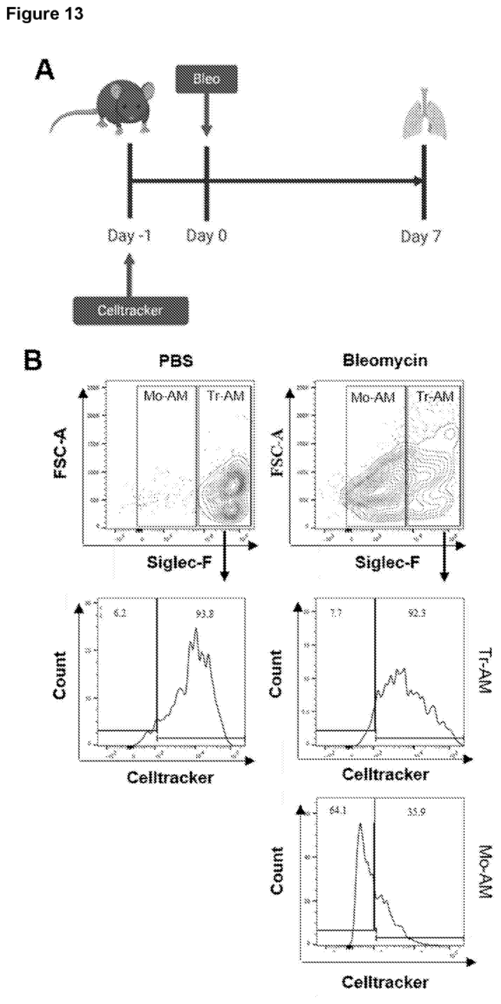

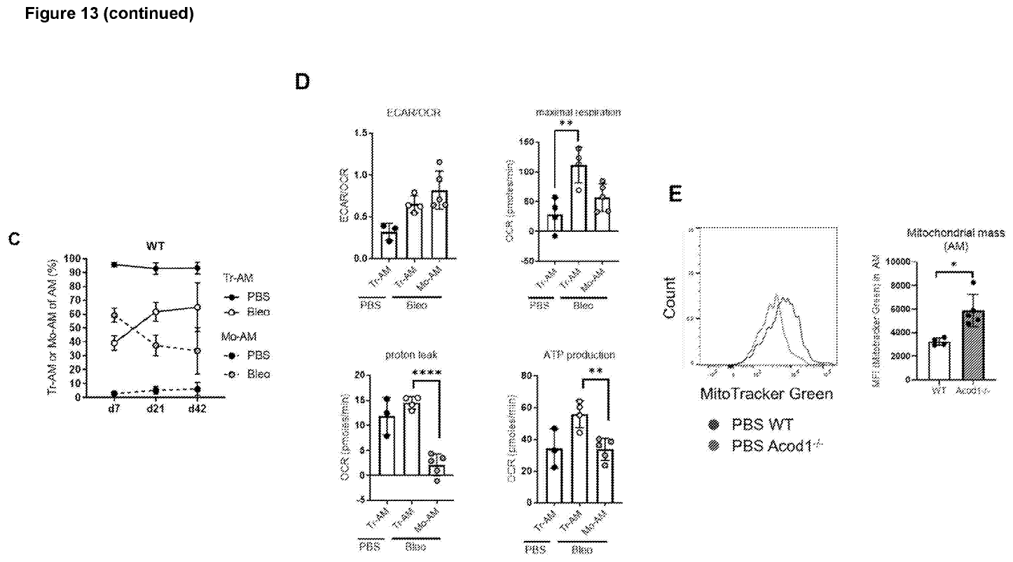

Recruited, monocyte-derived AMs (Mo-AMs), as opposed to fetally derived tissue resident AMs (Tr-AMs), have been shown to drive the pathogenesis of pulmonary fibrosis in mice. However, whether these ontologically discrete cell types are metabolically distinct is not known in the art. In the bleomycin model, recruited Mo-AMs may be identified on the basis of Siglec-F expression: specifically, Tr-AMs were Siglec-F^{hi}, whereas Mo-AMs were Siglec-F^{int} (Misharin et al., 2017, J. Exp. Med., 214(8):2387-2404). These findings were confirmed by first labelling the lung with a cell permeable die (cell-tracker), prior to the administration of bleomycin (FIG. 13A). Consistent with the published findings, the bulk of Tr-AMs were labelled, whereas the majority of Mo-AMs were unlabelled, indicating recent recruitment (FIG. 13B). In the present murine model, proportions of Tr-AMs were diminished at d7 after bleomycin exposure in WT mice, while Mo-AMs made up a high proportion of AMs at d7, reducing by d21 and d42 (FIG. 13C). The highest number of Mo-AMs were observed at d7 post bleomycin and fibrogenic changes occur in the bleomycin mouse model from day 8 onwards (Moeller et al., 2008 al., 2008, Int. J. Biochem. Cell Biol., 40(3):362-82). Consequently, the metabolic activity of Mo-AMs and Tr-AMs sorted from bleomycin exposed mice during this early stage of fibrosis development (d7) was assessed next. In WT mice, Tr-AMs became highly oxidative after bleomycin exposure, whereas Mo-AMs had comparatively diminished baseline oxygen consumption rate (OCR,

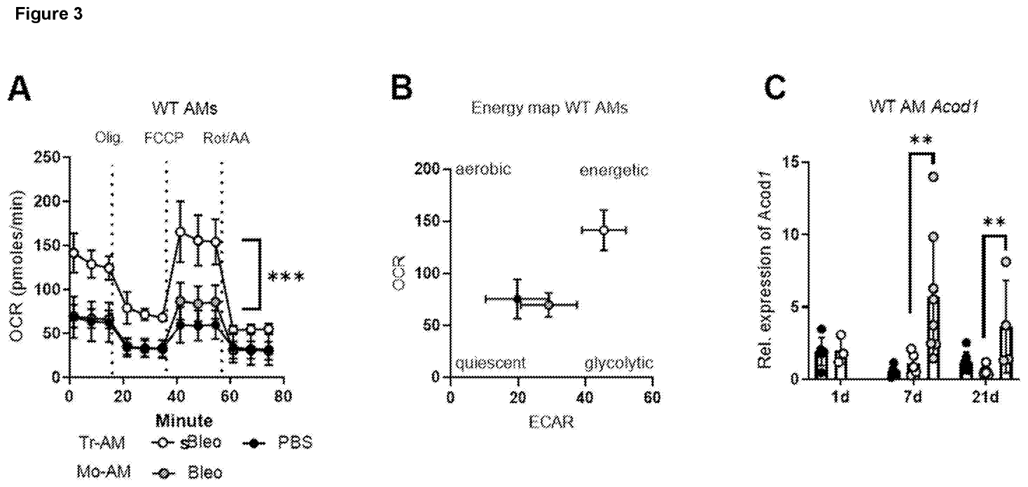

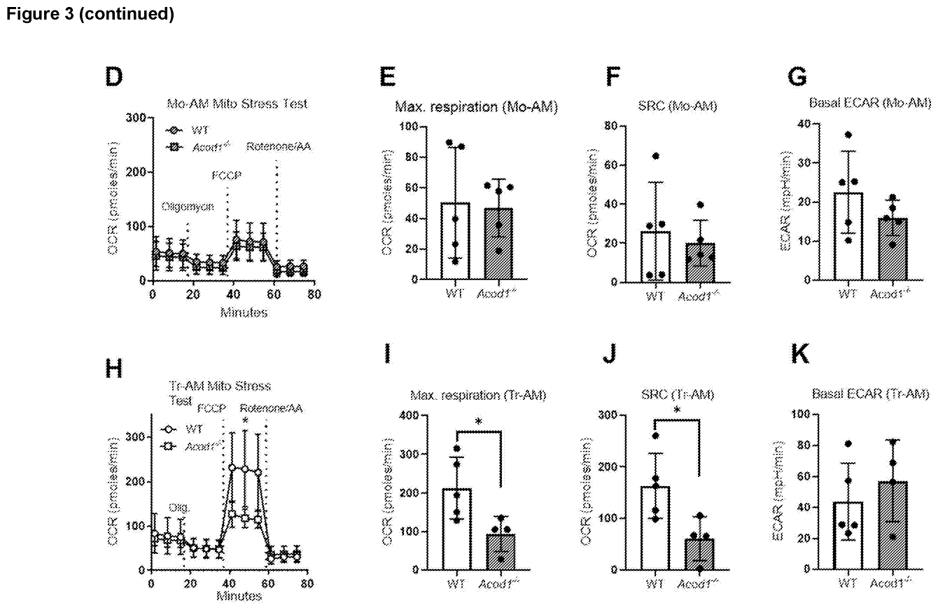

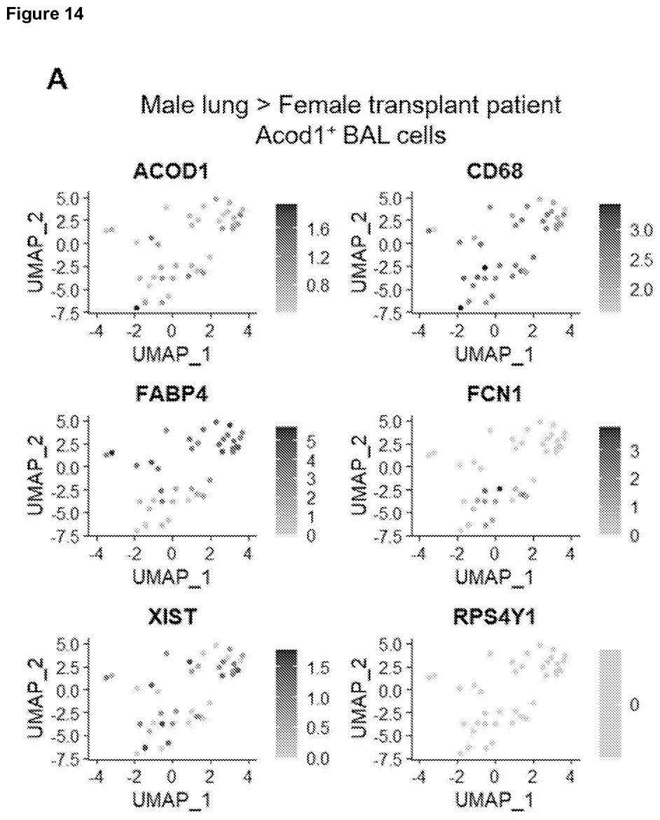

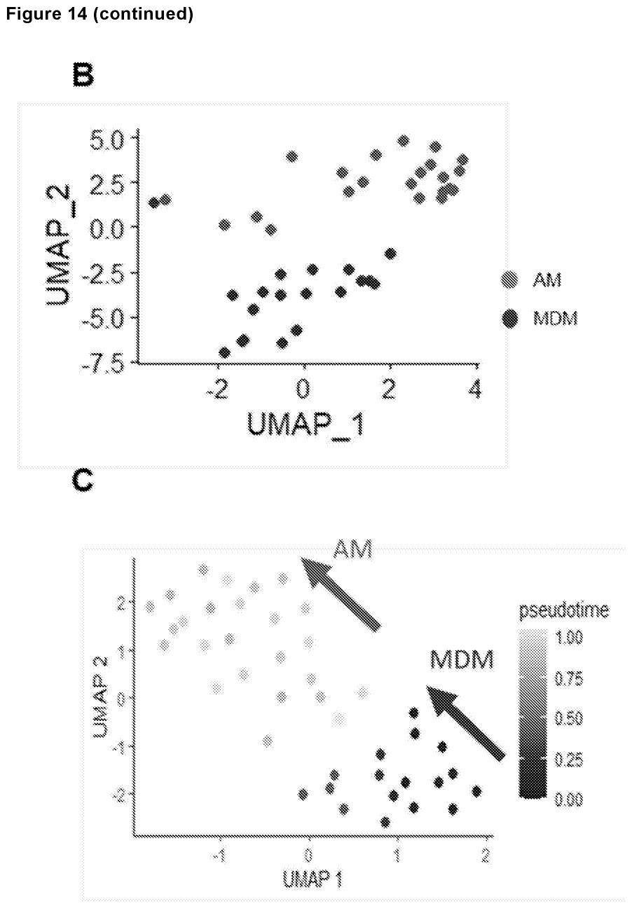

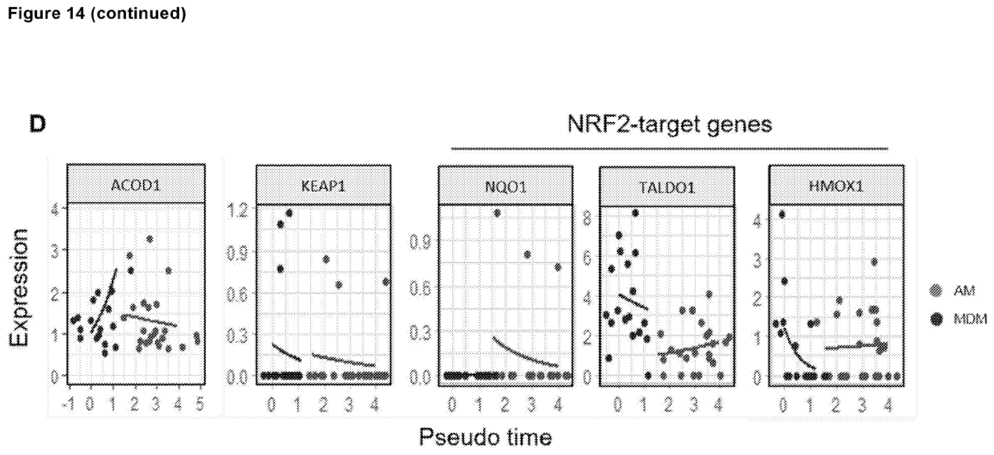

FIG. 3A and B), showing that Mo-AM and Tr-AM are metabolically distinct (FIG. 13D). Importantly, sorted Mo-AMs highly expressed Acodl, in comparison to Tr-AMs from control or bleomycin mice (FIG. 3C). Since itaconate has recently been shown to control macrophage metabolism and effector functions in vitro (Lampropoulou et al., 2016, Cell Metabolism, 24:158-166), the metabolic impact of CAD deficiency on AM subtypes was assessed. Using the Seahorse Mito Stress Test, oxygen consumption rate (OCR) and extracellular acidification rate (SCAR) were measured at baseline and after the sequential addition of 1.5 μM Oligomycin, 2.0 μM FCCP and 0.5 μM Rotenone/Antimycin A. WT Mo-AMs had similar levels of oxidative phosphorylation (OxPhos) and glycolysis (SCAR) compared to Acod1^{-/-} cells (FIG. 3D-G). However, Acod1^{-/-} Tr-AMs had reduced OCR (FIG. 3H), maximal respiration (FIG. 31) and spare respiratory capacity (SRC, FIG. 3J) in comparison to WT Tr-AMs, while basal SCAR remained unchanged (FIG. 3K). Together these results indicate that during lung fibrosis in mice, recruited Mo-AMs are characterized by a quiescent metabolic phenotype and in contrast, resident Tr-AMs are highly oxidative. Furthermore, CAD expression is a critical regulator of metabolism in tissue resident AMs during lung fibrosis, as itaconate deficiency leads to decreased oxidative phosphorylation. Recently we used single cell sequencing of BAL samples from sex-mismatched lung transplant patients to identify recruited AMs in the human airways (Byrne et al., 2020, J Exp. Med., 217(3)). Analysis of ACOD-expressing cells in a male donor to female recipient, showed a subset of ACOD1 expressing AMs, which are monocyte derived (MDMs; FIG. 14A-B, as they do not express the male cell identifier RPS4Y1 but do express the female identifier gene XIST). Consistent with recent work showing itaconate as a regulator of oxidative stress (Mills et al., 2018, Nature, 556:113-117), pseudo time analysis showed that ACOD1 increases as MDMs differentiate to mature AMs, while expression of NRF2-target gene HMOX1 decreases (FIG. 14C-D).

### Example 4: Acod1 Deficient Tissue Resident AMs Are More Pro-Fibrotic Post Bleomycin

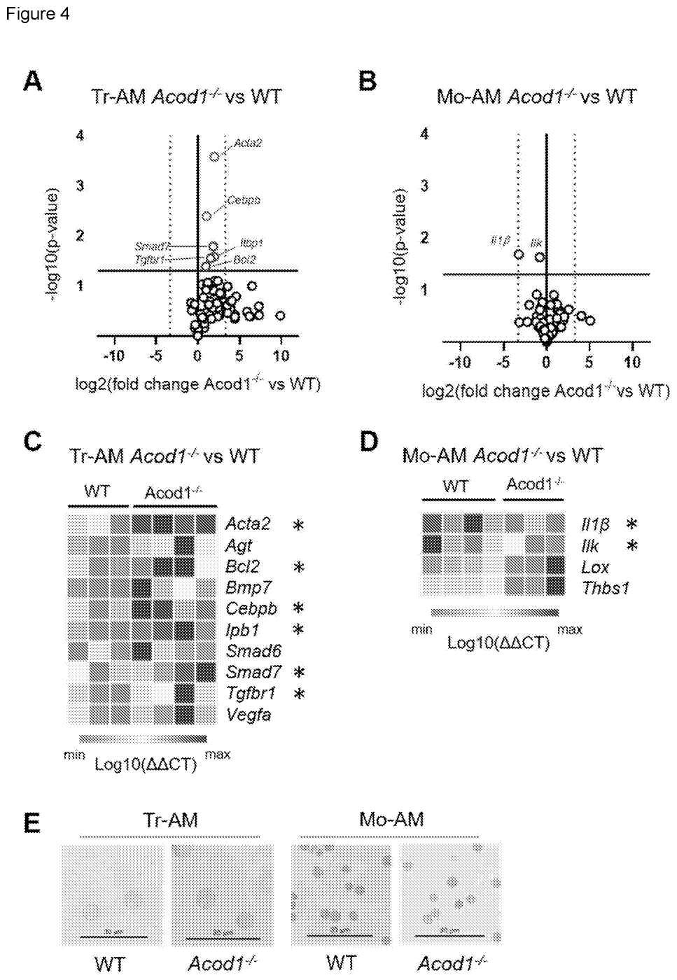

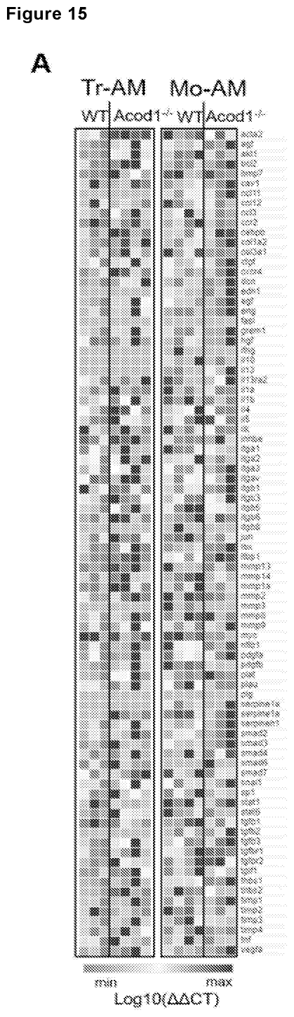

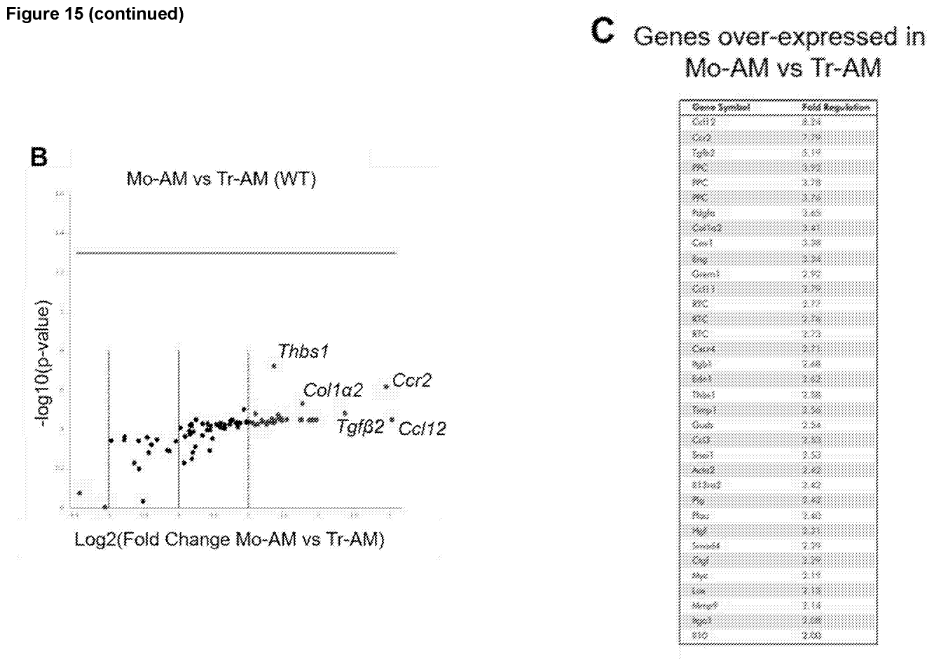

How itaconate influenced fibrotic pathways in Mo-AMs and Tr-AMs, sorted from BAL at day 7 post bleomycin challenge, was next assessed. In order to determine whether fibrotic pathways in AMs were impacted by itaconate deficiency, a PCR array that interrogates 84 genes involved in the fibrosis cascade was used (FIG. 15A). Consistent with previous findings (Misharin et al., 2017, J. Exp. Med., 214(8):2387-2404), Tr-AMs and Mo-AMs differed in their response to bleomycin-induced lung fibrosis, with Mo-AMs comparatively high expressers of genes implicated in fibrotic signalling processes such as Col1α2, Transforming growth factor β2 (Tgfβ2) and Ccr2 (FIG. 15A and B). Comparing Acod1^{-/-} and WT sorted airway macrophages, the data indicate that itaconate deficiency significantly increased gene expression of fibrosis related genes in Tr-AMs (FIG. 4A), while it downregulated the expression of only two genes in Mo-AMs (FIG. 4B). In FIG. 4C and D the annotated genes are those that showed significant change in expression or at least 10-fold increase in Acod1^{-/-} AMs, compared to WT cells. In Mo-AMs, IL-1β, and Integrin linked kinase (Ilk) were significantly decreased in Acod1^{-/-} compared to WT cells, with no significant upregulation in any pro-fibrotic factors (FIG. 4C). However, in Tr-AMs, pro-fibrotic mediators including CCAAT enhancer binding protein β (Cebpb), Tgfβr1 and Smad7 were significantly increased in Acod1^{-/-} Tr-AMs compared to WT cells (FIG. 4D). Examination of cellular morphology (cytospins after Diff-Quik staining) showed that while itaconate-deficient Tr-AMs and Mo-AMs are metabolically and transcriptionally distinct in Acod1^{-/-} mice, their size and granularity is unchanged (FIG. 4E). Together, these data show that itaconate regulates pro-fibrotic pathways in Tr-AMs but not in Mo-AMs.

### Example 5: Adoptive Transfer of WT, But Not Acod1^{-/-} Mo-AMs, Into the Airways of Acod1^{-/-} Bleomycin Treated Mice Rescued the Fibrotic Phenotype

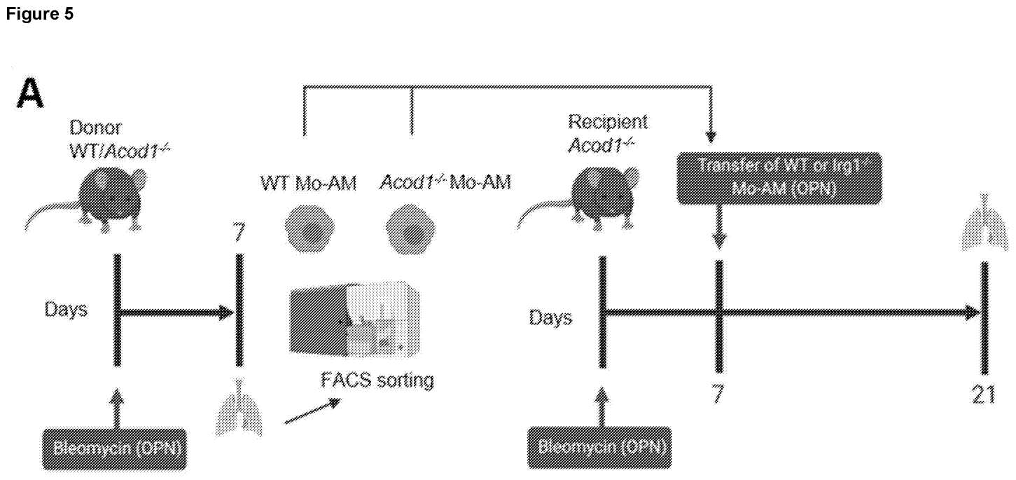

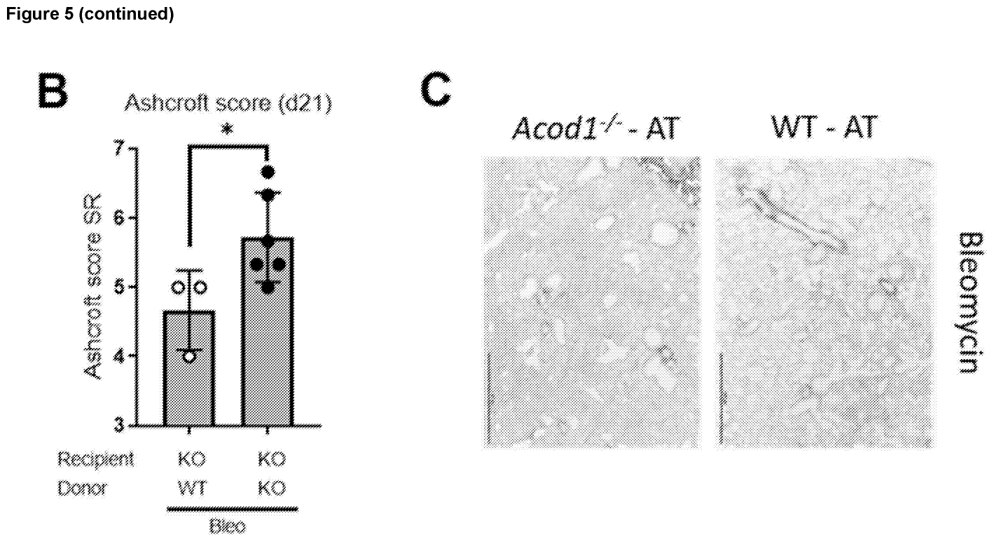

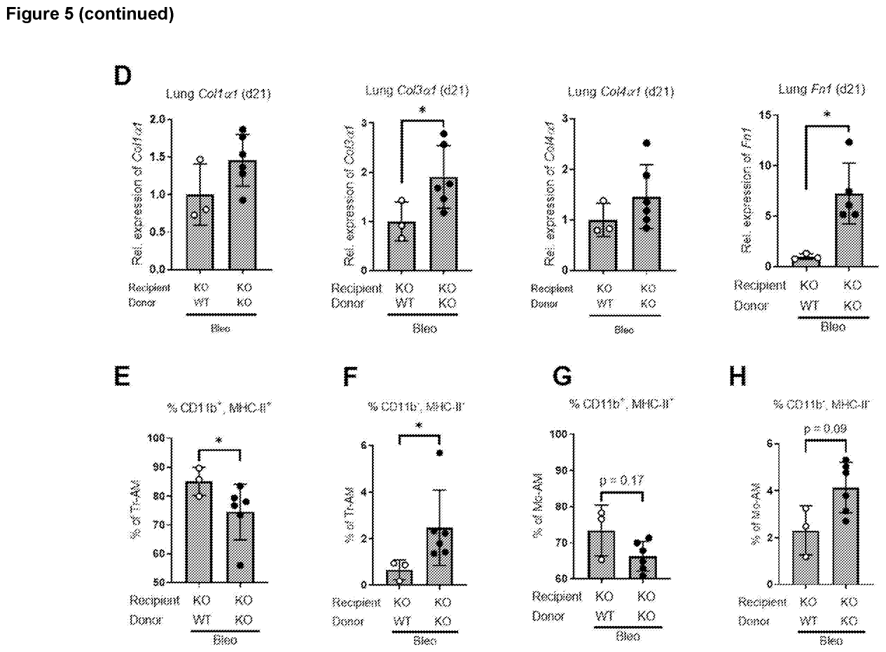

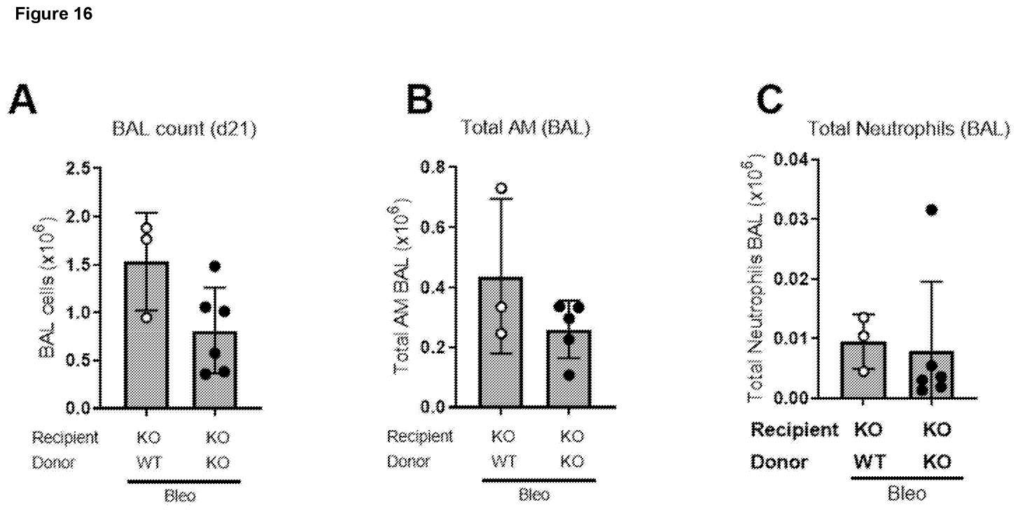

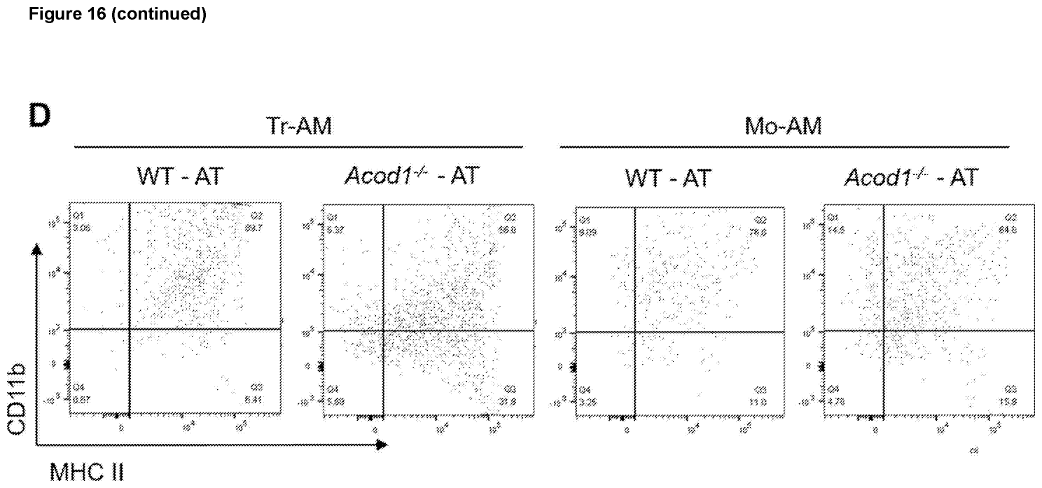

As Mo-AMs highly express Acod1 in comparison to TR-AMs, it was investigated whether transfer of WT or Acod1^{-/-} Mo-AMs into an Acod1^{-/-} fibrotic environment had differential effects on the course of disease. Sorted Mo-AMs from WT or Acod1^{-/-} mice were adoptively transferred into the airways of Acod1^{-/-} mice at day 7 post bleomycin (FIG. 5A). Interestingly, transfer of WT, but not Acod1^{-/-} Mo-AMs, into the airways of Acod1^{-/-} bleomycin treated mice rescued the fibrotic phenotype, characterized by decreased Ashcroft scores based on Sirius red staining (FIG. 5B and C) and decreased gene expression of lung Col3α1 and Fn1 (FIG. 5D). However, BAL cell count, total AM and neutrophil numbers as well gene expression of Col160 1 and Col4α1 remained unchanged after adoptive transfer (FIG. 5D and FIG. 16). Next, we assessed whether AM phenotype could be altered by adoptive transfer of WT Mo-AMs in to Acod1^{-/-} mice. Expression of macrophage activation markers CD11b and MHC II (Byrne et al., 2017, Mucosal Immunology, 10:716-726; Krausgruber et al., 2011, Nat. Immunology, 12(3):231-8) were assessed in Mo-AMs and Tr-AMs at day 21 post bleomycin with adoptive transfer of WT or Acod1^{-/-} Mo-AMs (FIG. 16D). While Tr-AMs showed an increased proportion of activated cells upon adoptive transfer of WT Mo-AMs into Acod1^{-/-} (FIG. 5E) and a decreased proportion of CD11b⁻/MHCII⁻ cells (FIG. 5F), this trend was not significantly altered in Mo-AMs (FIG. 5G-H). Collectively, these results suggest that adoptive transfer of itaconate-sufficient Mo-AMs rescues disease phenotype induced by bleomycin exposure in Acod1^{-/-} mice and alters Tr-AM phenotype.

### Example 6: Exogenous Itaconate Limits Human Lung Fibroblast Wound Healing

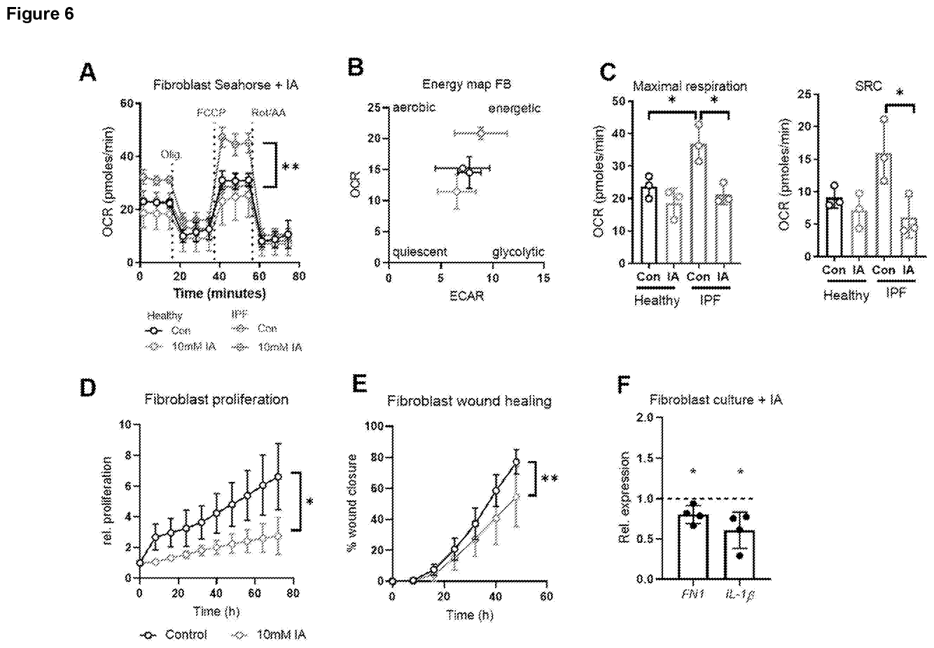

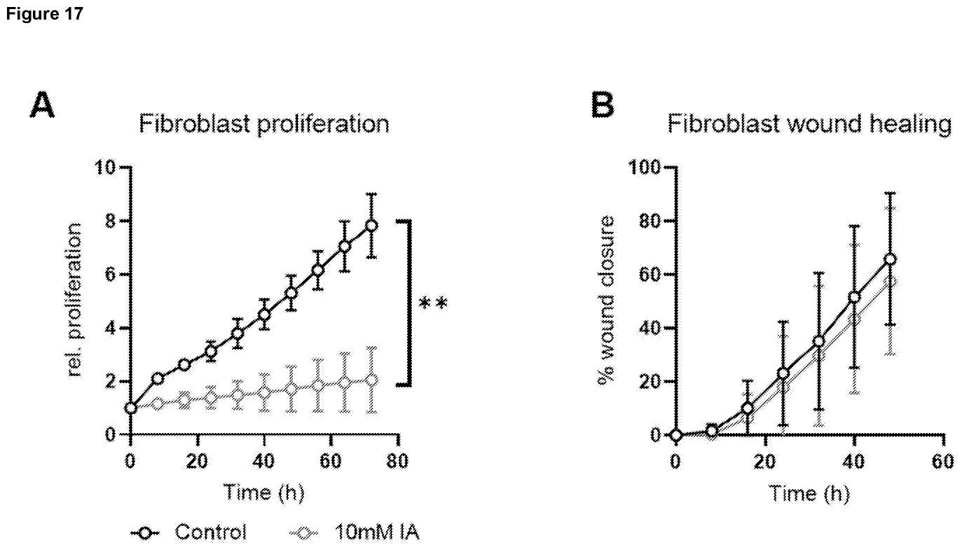

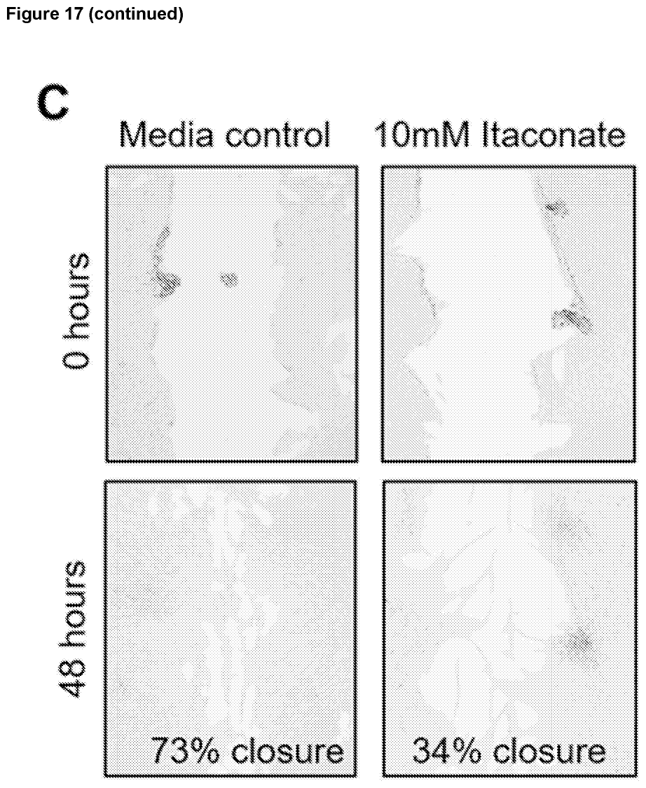

Fibroblasts are the principle effector cell during lung fibrosis and the main source of the excessive extracellular matrix deposition seen during the disease (Kendall and Feghali-Bostwick, 2014, Front. Pharmacology, 5:1-13), however our data indicate that these cells do not express Acod1 (data not shown). Since macrophages are known to regulate the pro-fibrotic activity of fibroblasts in the lung (Byrne et al., 2016, Trends Mol. Med. 22:303-316) and itaconate is secreted into the airways (FIGS. 1B and 1E), it was next assessed whether itaconate could directly influence fibrosis by limiting the metabolic and pro-fibrotic activity of human lung fibroblasts (HLF). Human lung fibroblasts were cultured in media alone or supplemented with itaconate and assessed after 24 h-72 h. After 24 h incubation we assessed OCR in response to Oligomycin, FCCP and rotenone/Antimycin A (FIG. 6A) as well as baseline ECAR (FIG. 6B) and found that IPF HLFs have increased maximal respiration and spare respiratory capacity compared to healthy HLFs and this effect was ameliorated after stimulation with itaconate (FIG. 6C). To assess the ability of itaconate to limit fibrosis related functions of HLFs, proliferation and wound healing assays were carried out in the presence or absence of itaconate. After exposure to itaconate, HLFs showed significantly reduced proliferative capacity over 72 h in both cells derived from healthy donors (FIG. 6D) as well IPF patients (FIG. 17A), while the ability to close a standardized wound over a 48 h period was decreased in healthy HLFs (FIG. 6E) but not in IPF fibroblasts (FIG. 17B and C). Furthermore, culture with itaconate downregulated the gene expression of IL-1β and FN-1 in healthy HLFs (FIG. 5F). Taken together these data suggest that itaconate impacts fibroblast metabolic phenotype, proliferation and wound healing thereby limiting the severity of pulmonary fibrosis.

### Example 7: Inhaled Ataconate is Anti-Fibrotic

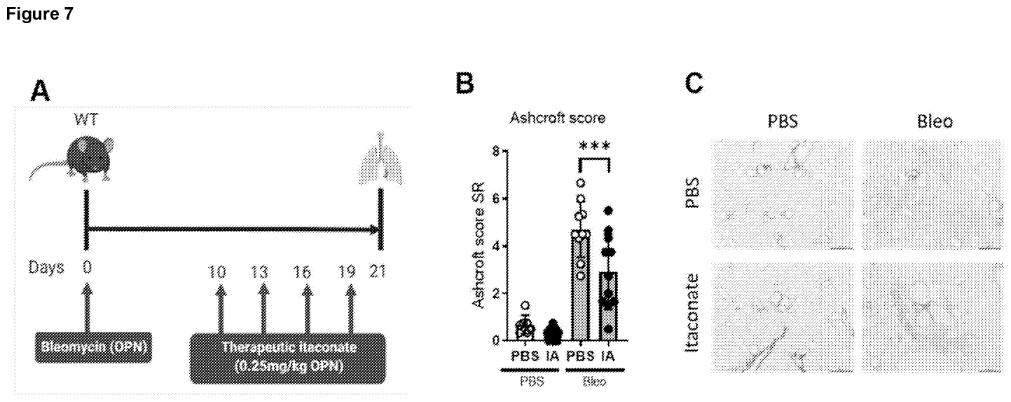

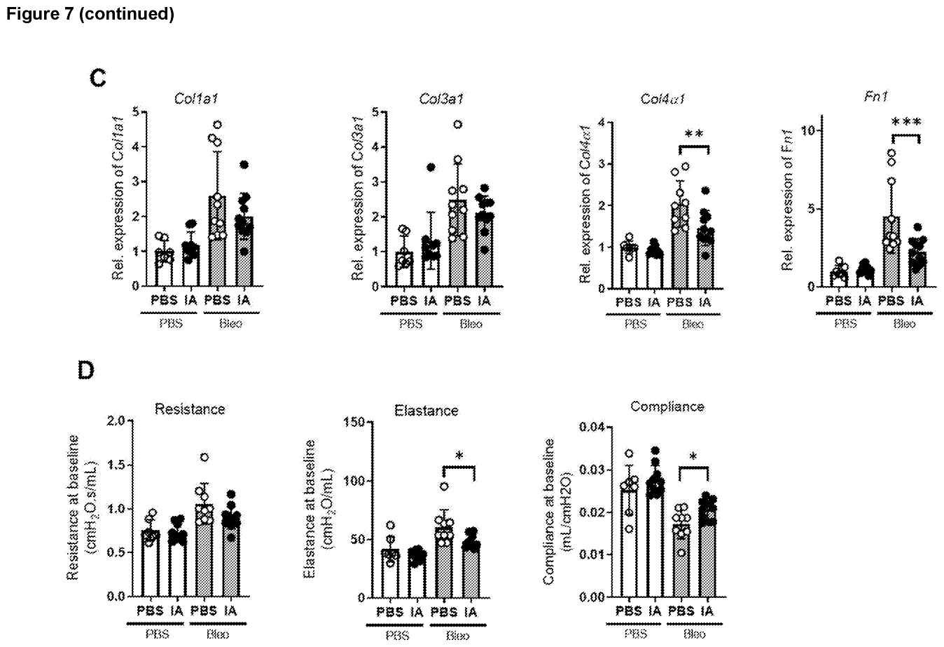

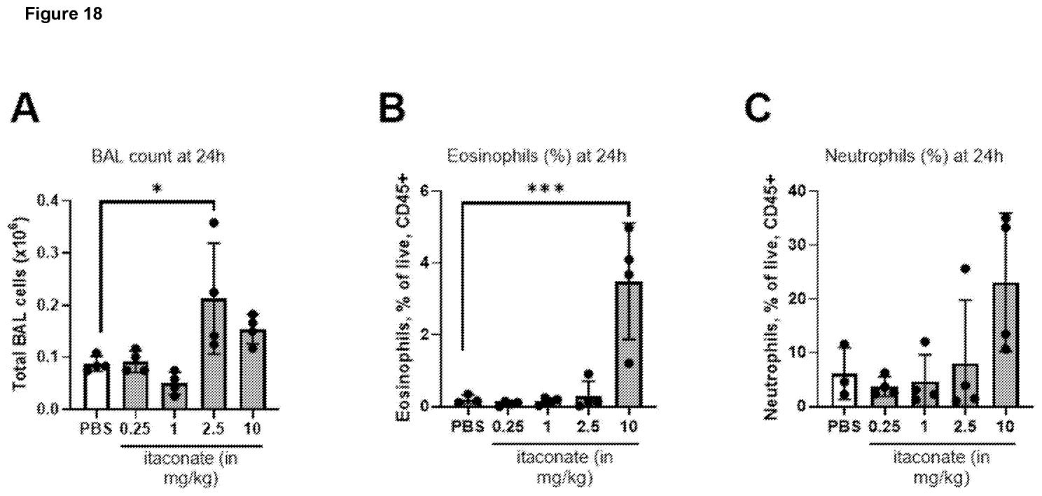

The data herein show that ACOD1 expression is reduced in IPF AMs (FIG. 1), that Acod1^{-/-} mice have worsened pulmonary fibrosis in comparison to controls (FIGS. 1 and 2), and that itaconate can limit fibroblast wound healing capacities (FIG. 6). These data raise the intriguing possibility that exogenous itaconate could improve severity of lung fibrosis. In order to address whether inhaled itaconate is anti-fibrotic, it was first determined a dose of itaconate that would not provoke an inflammatory response in murine airways. It was found that an inhaled dose of 0.25 mg/kg was well tolerated after single (FIG. 18A-C) and/or repeated (data not shown) oropharyngeal (OPN) administration in naïve mice and subsequent experiments were carried out at this dose. We administered inhaled (OPN) itaconate or PBS twice a week for two weeks during the fibrotic phase (starting at day 10 post bleomycin) to WT mice (FIG. 7A); this dosing strategy is in accordance with the American Thoracic guidelines for preclinical assessment of potential therapies for IPF (Jenkins et al., 2017). Subsequently pathology, fibrosis and lung function at d21 post bleomycin were assessed. Remarkably, inhaled itaconate significantly ameliorated all major hallmarks of lung fibrosis, including Ashcroft score based on Sirius red staining (FIG. 7B-C) expression of Col4α1 and Fn1 (FIG. 7C) and lung airway elastance and compliance (FIG. 7D). Taken together these results demonstrate that inhaled itaconate significantly improves bleomycin induced pulmonary fibrosis.

### Discussion

The data presented herein identified a critical role for the Acod1/itaconate pathway in the pathogenesis of pulmonary fibrosis. These data demonstrate that the ACOD1/itaconate pathway is significantly disrupted in IPF and that Acod1^{-/-} mice have more severe lung disease in a murine model of pulmonary fibrosis. Acod1 influences fibrotic responses in AMs as Acod1^{-/-} AMs demonstrated impaired metabolism and enhanced expression of pro-fibrotic genes, while adoptive transfer of WT monocyte-recruited AMs into the lungs of Acod1^{-/-} improved bleomycin induced pulmonary fibrosis and altered Tr-AM phenotype. Ex vivo culture of human fibroblasts with itaconate reversed their metabolic reprogramming in IPF and decreased both proliferation and wound healing capacity. The data also demonstrate that therapeutic administration of inhaled itaconate in vivo ameliorates bleomycin-induced pulmonary fibrosis in mice. Thus, the work presented herein indicates that the ACOD1/itaconate axis as a novel, endogenous anti-fibrotic pathway which is dysregulated during IPF. The data highlight therapeutic potential of promoting the ACOD1/itaconate pathway and in particular pharmacological approaches which deliver itaconate or its derivatives as anti-fibrotic agents.

It is now well established that in mice, lung-resident AMs maintain their populations via proliferation in situ during homeostasis and that a second population of ontologically distinct Mo-AMs are recruited from peripheral monocytes during ongoing inflammatory responses. These recruited Mo-AMs, rather than fetally derived tr-AMs are essential for the development of pulmonary fibrosis in murine models, whereas deletion of tissue-resident AMs had no effect on the disease. The data herein indicate that Acod1 is differentially expressed in Tr-AMs and Mo-AMs and that these cell types are metabolically distinct.

AMs play an important role in defending the lung environment from inhaled threats and are key sentinels of pulmonary homeostasis. AM phenotype is a critical component of lung immunity and manipulation of AM phenotype can have drastic consequences for lung health. Itaconate has emerged as a key autocrine immunoregulatory component involved in activation of bone-marrow derived macrophages (BM DMs), however to-date little was known regarding the specific role of itaconate in highly specialized tissue resident macrophage populations during chronic disease, such as those found in the airways in IPF. Itaconate is a critical component of pulmonary responses to MTb infection as both global Acod1^{-/-} and myeloid-specific Acod1^{-/-} knockouts rapidly succumb to infection. Collectively, these data outline a potential role for the ACOD1/itaconate axis in the lung during infection. However, until the present investigates, the role of the ACOD1/itaconate axis in tissue fibrosis has never been explored. The data herein in the context of pulmonary fibrosis highlight itaconate as a critical component of respiratory immunity and tissue immunity

While we show that in the bleomycin mouse model, itaconate is increased in the BAL during the inflammatory stage and recovers to baseline levels during the late phase, in human AMs ACOD1 is highly expressed at homeostasis and disrupted during pulmonary fibrosis. This is particularly pertinent as there are a dearth of data regarding the role of itaconate during human disease and as a predisposition towards development of chronic lung disease. It is shown herein for the first time that itaconate can directly influence human lung fibroblast phenotype and function in vitro, which is potentially part of the mechanism behind the improved lung function, collagen gene expression and deposition observed upon administration of inhaled itaconate during the fibrotic phase of the bleomycin mouse model. These findings indicate that itaconate potentially mediates paracrine effects on other stromal or immune cells types as it is actively secreted at homeostasis and thus may have implications for chronic diseases of the lung or other tissues, in which fibrosis plays a role.

In conclusion, this work defines a novel regulatory pathway, which is impaired during fibrotic disease, particularly fibrotic lung disease. This novel relationships between ACOD1, airway macrophages and fibrosis have the potential to impact therapies for IPF and highlight ACOD1, itaconate or its metabolites as molecular targets for the treatment of fibrotic diseases.

1. A method of treating or preventing tissue fibrosis, said method comprising administering:
a) an agent which inhibits succinate dehydrogenase; or
b) a population of monocyte-recruited macrophages (Mo-Ms);
to a subject in need thereof.

2. The method according to claim 1, wherein:
a) the agent directly inhibits succinate dehydrogenase, wherein optionally the agent is selected from a small molecule, a nucleic acid, an antibody or antigen-binding fragment thereof, or an aptamer; and/or
b) the agent indirectly inhibits succinate dehydrogenase, wherein optionally the agent increases the expression and/or activity of aconitate decarboxylase 1 (ACOD1), wherein optionally the agent is a nucleic acid, a protein, or a small molecule,.

3. The method according to claim 2, wherein the small molecule compound is itaconate or a derivative or analogue thereof, optionally in the form of a pharmaceutically acceptable salt.

4. The method according to claim 1, wherein the agent is administered by: inhalation; intraperitoneal, subcutaneous, and/or intramuscular injection; infusion; and/or orally, preferably wherein the agent is administered by oropharyngeal inhalation and/or nasal inhalation.

5. The method according to claim 1, wherein the agent is delivered in a drug delivery system, wherein optionally said drug delivery system:
a) specifically targets phagocytes; and/or
b) is a liposome-based drug delivery system.

6. The method according to claim 3, wherein the itaconate, derivative, analogue or pharmaceutically acceptable salt thereof is administered at a dose of about 0.1 mg/kg to about 10 mg/kg.

7. The method according to claim 3, wherein the itaconate, derivative, analogue or pharmaceutically acceptable salt thereof is administered once per week to about four times per day, preferably about once per day.

8. (canceled)

9. The method according to claim 1, wherein the Mo-M express Acod1, and optionally have a quiescent metabolic phenotype.

10. The method according to claim 1, wherein:
a) the population of Mo-Ms is administered directly to an individual to be treated; or
b) the population of Mo-Ms is recruited following administration of a composition which stimulates targeting of Mo-Ms to a tissue to be treated.

11. The method according to claim 1, wherein the Mo-M are:
a) autologous Mo-Ms; or
b) allogenic Mo-Ms.

12. The method according to claim 1, wherein the treatment or prevention modifies the metabolic and/or fibrotic phenotype of tissue-resident macrophages (Tr-Ms), preferably wherein the treatment or prevention increases the metabolic phenotype and/or reduces the fibrotic phenotype of the Tr-M.

13. The method according to claim 12, wherein the treatment or prevention increases the proportion of CD11b⁺/MHCII⁺ Tr-Ms resident in the tissue.

14. The method according to claim 12, wherein the treatment or prevention modifies the metabolic and/or fibrotic phenotype of fibroblasts within the tissue, preferably wherein the treatment or prevention reduces the metabolic and/or fibrotic phenotype of the fibroblasts.

15. The method according to claim 14, wherein the treatment or prevention:
a) reduces oxygen consumption rate, maximal respiration and/or spare respiratory capacity of fibroblasts;
b) reduces proliferation of fibroblasts; and/or
c) reduces the wound healing capacity of fibroblasts.

16. The method according to claim 1, wherein the treatment or prevention results in:
a) an improvement in the fibrosis of the tissue;
b) a decrease in tissue collagen expression, preferably Col3α1, Col1α1 and/or Col4α1;
c) a decrease in tissue fibronectin (Fn1) expression;
d) a decrease in IL-1β expression in fibroblasts obtained from the tissue; and/or e) a decrease in hydroxyproline levels.

17. The method according to claim 1, wherein the fibrosis is pulmonary fibrosis, liver fibrosis, kidney fibrosis, intestinal fibrosis, cardiac fibrosis, myelofibrosis and/or skin fibrosis.

18. The method according to claim 17, wherein the pulmonary fibrosis is any form of chronic fibrosing interstitial lung disease including idiopathic pulmonary fibrosis.

19. The method according to claim 18, wherein the treatment or prevention results in:
a) an improvement in lung function, preferably an increase in forced vital capacity, an increase in total lung capacity and/or an increase in the transfer capacity of the lung for the uptake of carbon monoxide, as measured by gas transfer (TLco) test;
b) a reduction in the decline of forced vital capacity;
c) preservation or improvement of exercise capacity;
d) a reduction in the progression of fibrosis as quantified by high resolution computed tomography;
e) preservation or improvement of quality of life; and/or
(f) improved survival.

20. The method according to claim 1, wherein the subject to be treated has reduced levels of itaconate in a sample of the tissue to be treated, and wherein the tissue fibrosis is pulmonary fibrosis, the sample is optionally a bronchoalveolar lavage (BAL) sample.

21. The method according to claim 1, wherein the subject to be treated has Tr-Ms with reduced ACOD1 expression.

22. The method according to claim 1, wherein the agent or population of Mo-Ms is for use in combination with another therapeutic.

23. A method for identifying a compound which inhibits fibrosis progression, comprising the steps of:
a) culturing cells in vitro;
b) adding a test compound to the cultured cells; and
c) determining a change in the metabolic phenotype of the cells in response to the test compound;
wherein the change in metabolic phenotype of the cells is a reduction or increase in the metabolic phenotype of the cells; wherein preferably the cells are fibroblasts or Tr-Ms.

24. The method according to claim 23, wherein:
a) a reduction or increase in the metabolic phenotype of the cells is:
i) a reduction or increase in the oxygen consumption rate of the cells;
ii) a reduction or increase in the maximal respiration of the cells; and/or
iii) a reduction or increase in the spare respiratory capacity of the cells; and/or
(b) the method further comprises a step of determining a reduction in the fibrotic phenotype of the cells in response to the test compound.

25. (canceled)


SEQUENCE LISTING (10 sequences)

SEQ ID NO: 1 - protein, 664 aa, Homo sapiens
MSGVRGLSRLLSARRLALAKAWPTVLQTGTRGFHFTVDGNKRASAKVSDSISAQYPVVDHEFDAVVVGAGGAGLRAAFGLSEAGFNTACVTKLFPTRSHTVAAQGGINAALGNMEEDNWRWHFYDTVKGSDWLGDQDAIHYMTEQAPAAVVELENYGMPFSRTEDGKIYQRAFGGQSLKFGKGGQAHRCCCVADRTGHSLLHTLYGRSLRYDTSYFVEYFALDLLMENGECRGVIALCIEDGSIHRIRAKNTVVATGGYGRTYFSCTSAHTSTGDGTAMITRAGLPCQDLEFVQFHPTGIYGAGCLITEGCRGEGGILINSQGERFMERYAPVAKDLASRDVVSRSMTLEIREGRGCGPEKDHVYLQLHHLPPEQLATRLPGISETAMIFAGVDVTKEPIPVLPTVHYNMGGIPTNYKGQVLRHVNGQDQIVPGLYACGEAACASVHGANRLGANSLLDLVVFGRACALSIEESCRPGDKVPPIKPNAGEESVMNLDKLRFADGSIRTSELRLSMQKSMQNHAAVFRVGSVLQEGCGKISKLYGDLKHLKTFDRGMVWNTDLVETLELQNLMLCALQTIYGAEARKESRGAHAREDYKVRIDEYDYSKPIQGQQKKPFEEHWRKHTLSYVDVGTGKVTLEYRPVIDKTLNEADCATVPPAIRSY

SEQ ID NO: 2 - DNA, 2260 bp, Homo sapiens
gcagacatgtcgggggtccggggcctgtcgcggctgctgagcgctcggcgcctggcgctggccaaggcgtggccaacagtgttgcaaacaggaacccgaggttttcacttcactgttgatgggaacaagagggcatctgctaaagtttcagattccatttctgctcagtatccagtagtggatcatgaatttgatgcagtggtggtaggcgctggaggggcaggcttgcgagctgcatttggcctttctgaggcagggtttaatacagcatgtgttaccaagctgtttcctaccaggtcacacactgttgcagcacagggaggaatcaatgctgctctggggaacatggaggaggacaactggaggtggcatttctacgacaccgtgaagggctccgactggctgggggaccaggatgccatccactacatgacggagcaggcccccgccgccgtggtcgagctagaaaattatggcatgccgtttagcagaactgaagatgggaagatttatcagcgtgcatttggtggacagagcctcaagtttggaaagggcgggcaggcccatcggtgctgctgtgtggctgatcggactggccactcgctattgcacaccttatatggacggtctctgcgatatgataccagctattttgtggagtattttgccttggatctcctgatggagaacggggagtgccgtggtgtcatcgcactgtgcatagaggacgggtccatccatcgcataagagcaaagaacactgttgttgccacaggaggctacgggcgcacctacttcagctgcacgtctgcccacaccagcactggcgacggcacggccatgatcaccagggcaggccttccttgccaggacctagagtttgttcagttccaccctacaggcatatatggtgctggttgtctcattacggaaggatgtcgtggagagggaggcattctcattaacagtcaaggcgaaaggtttatggagcgatacgcccctgtcgcgaaggacctggcgtctagagatgtggtgtctcggtccatgactctggagatccgagaaggaagaggctgtggccctgagaaagatcacgtctacctgcagctgcaccacctacctccagagcagctggccacgcgcctgcctggcatttcagagacagccatgatcttcgctggcgtggacgtcacgaaggagccgatccctgtcctccccaccgtgcattataacatgggcggcattcccaccaactacaaggggcaggtcctgaggcacgtgaatggccaggatcagattgtgcccggcctgtacgcctgtggggaggccgcctgtgcctcggtacatggtgccaaccgcctcggggcaaactcgctcttggacctggttgtctttggtcgggcatgtgccctgagcatcgaagagtcatgcaggcctggagataaagtccctccaattaaaccaaacgctggggaagaatctgtcatgaatcttgacaaattgagatttgctgatggaagcataagaacatcggaactgcgactcagcatgcagaagtcaatgcaaaatcatgctgccgtgttccgtgtgggaagcgtgttgcaagaaggttgtgggaaaatcagcaagctctatggagacctaaagcacctgaagacgttcgaccggggaatggtctggaacacggacctggtggagaccctggagctgcagaacctgatgctgtgtgcgctgcagaccatctacggagcagaggcacggaaggagtcacggggcgcgcatgccagggaagactacaaggtgcggattgatgagtacgattactccaagcccatccaggggcaacagaagaagccctttgaggagcactggaggaagcacaccctgtcctatgtggacgttggcactgggaaggtcactctggaatatagacccgtgatcgacaaaactttgaacgaggctgactgtgccaccgtcccgccagccattcgctcctactgatgagacaagatgtggtgatgacagaatcagcttttgtaattatgtataatagctcatgcatgtgtccatgtcataactgtcttcatacgcttctgcactctggggaagaaggagtacattgaagggagattggcacctagtggctgggagcttgccaggaacccagtggccagggagcgtggcacttacctttgtcccttgcttcattcttgtgagatgataaaactgggcacagctcttaaataaaatataaatgaac

SEQ ID NO: 3 - protein, 280 aa, Homo sapiens
MAAVVALSLRRRLPATTLGGACLQASRGAQTAAATAPRIKKFAIYRWDPDKAGDKPHMQTYEVDLNKCGPMVLDALIKIKNEVDSTLTFRRSCREGICGSCAMNINGGNTLACTRRIDTNLNKVSKIYPLPHMYVIKDLVPDLSNFYAQYKSIEPYLKKKDESQEGKQQYLQSIEEREKLDGLYECILCACCSTSCPSYWWNGDKYLGPAVLMQAYRWMIDSRDDFTEERLAKLQDPFSLYRCHTIMNCTRTCPKGLNPGKAIAEIKKMMATYKEKKASV

SEQ ID NO: 4 - DNA, 1100 bp, Homo sapiens
ggcctcccacttggttgctcgtacgcggctagtgggtcctcagtggatgtaggctgggcgccgcgatgttcgacgggacaccggcggagagcgacctcggggttaaggggtggggctggacgtcaggagccaagatggcggcggtggtcgcactctccttgaggcgccggttgccggccacaacccttggcggagcctgcctgcaggcctcccgaggagcccagacagctgcagccacagctccccgtatcaagaaatttgccatctatcgatgggacccagacaaggctggagacaaacctcatatgcagacttataaggttgaccttaataaatgtggccccatggtattggatgctttaatcaagattaagaatgaagttgactctactttgaccttccgaagatcatgcagagaaggcatctgtggctcttgtgcaatgaacatcaatggaggcaacactctagcttgcacccgaaggattgacaccaacctcaataaggtctcaaaaatctaccctcttccacacatgtatgtgataaaggatcttgttcccgatttgagcaacttctatgcacagtacaaatccattgagccttatttgaagaagaaggatgaatctcaggaaggcaagcagcagtatctgcagtccatagaagagcgtgagaaactggacgggctctacgagtgcattctctgtgcctgctgtagcaccagctgccccagctactggtggaacggagacaaatatctggggcctgcagttcttatgcaggcctatcgctggatgattgactccagagatgacttcacagaggagcgcctggccaagctgcaggacccattctctctataccgctgccacaccatcatgaactgcacaaggacctgtcctaagggtctgaatccagggaaagctattgcagagatcaagaaaatgatggcaacctataaggagaagaaagcttcagtttaactgtttccatgctaaacatgatttataaccagctcagagctgaacataatttatatctaatttgagttcctttaaagatcttggttttccatgaatacagcatgtataataaaaattttaaga

SEQ ID NO: 5 - protein, 169 aa, Homo sapiens
MAALLLRHVGRHCLRAHFSPQLCIRNAVPLGTTAKEEMERFWNKNIGSNRPLSPHITIYSWSLPMAMSICHRGTGIALSAGVSLFGMSALLLPGNFESYLELVKSLCLGPALIHTAKFALVFPLMYHTWNGIRHLMWDLGKGLKIPQLYQSGVVVLVLTVLSSMGLAAM

SEQ ID NO: 6 - DNA, 1107 bp, Homo sapiens
  misc_feature (183)..(183): n is a, c, g, or t
agagaacattcctcctaccatcaccacacaatagaacctgattttctgtcattggaataagtggataataggttccatactgtgggttttgagaagggtaaaggtggggcataagggtagaagcgcttttctctagaatcatgctgagaggagatatctattcctttaggtagaattactttntgagacaggaactgttaatgtcctatttactgaaattccttttttttttttttgctttgtccacagatgtgggacctaggaaaaggcctgaagattccccagctataccagtctggagtggttgtcctggttcttactgtgttgtcctctatggggctggcagccatgtgaagaaaggaggctcccagcatcatcttcctacacattattacattcacccatctttctgtttgtcattcttatctccagcctgggaaaagttctccttatttgtttagatccttttgtattttcagatctccttggagcagtagagtacctggtagaccataatagtggaaaagggtctagttttccccttgtttctaaagatgaggtggctgcaaaaactccccttttttgcccacagcttgcctactctcggcctagaagcagttattctctctccatattgggctttgatttgtgctgagggtcagcttttggctccttcttcctgagacagtggaaacaatgccagctctgtggcttctgccctggggatgggccgggttggggggtgggttggtgaggctttgggtgccactgcctgtgggttgctggcttaaaggacaattctcttcattggtgagagcccaggccattaacacctacacagtgttattgaaagaagagaggtgggggtggaggggaattagtctgtcccagctagagggagataaagagggctagttagttcttggagcagctgcttttgaggagaaaatatatagctttggacacgaggaagatctagaaaattatcattgaacatattaatggttatttctttttcttggatttccagaaaagcctcttaattttatgctttctcatcgaagtaatgtaccctttttttctgaaactgaattaaatactcatttt

SEQ ID NO: 7 - protein, 159 aa, Homo sapiens
MAVLWRLSAVCGALGGRALLLRTPVVRPAHISAFLQDRPIPEWCGVQHIHLSPSHHSGSKAASLHWTSERVVSVLLLGLLPAAYLNPCSAMDYSLAAALTLHGHWGLGQVVTDYVHGDALQKAAKAGLLALSALTFAGLCYFNYHDVGICKAVAMLWKL

SEQ ID NO: 8 - DNA, 480 bp, Homo sapiens
atggcggttctctggaggctgagtgccgtttgcggtgccctaggaggccgagctctgttgcttcgaactccagtggtcagacctgctcatatctcagcatttcttcaggaccgacctatcccagaatggtgtggagtgcagcacatacacttgtcaccgagccaccattctggctccaaggctgcatctctccactggactagcgagagggttgtcagtgttttgctcctgggtctgcttccggctgcttatttgaatccttgctctgcgatggactattccctggctgcagccctcactcttcatggtcactggggccttggacaagttgttactgactatgttcatggggatgccttgcagaaagctgccaaggcagggcttttggcactttcagctttaacctttgctgggctttgctatttcaactatcacgatgtgggcatctgcaaagctgttgccatgctgtggaagctctag

SEQ ID NO: 9 - protein, 481 aa, Homo sapiens
MMLKSITESFATAIHGLKVGHLTDRVIQRSKRMILDTLGAGFLGTTTEVFHIASQYSKIYSSNISSTVWGQPDIRLPPTYAAFVNGVAIHSMDFDDTWHPATHPSGAVLPVLTALAEALPRSPKFSGLDLLLAFNVGIEVQGRLLHFAKEANDMPKRFHPPSVVGTLGSAAAASKFLGLSSTKCREALAIAVSHAGAPMANAATQTKPLHIGNAAKHGIEAAFLAMLGLQGNKQVLDLEAGFGAFYANYSPKVLPSIASYSWLLDQQDVAFKRFPAHLSTHWVADAAASVRKHLVAERALLPTDYIKRIVLRIPNVQYVNRPFPVSEHEARHSFQYVACAMLLDGGITVPSFHECQINRPQVRELLSKVELEYPPDNLPSFNILYCEISVTLKDGATFTDRSDTFYGHWRKPLSQEDLEEKFRANASKMLSWDTVESLIKIVKNLEDLEDCSVLTTLLKGPSPPEVASNSPACNNSITNLS

SEQ ID NO: 10 - DNA, 1443 bp, Homo sapiens
atgatgctcaagtctatcacagaaagctttgccacagcaatccatggcttgaaagtgggacacctgacagatcgtgttattcagaggagcaagaggatgattctagacactctgggtgctgggttcctgggaaccactacggaagtgtttcacatagccagccaatatagcaagatctacagttccaacatatccagcactgtttggggtcagccagacatcaggctcccgcccacatatgctgcttttgtgaacggtgtggctattcactccatggattttgatgacacgtggcaccctgccacccacccttctggggctgtccttcctgtcctcacagctttagcagaagccctgccaaggagtccaaagttttctggccttgacctgctgctggctttcaatgttggtattgaagtgcaaggccgattactgcatttcgccaaggaggccaatgacatgccaaagagattccatcccccttccgtggtaggaacgttgggtagtgctgctgctgcatccaagtttttaggacttagctcgacaaagtgccgagaagctctggccattgctgtttcccatgctggggcacccatggccaatgctgccacccagaccaagcccctccacattggcaatgctgccaagcatgggatagaagctgcatttttggcaatgttgggtctccaaggaaacaagcaggtcttggacttggaggcaggatttggggccttttatgccaactattccccaaaagtccttccaagcatagcttcctacagttggctgctggaccagcaggacgtggcctttaagcgttttcctgcacatttatctacccactgggtggcagacgcagctgcatctgtgagaaagcaccttgtagcagagagagccctgcttccaactgactacattaagagaattgtgctcaggataccaaatgtccagtatgtaaacaggccctttccagtttcggagcatgaagcccgtcattcattccagtatgtggcctgtgccatgctgcttgatggtggcatcactgtcccctcattccatgaatgccagatcaacaggccacaggtgagagagctgctcagtaaggtggagctggagtaccctccggacaacttgccaagcttcaacatactgtactgtgaaataagtgtcaccctcaaggatggagccaccttcacagatcgctctgataccttctatgggcactggagaaaaccactgagccaggaggacctagaggaaaagttcagagccaatgcctccaagatgctgtcctgggacacagtggaaagccttataaagatagtcaaaaatctagaagacctagaagactgttctgtgttaactacacttctcaaaggaccctctccaccagaggtagcttcaaactctccagcatgtaataattctatcacaaatctctcc